(19) United States
(12) Patent Application Publication
(10) Pub. No.: US 2025/0171729 A1
(43) Pub. Date: May 29, 2025

(54) BIOFERTILIZER COMPOSITIONS AND METHODS

(71) Applicant: KULA BIO, INC., Natick, MA (US)

(72) Inventors: Kelsey K. SAKIMOTO, Cambridge, MA (US); James P. KILGANNON, Somerville, MA (US); Elliot George ROSSOW, Somerville, MA (US); Meghan POWERS, Somerville, MA (US)

(21) Appl. No.: 18/839,882

(22) PCT Filed: Feb. 22, 2023
(86) PCT No.: PCT/US2023/063066

§ 371 Date: Aug. 20, 2024

Related U.S. Application Data
(60) Provisional application No. 63/313,188, filed on Feb. 23, 2022.

(51) Int. Cl. C12N 1/20; A01N 63/20; A01P 21/00

(52) U.S. Cl. CPC C12N 1/20; A01N 63/20; A01P 21/00; C12N 2500/16; C12N 2500/24; C12N 2500/30; C12N 2500/42

(57) ABSTRACT

The disclosure provides a growth medium and a culturing system for a nitrogen-fixing microorganism. The disclosure also provides methods of enhancing the accumulation of a microbial intracellular storage compound (MISC) in a nitrogen-fixing microorganism. The disclosure further provides biofertilizers comprising a nitrogen-fixing microorganism, as well as methods of improving and/or maintaining crop or plant yield, yield quality, or plant aesthetics, and/or improving soil health using the biofertilizers.

### FIELD OF THE DISCLOSURE

The disclosure relates to a growth medium and a culturing system for production of biofertilizers, and methods for preparing and using the biofertilizers to improve and/or maintain crop or plant yield, yield quality, or plant aesthetics, and/or improve soil health. The disclosure further relates to a growth medium composition and formulation, and biofertilizer composition and formulation.

### BACKGROUND OF THE DISCLOSURE

Microorganisms, including bacteria, fungi, protists and microalgae, are abundant in productive agricultural soils and common inoculants, mediating numerous plant- and soil-beneficial functions, such as nutrient cycling, pathogen inhibition, hormone signaling, environmental resilience, and bioremediation, among others. Like all biological entities, microorganisms require energy-dense biochemical substrates to drive their metabolic processes, including simple cell maintenance and viability, as well as the plant- and soil-beneficial functions. These substrates can take the form of carbon-based molecules derived from plant-based photosynthesis, in the form of sugars, organic acids or biopolymers. They can also come from the general pool of soil organic carbon (SOC, also called soil organic matter, SOM) that is replenished and depleted as a result of climate and agricultural practices. Plant residues and other organic matter, such as compost and manure, can increase the SOC pool, while intensive cultivation of soil, mono-cropping, and conventional farming practices that remove organic matter from agricultural soils will deplete the SOC.

The limited availability of metabolic substrates often bottlenecks plant- and soil-beneficial functions. Microorganisms exhibiting direct symbioses with plants, such as endophytes and root-nodule forming microorganisms, typically rely on the diversion of an already limited flux of plant-derived carbon compounds away from essential plant functions. The finite and highly competed-for pool of SOC presents similar challenges for both native and exogenously added soil microorganisms. In both metabolite limited scenarios, the limited flux of energy and nutrients reduces the rate, performance and efficacy of energy intensive biological processes, such as nitrogen fixation, phosphorus mobilization, carbon fixation, nutrient cycling, among others.

For agricultural biologics, often including live microorganisms, the availability of metabolites during processing, storage and transport can impede their efficacy even before soil/plant application. Beginning at the downstream processing, formulation, packaging, storage, transport and application of microorganisms from their ex situ production in a bioreactor or similar apparatus, the extended duration and non-optimal conditions present significant challenges to cell viability. Removed from the normal conditions for growth, cells begin to enter a death and dormancy phase having exhausted essential metabolites, decreasing the total number of viable effective cells. Measures to slow down microbial decline, such as chemical inhibitors, sporulation, or lowered temperatures may be effective, but present additional cost and complexity for biologics.

Supplemental metabolites can augment the limited plant and soil-derived sources, as well as provide an excess of nutrients during processing, storage and transport. Extracellular, exogenous metabolites, such as chemical and biologically-derived materials added to live cultures of microorganisms, can augment their viability and efficacy, but are non-specific to beneficial microbes and can be utilized by other contaminants, pathogens, and parasitic species. Intracellular, endogenous metabolites, such as polymeric storage compounds accumulated within a specific microorganism, can similarly fortify the viability and efficacy of agricultural biologics, but are consumed preferential by the specific microorganisms itself over competing microbes. Several microbial intracellular storage compounds (MISC), such as polyhydroxyalkanoates (PHAs), specifically polyhydroxybutyrate (PHB) and polyphosphates (PolyP), can be produced in both naturally-occurring and engineered microorganisms to fortify them with additional sources of carbon and metabolic energy. However, the accumulation of such MISC do not typically and reliably occur under existing conventional culturing systems.

The production of high levels of MISC in microorganisms of agricultural significance carries two requirements: 1) high cell density cultures and 2) simplified bioreactor/bioprocesses systems. High cell density cultures, here referring to microbial cultures with an optical density (OD600) value of about >10, are particularly desirable for agricultural applications where use of such concentrated suspensions of cells reduces prohibitive transport, application, and logistical burdens. Achieving high cell density cultures of microorganisms typically requires repeated additions of growth supportive nutrients in a continuous, fed batch, or semi-fed batch mode of operation. This series of one or more additions during culturing is required for nutrients in which a single or reduced number of additions to supply the total required nutrient loading would generate a concentration that exceeds growth supportive conditions. This is particularly true of nutrients whose maximum solubility or miscibility exceeds a level determined to be inhibitory of microbial growth (e.g., alcohols, anhydrous ammonia/ammonium hydroxide). Furthermore, the maximum cell densities of cultures are often limited practically by the accumulation of inhibitory metabolic byproducts, such as the counter-ions of certain nutrient sources (e.g., Ca²⁺ consumption from CaCl₂) leaves high concentrations of Cl⁻ as a byproduct) which creates osmotic stress, pH drift, or specific inhibitory pathways.

The practice of repeated additions of nutrients creates additional undesired bioprocess complexity. Typically, a series of controlled fluid handling pumps (e.g., peristaltic pumps) automate this process but at the same time increase the capital cost of the additional hardware, control system and software. These high capital expense bioreactors/bioprocess systems are incompatible with the requirements in agriculture for low-cost, simple ease-of-use bioprocesses. Additionally, the need to work with highly concentrated sources of key nutrients (such as ammonium hydroxide as a nitrogen source) presents additional material compatibility challenges and chemical safety hazards.

Use of biofertilizers as an alternative to synthetic fertilizer application provides a promising solution to the negative environmental impacts of excess nitrogen losses from agricultural systems. Nitrogen (N) is often the most limiting terrestrial nutrient, particularly in agricultural systems, and as such is often applied to crops as synthetic fertilizer to boost productivity. However, due to a variety of factors including plant demand vs. application timing, cropping systems often only use ˜40% of the applied N with the excess N lost through leaching and denitrification. These N losses not only result in negative environmental consequences, including toxic algal blooms and production of nitrous oxide, a potent greenhouse gas, but also greatly reduce the cost effectiveness of cropping systems. Biofertilizers represent a cost-effective and sustainable alternative to synthetic N fertilizer application. Relying on microorganisms, often with biological nitrogen fixation (BNF) potential, as a N source, biofertilizers can achieve a slower release of N that is often better timed with plant demand.

To overcome the challenges described above, biofertilizers with high cell density cultures and enhanced accumulation of MISC are needed to enrich soils and/or soil microbiomes, and to enhance crop or plant yields, yield quality, aesthetics, and other characteristics.

### BRIEF SUMMARY OF THE DISCLOSURE

The disclosure relates to a growth medium for a nitrogen-fixing microorganism comprising a compound nutrient source, a salt, a buffer, a carbon source, and a metal source. In one aspect, the compound nutrient source comprises two or more constituent components. In another aspect, the salt comprises a soluble salt of the constituent component. In another aspect, the compound nutrient source has a solubility lower than the solubility of the soluble salt of the constituent component. In another aspect, the concentration of the constituent component salt is below the toxicity level of the nitrogen-fixing microorganism. In some aspects, the compound nutrient source comprises ammonium (NH₄⁺), magnesium (Mg²⁺), and phosphate (H₂PO₄⁻, HPO₄²⁻, PO₄³⁻). In some aspects, the compound nutrient source is selected from ammonium magnesium phosphate (MgNH₄PO₄) or a hydrate thereof. In some aspects, the compound nutrient source is ammonium magnesium phosphate hexahydrate (MgNH₄PO₄·6H₂O).

In some aspects, the compound nutrient source further comprises metal carbonate. In some aspects, the metal carbonate comprises a divalent cationic metal. In some aspects, the divalent cationic metal comprises Ca²⁺, Mg²⁺, Fe²⁺, Ni²⁺, or Co²⁺, or a combination thereof. In one aspect, the divalent cation metal is Fe²⁺. In another aspect, the medium comprises ferric citrate and ammonium bicarbonate ((NH₄)HCO₃).

In some aspects, the constituent component salt is selected from a soluble salt of ammonium (NH₄⁺), magnesium (Mg²⁺), or phosphate (H₂PO₄⁻, HPO₄²⁻, PO₄³⁻), or a combination thereof. In some aspects, the medium comprises one of each of the soluble salts of ammonium (NH₄⁺), magnesium (Mg²⁺), and phosphate (H₂PO₄, HPO₄²⁻, PO₄³⁻). In some aspects, the medium comprises potassium phosphate monobasic (K₂HPO₄), potassium phosphate dibasic (KH₂PO₄), magnesium sulfate heptahydrate (MgSO₄·7H₂O), or ammonium sulfate ((NH₄)₂SO₄), or a combination thereof.

In some aspects, the concentration of ammonium is less than about 20 mM. In one aspect, the concentration of ammonium is from about 5 mM to about 12 mM. In one aspect, the concentration of ammonium is about 5 mM. In one aspect, the concentration of ammonium is about 6 mM. In one aspect, the concentration of ammonium is about 7 mM. In one aspect, the concentration of ammonium is about 8 mM. In one aspect, the concentration of ammonium is about 9 mM. In one aspect, the concentration of ammonium is about 10 mM. In one aspect, the concentration of ammonium is about 11 mM. In one aspect, the concentration of ammonium is about 12 mM.

In some aspects, the medium further comprises a soluble salt of calcium (Ca²⁺). In one aspect, the salt is CaSO₄.

In some aspects, the medium comprises a carbon source. In one aspect, the carbon source is selected from an autotrophic carbon source, or a heterotrophic carbon source, or a combination thereof. In one aspect, the carbon source is an autotrophic carbon source. In one aspect, the autotrophic carbon source is methanol. In another aspect, the carbon source is a heterotrophic carbon source. In some aspects, the heterotrophic carbon source is selected from glucose, succinate, butyrate, pyruvate, propanol, acetate, fructose, sucrose, butanol, propanol, ethanol, pentanol, rich broth, L-amino acids, arabinose, sucrose, dextrin, dextrose, lactose, maltose, fucose, galactose, mannose, saccharose, or xylose, or a combination thereof.

In some aspects, the medium comprises a metal source. In some aspects, the metal source comprises a trace metal or semi-metal source. In one aspect, the trace metal or semi-metal source is ferric citrate, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), or nickel (II) sulfate hexahydrate (NiSO₄·6H₂O), or a combination thereof. In one aspect, the trace metal or semi-metal source is a combination of boric acid, manganese (II) sulfate monohydrate, sodium molybdate, zinc sulfate, copper (II) nitrate pentahydrate, and nickel(II) sulfate.

In some aspects, the medium further comprises a nitrogen-fixing microorganism. In some aspects, the nitrogen-fixing microorganism expresses nitrogenase. In some aspect, the nitrogen-fixing microorganism accumulates a microbial intracellular storage compound (MISC). In some aspects, the nitrogen-fixing microorganism expresses nitrogenase and accumulates a MISC. In some aspects, the MISC comprises a polyhydroxyalkanoate (PHA), a polyphosphate (PolyP), or a lipid, or a combination thereof. In some aspects, the MISC is a PHA. In some aspects, the PHA is polyhydroxybutyrate (PHB), poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

In some aspects, the nitrogen-fixing microorganism comprises bacteria. In some aspects, the nitrogen-fixing microorganism is a PHB-producing bacteria. In other aspects, the nitrogen-fixing microorganism comprises archaea. In other aspects, the nitrogen-fixing microorganism comprises fungi.

In some aspects, the nitrogen-fixing microorganism comprises one or more strains of Acidiphilium species, Acidiphilium multivorum, Alcaligenes species, Alcaligenes paradoxus, Arthrobacter species, Azohydromonas species, Azohydromonas australica, Azohydromonas species, Azohydromonas lata, Azospirillum species, Azospirillum amazonsense, Azospirillum lipoferum, Azospirillum thiophilum, Azotobacter vinelandii, Beggiatoa species, Beggiatoa alba, Beijerinckia species, Beijerinckia mobilis, Bradyrhizobium species, Bradyrhizobium elnakii, Bradyrhizobium japonicum, Bradyrhizobium japonicum (strain USDA 122), Burkholderia species, Burkholderia vietnameiensis, Cupriavidus species, Cupriavidus necator, Derxia species, Derxia gummosa, Herbaspirillum species, Herbaspirillum autrotrophicum, Hydrogenophaga species, Hydrogenophaga pseudoflava, Mesorhizobium species, Mesorhizobium alhagi, Methylibium species, Methylibium petroleiphilum, Methylocapsa species, Methylocapsa aurea, Methyloferula species, Methyloferula stellate, Methyloversatilis species, Methyloversatilis universalis, Microcyclus species, Microcyclus aquaticus, Microcyclus ebruneus, Nitrosococcus species, Nitrosococcus oceani, Nitrosomonas communis, Nitrospirillum amazonense, Nocardia species, Nocardia autotrophica, Nocardia opaca, Oligotropha species, Oligotropha carboxidovorans, Pannonibacter species, Pannonibacter phragmitetus, Paracoccus species, Paracoccus denitrificans, Paracoccus pantrophus, Paracoccus yeei, Pelagibaca species, Pelagibaca bermudensis, Pseudomonas species, Pseudomonas facilis, Pseudooceanicola species, Pseudooceanicola atlanticus, Ralstonia species, Ralstonia eutropha, Renobacter species, Renobacter vacuolatum, Rhizobium species, Rhizobium gallicum, Rhizobium japonicum, Rhodobacter species, Rhodobacter capsulatus, Rhodobacter sphaeroides, Rhodomicrobium species, Rhodomicrobium vannielii, Rubrivivax species, Rubrivivax gelatinosus, Salipiger species, Salipiger mucosus, Sinorhizobium species, Sinorhizobium americanum, Sinorhizobium fredii, Sinorhizobium meliloti, Skermanella species, Skermanella stibiiresistens, Stappia species, Stappia aggregate, Thauera species, Thauera humireducens, Variovorax species, Variovorax paradoxus, Xanthobacter species, or Xanthobacter autotrophicus. In one aspect, the nitrogen-fixing microorganism is Xanthobacter autotrophicus. In another aspect, the nitrogen-fixing microorganism is Ralstonia eutropha.

The disclosure relates to a medium comprising ammonium magnesium phosphate hexahydrate (MgNH₄PO₄·6H₂O), ferric citrate, ammonium bicarbonate ((NH₄)HCO₃), potassium phosphate monobasic (K₂HPO₄), potassium phosphate dibasic (KH₂PO₄), magnesium sulfate heptahydrate (MgSO₄·7H₂O), calcium sulfate dihydrate (CaSO₄·2H₂O), ammonium sulfate ((NH₄)₂SO₄), methanol, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O).

The disclosure further relates to a medium comprising ammonium dihydrogen phosphate ((NH₄)H₂PO₄), magnesium hydroxide (Mg(OH)₂), ferric citrate, ammonium bicarbonate ((NH₄)HCO₃), potassium phosphate monobasic (K₂HPO₄), potassium phosphate dibasic (KH₂PO₄), magnesium sulfate heptahydrate (MgSO₄·7H₂O), calcium sulfate dihydrate (CaSO₄·2H₂O), ammonium sulfate ((NH₄)₂SO₄), methanol, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O). The disclosure further relates to a biofertilizer comprising the medium discussed herein.

The disclosure also relates to methods of preparing a nitrogen-fixing microorganism with additional and readily-available sources of carbon and energy to augment the viability and vitality of the microorganism. The disclosed methods of enhancing the accumulation of a MISC in a nitrogen-fixing microorganism comprise a) growing the nitrogen-fixing microorganism in a growth medium comprising a compound nutrient source, a salt, a buffer, a carbon source, and a metal source, and b) adding a carbon source in an amount that exceeds the amount sufficient for the growth of the microorganism, thereby enhancing the accumulation of the MISC in the microorganism. In one aspect, the compound nutrient source comprises three or more constituent components. In one aspect, the salt comprises a soluble salt of the constituent component. In one aspect, the compound nutrient source has a solubility lower than the solubility of the soluble salt of the constituent component. In one aspect, the concentration of the constituent component salt is below the toxicity level of the nitrogen-fixing microorganism. In one aspect, the culture of the nitrogen-fixing microorganism is grown to a specified concentration measured by an optical density at 600 nm (OD₆₀₀). In another aspect, the compound nutrient source is added in a single batch.

In some aspects, the method further comprises inoculating the medium with an initial culture of the nitrogen-fixing microorganism. In some aspects, the medium is inoculated with the initial culture of the nitrogen fixing microorganism. In some aspects, the medium is inoculated with the initial culture of the nitrogen fixing microorganism having an OD₆₀₀ greater than about 2. In other aspects, the culture of the microorganism has an OD₆₀₀ greater than about 0, greater than about 2, greater than about 5, greater than about 7, greater than about 10, greater than about 15, greater than about 20, greater than about 25, greater than about 30, greater than about 35, or greater than about 40. In other aspects, the culture of the microorganism has an OD₆₀₀ of a range from about 0 to about 40. In other aspects, the culture of the microorganism has an OD₆₀₀ from about 0 to about 1, from about 0 to about 2, from about 0 to about 3, from about 0 to about 4, from about 0 to about 5, from about 2 to about 3, from about 2 to about 4, from about 2 to about 5, from about 3 to about 4, from about 3 to about 5, from about 3 to about 7, from about 4 to about 7, from about 4 to about 8, from about 4 to about 10, from about 5 to about 10, from about 5 to about 12, from about 5 to about 15, from about 5 to about 20, from about 7 to about 10, from about 7 to about 12, from about 7 to about 15, from about 7 to about 20, from about 8 to about 10, from about 8 to about 12, from about 8 to about 15, from about 8 to about 20, from about 9 to about 10, from about 9 to about 12, from about 9 to about 15, from about 9 to about 20, from about 10 to about 12, from about 10 to about 15, from about 10 to about 20, from about 12 to about 15, from about 12 to about 20, from about 12 to about 23, from about 12 to about 25, from about 15 to about 20, from about 15 to about 23, from about 15 to about 25, from about 17 to about 20, from about 17 to about 23, from about 17 to about 25, from about 17 to about 27, from about 20 to about 23, from about 20 to about 25, from about 20 to about 27, from about 20 to about 30, from about 23 to about 25, from about 23 to about 27, from about 23 to about 30, from about 23 to about 33, from about 25 to about 27, from about 25 to about 30, from about 25 to about 33, from about 25 to about 35, from about 27 to about 30, from about 27 to about 33, from about 27 to about 35, from about 27 to about 37, from about 30 to about 33, from about 30 to about 35, from about 30 to about 37, from about 30 to about 40, from about 33 to about 35, from about 33 to about 37, from about 33 to about 40, from about 33 to about 43, from about 35 to about 37, from about 35 to about 40, from about 35 to about 43, from about 35 to about 45, from about 37 to about 40, from about 37 to about 43, from about 37 to about 45, from about 40 to about 43, from about 40 to about 45, or from about 43 to about 45. In other aspects, the culture of the microorganism has an OD₆₀₀ of about 0, about 2, about 3, about 4, about 5, about 7, about 8, about 9, about 10, about 12, about 15, about 20, about 23, about 25, about 27, about 30, about 33, about 35, about 37, about 40, about 43, or about 45. In one aspect, the culture of the microorganism has an OD₆₀₀ greater than about 2. In one aspect, the culture of the microorganism has an OD₆₀₀ from about 2 to about 4. In another aspect, the culture of the microorganism has an OD₆₀₀ of about 2. In another aspect, the culture of the microorganism has an OD₆₀₀ greater than about 20. In another aspect, the culture of the microorganism has an OD₆₀₀ of about 20. In one aspect, the culture of the microorganism has an OD₆₀₀ greater than about 25. In another aspect, the culture of the microorganism has an OD₆₀₀ of about 25.

In some aspects, the MISC accumulated in the microorganism is a PHA, a PolyP, or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB. In some aspects, the accumulation of MISC, such as PHB, is greater than about 10%, greater than about 12%, greater than about 15%, greater than about 20%, greater than about 25%, greater than about 30%, greater than about 35%, or greater than about 40%.

In some aspects, the accumulation of MISC, such as PHB, is greater than about 25% to about 40%. In other aspects, the accumulation of MISC is greater than about 25%.

In some aspects, the accumulation of MISC, such as PHB, is from about 10% to about 12%, from about 10% to about 15%, from about 10% to about 20%, from about 10% to about 25%, from about 12% to about 15%, from about 12% to about 20%, from about 12% to about 25%, from about 12% to about 30%, from about 15% to about 20%, from about 15% to about 25%, from about 15% to about 30%, from about 15% to about 35%, from about 20% to about 25%, from about 20% to about 30%, from about 20% to about 35%, from about 20% to about 40%, from about 25% to about 30%, from about 25% to about 35%, from about 25% to about 40%, from about 30% to about 35%, from about 30% to about 40%, or from about 35% to about 40%.

In other aspects, the accumulation of MISC, such as PHB, is about 10%, about 12%, about 15%, about 20%, about 25%, about 30%, about 35%, or about 40%.

In one aspect, the accumulation of MISC, such as PHB, is about 25%. In one aspect, the accumulation of MISC is about 30%. In one aspect, the accumulation of MISC is about 40%.

In some aspects, the accumulation of MISC therein can be measured as the OD₆₀₀ of the culture of the microorganism after bleach digest of microorganism over the original OD₆₀₀ of the culture of the microorganism.

The disclosure also relates to a biofertilizer for use to improve and/or maintain crop or plant yield, yield quality, or plant aesthetics, and/or improve soil health comprising a nitrogen-fixing microorganism. In one aspect, the microorganism has an accumulation of a MISC, such as PHB, greater than about 10%.

In some aspects, the biofertilizer is a liquid, a solid, or a semisolid, or a combination thereof.

In some aspects, the MISC is a PHA, a PolyP, or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof.

In some aspects, the PHA is PHB. In some aspects, the accumulation of MISC, such as PHB, is greater than about 10%, greater than about 12%, greater than about 15%, greater than about 20%, greater than about 25%, greater than about 30%, greater than about 35%, or greater than about 40%. In one aspect, the accumulation of MISC is greater than about 25%. In another aspect, the accumulation of MISC is greater than about 30%. In another aspect, the accumulation of MISC is greater than about 40%.

In some aspects, the accumulation of MISC is from about 10% to about 12%, from about 10% to about 15%, from about 10% to about 20%, from about 10% to about 25%, from about 12% to about 15%, from about 12% to about 20%, from about 12% to about 25%, from about 12% to about 30%, from about 15% to about 20%, from about 15% to about 25%, from about 15% to about 30%, from about 15% to about 35%, from about 20% to about 25%, from about 20% to about 30%, from about 20% to about 35%, from about 20% to about 40%, from about 25% to about 30%, from about 25% to about 35%, from about 25% to about 40%, from about 30% to about 35%, from about 30% to about 40%, from about 30% to about 45%, from about 30% to about 50%, or from about 35% to about 40%.

In some aspects, the accumulation of MISC is about 10%, about 12%, about 15%, about 20%, about 25%, about 30%, about 35%, or about 40%.

In one aspect, the accumulation of MISC is about 25%. In one aspect, the accumulation of MISC is about 30%. In one aspect, the accumulation of MISC is about 40%.

In some aspects, the accumulation of MISC discussed herein can be measured as the OD₆₀₀ of the culture of the microorganism after bleach digest of the microorganism over the OD₆₀₀ of the culture of the microorganism before bleach digest.

In some aspects, the nitrogen-fixing microorganism in the biofertilizer comprises bacteria. In some aspects, the nitrogen-fixing microorganism is a PHB-producing bacteria. In other aspects, the nitrogen-fixing microorganism comprises archaea. In other aspects, the nitrogen-fixing microorganism comprises fungi.

In some aspects, the nitrogen-fixing microorganism in the biofertilizer comprises one or more strains of Acidiphilium species, Acidiphilium multivorum, Alcaligenes species, Alcaligenes paradoxus, Arthrobacter species, Azohydromonas species, Azohydromonas australica, Azohydromonas species, Azohydromonas lata, Azospirillum species, Azospirillum amazonsense, Azospirillum lipoferum, Azospirillum thiophilum, Azotobacter vinelandii, Beggiatoa species, Beggiatoa alba, Beijerinckia species, Beijerinckia mobilis, Bradyrhizobium species, Bradyrhizobium elnakii, Bradyrhizobium japonicum, Bradyrhizobium japonicum (strain USDA 122), Burkholderia species, Burkholderia vietnameiensis, Cupriavidus species, Cupriavidus necator, Derxia species, Derxia gummosa, Herbaspirillum species, Herbaspirillum autrotrophicum, Hydrogenophaga species, Hydrogenophaga pseudoflava, Mesorhizobium species, Mesorhizobium alhagi, Methylibium species, Methylibium petroleiphilum, Methylocapsa species, Methylocapsa aurea, Methyloferula species, Methyloferula stellate, Methyloversatilis species, Methyloversatilis universalis, Microcyclus species, Microcyclus aquaticus, Microcyclus ebruneus, Nitrosococcus species, Nitrosococcus oceani, Nitrosomonas communis, Nitrospirillum amazonense, Nocardia species, Nocardia autotrophica, Nocardia opaca, Oligotropha species, Oligotropha carboxidovorans, Pannonibacter species, Pannonibacter phragmitetus, Paracoccus species, Paracoccus denitrificans, Paracoccus pantrophus, Paracoccus yeei, Pelagibaca species, Pelagibaca bermudensis, Pseudomonas species, Pseudomonas facilis, Pseudooceanicola species, Pseudooceanicola atlanticus, Ralstonia species, Ralstonia eutropha, Renobacter species, Renobacter vacuolatum, Rhizobium species, Rhizobium gallicum, Rhizobium japonicum, Rhodobacter species, Rhodobacter capsulatus, Rhodobacter sphaeroides, Rhodomicrobium species, Rhodomicrobium vannielii, Rubrivivax species, Rubrivivax gelatinosus, Salipiger species, Salipiger mucosus, Sinorhizobium species, Sinorhizobium americanum, Sinorhizobium fredii, Sinorhizobium meliloti, Skermanella species, Skermanella stibiiresistens, Stappia species, Stappia aggregate, Thauera species, Thauera humireducens, Variovorax species, Variovorax paradoxus, Xanthobacter species, and Xanthobacter autotrophicus, and any combinations thereof.

In one aspect, the nitrogen-fixing microorganism in the biofertilizer is Xanthobacter autotrophicus. In another aspect, the nitrogen-fixing microorganism is Ralstonia eutropha. In some aspects, the nitrogen-fixing microorganism in the biofertilizer is Azotobacter vinelandii.

The biofertilizers discussed herein can be applied to a broad portfolio of plants and crops. In some aspects, the crop or plant comprises one or more crops or plants from the following families: Asteraceae, Poaceae, Brassicaceae, Cucurbitaceae, Solanaceae, Rosaceae, Cannabaceae, Poaceae, Amaranthaceae, Amaryllidaceae, Polygonaceae, Liliaceae, Lamiaceae, Ericaceae or Fabaceae. In some aspects, the crop or plant comprises one or more of the following crops or plants: a lettuce (Family: Asteraceae), a corn (Family: Poaceae), a cabbage (Family: Brassicaceae), a kale (Family: Brassicaceae), a cucumber (Family: Cucurbitaceae), a pepper (Family: Solanaceae), a tomato (Family: Solanaceae), a berry (Family: Rosaceae), a hemp (Family: Cannabaceae), a grass (Family: Poaceae), a turf, a beet (Family: Amaranthaceae), a squash (Family: Cucurbitaceae), an onion (Family: Amaryllidaceae), or a soybean (Family: Fabaceae). In some aspects, the crop or plant comprises one or more of the following crops or plants: a baby leaf lettuce, a head lettuce, a sweet corn, a sweet pepper, a strawberry, or a raspberry. In one aspect, the crop or plant comprises a baby leaf lettuce. In one aspect, the crop or plant comprises a head lettuce. In one aspect, the crop or plant comprises a sweet corn. In another aspect, the crop or plant comprises a sweet pepper. In another aspect, the crop or plant comprises a strawberry. In another aspect, the crop or plant comprises a raspberry.

In some aspects, the biofertilizer is stable for a period ranging at least from about 1 to about 2 months, from about 2 months to about 6 months, from about 6 months to about 12 months, or about 12 months or more at room temperature or at about 4° C. In some aspects, the biofertilizer is stable for at least about 1 month, at least about 2 months, at least about 6 months, or at least about 12 months at room temperature or at about 4° C. In other aspects, the OD₆₀₀ of the biofertilizer changes within ±10% during a period of at least about 1 month, at least about 2 months, at least about 6 months, or at least about 12 months at room temperature or at about 4° C. In other aspects, the OD₆₀₀ of the biofertilizer changes within ±10% during a period ranging at least from about 1 to about 2 months, from about 2 months to about 6 months, from about 6 months to about 12 months, or about 12 months or more at room temperature or at about 4° C. In other aspects, the amount of MISC changes within ±10% during a period of at least about 1 month, at least about 2 months, at least about 6 months, or at least about 12 months at room temperature or at about 4° C. In other aspects, the amount of MISC changes within ±10% during a period ranging at least from about 1 to about 2 months, from about 2 months to about 6 months, from about 6 months to about 12 months, or about 12 months or more at room temperature or at about 4° C.

The disclosure further relates to a method of improving and/or maintaining crop or plant yield, yield quality, or plant aesthetics and/or improving soil health comprising administered the biofertilizer discussed herein to the crop or plant.

In some aspects, the crop or plant comprises one or more crops or plants from the following families: Asteraceae, Poaceae, Brassicaceae, Cucurbitaceae, Solanaceae, Rosaceae, Cannabaceae, Poaceae, Amaranthaceae, Amaryllidaceae, Polygonaceae, Liliaceae, Lamiaceae, Ericaceae or Fabaceae.

In other aspects, the crop or plant comprises one or more of the following crops or plants: a lettuce (Family: Asteraceae), a corn (Family: Poaceae), a cabbage (Family: Brassicaceae), a kale (Family: Brassicaceae), a cucumber (Family: Cucurbitaceae), a pepper (Family: Solanaceae), a tomato (Family: Solanaceae), a berry (Family: Rosaceae), a hemp (Family: Cannabaceae), a grass (Family: Poaceae), a turf, a beet (Family: Amaranthaceae), a squash (Family: Cucurbitaceae), an onion (Family: Amaryllidaceae), or a soybean (Family: Fabaceae).

In other aspects, the crop or plant comprises one or more of the following crops or plants: a baby leaf lettuce, a head lettuce, a sweet corn, a sweet pepper, a strawberry, or a raspberry.

In some aspects, disclosed herein is a method of improving soil health comprising applying any of the biofertilizers discussed herein to a crop or plant.

In some aspects, soil health is improved by reducing nitrogen leaching in the soil. In some aspects, the nitrogen leaching in the soil is reduced relative to soil that has not been administered the biofertilizer.

In some aspects, soil health is improved by increasing biological nitrogen fixation in the soil. In some aspects, the biological nitrogen fixation is increased relative to soil that has not been administered the biofertilizer.

### BRIEF DESCRIPTION OF THE DRAWINGS/FIGURES

FIG. 1A provides a schematic diagram of the mechanisms and processes improving plant and soil health by the biofertilizer of the present disclosure. FIG. 1B provides a schematic diagram of the experimental comparison of the biofertilizer microorganisms with an enhanced accumulation of the PHB and the ones without. The % PHB is measured as OD₆₀₀ after the microorganism cells are digested with bleach.

FIG. 2 relates to the preparation of biofertilizer products. FIG. 2 plots the OD₆₀₀ (square) and the PHB % (circle) as a function of days post inoculation (the duration of the biofertilizer preparation).

FIGS. 3A and 3B provide a comparison of stability of biofertilizer using colony forming units (CFU) viability assay.

FIGS. 4A and 4B provide an example of dosing calibration of the biofertilizer (BF). FIG. 4A shows the fresh weights of the second harvest from baby leaf lettuce plants with increasing dosages of biofertilizer. A linear growth response with increasing dosages of biofertilizers in the fresh weights was observed. At 16 times dosing, the biofertilizer outperformed conventional fertilizer. Calcium nitrate or urea were applied to conventional (positive control) plants at the rate of about 20 lb nitrogen/acre. FIG. 4B shows the carry-over fertilization compared to the first harvests of no fertilizer application. At 8 times dosing or higher, the carry-over fertilization outperformed calcium nitrate fertilization and at 2 times dosing or higher, the carry-over fertilization resulted in larger fresher weights than urea fertilization.

FIGS. 5A, 5B, 5C and 5D provide results of a field trial using the biofertilizer (BF) of the present disclosure on romaine lettuce plants. FIG. 5A shows the lettuce fresh weights (kg) harvested from romaine lettuce plants with different ratios of biofertilizer:urea ammonium nitrate (UAN) applications. FIG. 5B shows the lettuce pre-sidedress nitrate test (PSNT) values of the lettuce plants with different ratios of biofertilizer:UAN applications. The low PSNT values of biofertilizer applications indicate low runoff potential and the low risk of over-fertilization using biofertilizers. FIG. 5C shows the biomass Total Kjeldahl nitrogen (TKN) of the lettuce plants with different ratios of biofertilizer:UAN applications. FIG. 5D depicts the pictures of the 50:50 and 0:100 plots of romaine lettuce plants, both having the fertilization rates of 100 lb N/acre.

FIGS. 6A, 6B and 6C provide an example of a field trial applying the biofertilizer (BF) on sweet corn plants. FIG. 6A shows the corn ear fresh weights (kg) harvested from sweet corn plants with different ratios of biofertilizer:UAN applications. FIG. 6B shows the corn PSNT values of the sweet corn plants with different ratios of biofertilizer:UAN applications. FIG. 6C shows the corn-stalk nitrate test (CSNT) of the corn plants with different ratios of biofertilizer:UAN applications.

FIGS. 7A, 7B, 7C, 7D and 7E provide biofertilizer (BF) performance testing results across wide variety of vegetable and fruit crops and plants. The fertilizers were applied every 2 weeks. The nitrate stimulation trends were observed across a wide portfolio of crops. FIG. 7A shows the cabbage fresh weights (g) across 4 applications: no fertilizer, calcium nitrate, biofertilizer, and co-application of biofertilizer and calcium nitrate. The total fertilization rate was at 180 lb nitrogen/acre for all applications. The dark data dots indicate the total weight of the cabbage plants while the light dots indicate the cabbage head weights. FIG. 7B shows the cucumber cumulative fruit weights (g) across applications of biofertilizer only, 50:50 biofertilizer:calcium nitrate, and calcium nitrate only. The total fertilization rate was at 116 lb nitrogen/acre for all applications. FIG. 7C shows the harvested pepper yields (g) from sweet pepper plants across applications of 50:50 biofertilizer:calcium nitrate, calcium nitrate only, biofertilizer only, and no fertilizer. The total fertilization rate was at 275 lb nitrogen/acre for all applications. There were three replicates for each application. The lower capital letters at the right end of the data lines indicate significant differences between fertilization using Tukey's HSD test (p<0.05). FIG. 7D shows the total harvested fruit weights (g) from cherry tomato plants across applications of 50:50 biofertilizer:calcium nitrate, biofertilizer only (lab grown), calcium nitrate only, biofertilizer only (reactor grown), and no fertilizer. The lab-grown biofertilizer were prepared with residual nitrate. The total fertilization rate was at 120 lb nitrogen/acre for all applications. FIG. 7E shows the total fruit weight produced (g) from strawberry plants across applications of biofertilizer only, no fertilizer, and calcium nitrate only. The total fertilization rate was at 200 lb nitrogen/acre for all applications. Specifically, the fertilization rate was 18 lb nitrogen per every 2 weeks.

FIGS. 8A, 8B, 8C, 8D, 8E and 8F provide an example of a greenhouse trial of biofertilizer (BF) applications on tomato plants. The fertilizer applications were conducted every 2 weeks. The 100% GS was 150 lb nitrogen/acre. FIG. 8A shows the cumulative yields (g) from tomato plants applied with different ratios of biofertilizer:UAN. The ratios of biofertilizer:UAN (% GS) were 0:0, 0:50, 0:75, 0:100, 25:75, 50:50, 75:25 and 100:25. Each ratio had 8 replicates. The lowercase letters above the data bars indicate significant differences between fertilization using Tukey's HSD test (p<0.05). FIG. 8B shows the comparison of different blend dosages. FIG. 8C shows the comparison of vegetative biomass and fruit biomass from tomato plants applied with different ratios of biofertilizer:UAN. The ratios of biofertilizer:UAN (% GS) were 0:0, 0:50, 0:75, 0:100, 25:75, 50:50, 75:25 and 100:25. FIG. 8D shows the cumulative yields (g) from tomato plants applied with different ratios of biofertilizer:UAN. The ratios of biofertilizer:UAN (% GS) were 0:100, 25:75, 50:50, 75:25, and 100:25. The typical yield is defined as the final yield produced by grower standard practices (0:100). FIGS. 8E and 8F depict the whole plants and the fruit, respectively, across fertilizer applications with different ratios of biofertilizer (BF in photos):UAN at 75:25, 50:50, 25:75, and 0:100.

FIG. 9 shows a greenhouse trial of biofertilizer (BF) applications on head lettuce plants to optimize the formulations, timing, and application methods. Biofertilizers (enhanced with 15-20% intracellular PHB) and calcium nitrate controls were applied sequentially at the time of transplantation (at the rate of 60 lb nitrogen/acre) and by sidedressing (at the rate of 40 lb nitrogen/acre), or both, as indicated in FIG. 9.

FIGS. 10A, 10B and 10C show a greenhouse trial with grass-type crop, orchardgrass, to test biofertilizer (BF) efficiency at different doses from about 25 to about 150 lb nitrogen/acre. Calcium nitrate was used as control. The lower capital letters above the data bars indicate significant differences between fertilization using Tukey's HSD test (p<0.05). FIG. 10A shows the fresh yields (g/pot) across different fertilizer applications from the second harvest. FIG. 10B shows the fresh yields (g/pot) across different fertilizer applications from the third harvest. FIG. 10C shows the combined results of fresh yields (g/pot) across different fertilizer applications from the second and third harvests.

FIGS. 11A and 11B show a representative example with baby leaf lettuce to compare the effects of soil quality and PHB content on biofertilizer (BF) efficacy. There were two harvests every 2 weeks for each plant. The fertilization applications include no fertilizer, calcium nitrate only, biofertilizer enhanced with 3 wt. % intracellular PHB only, biofertilizer enhanced with 15 wt. % intracellular PHB only, 50:50 3 wt. % biofertilizer:calcium citrate, and 50:50 15 wt. % biofertilizer:calcium citrate. FIG. 11A shows the lettuce yields (g) from the baby leaf lettuce grown in the soil not amended with different fertilization applications. FIG. 11B shows the lettuce yields (g) from the baby leaf lettuce grown in the compost amended soil with different fertilization applications.

FIGS. 12A and 12B show a representative example with beefsteak tomato to compare the effects of biofertilizer (BF) and synthetic fertilizer blend on fertilizer efficacy. Yield and quality (brix, pH, color, size) of tomato fruits were assessed over three harvest periods, as demonstrated in FIG. 12A. The fertilizer applications include 100% synthetic fertilizer following Grower Standard Practice (GSP) recommended Total Nitrogen (TN) rate of 100 lbs N/acre, 50% synthetic fertilizer under GSP, 20% synthetic fertilizer under GSP, 20% biofertilizer: 80% synthetic fertilizer, 50% biofertilizer: 50% synthetic fertilizer, and 80% biofertilizer: 20% synthetic fertilizer. Data shown are averages for each applications from six replicate plots. The synthetic fertilizer are made of Urea-Ammonium-Nitrate (UAN) and Calcium Ammonium-Nitrate (CAN). The biofertilizer was enhanced with intracellular PHB ranging from 5.5% to 21.7% and had OD600 ranging from 18.5 to 28.5, varying among preparation batches. FIG. 12B shows the tomato plants with 50% synthetic fertilizer application (top) and a 50:50 blend of biofertilizer and synthetic fertilizer.

FIGS. 13A and 13B show leaching trials comparing cumulative nitrate leached over time from soil columns treated with synthetic fertilizers, biofertilizer (BF) of the present disclosure, or blends of both. The applications include no fertilizer, Urea in low nitrogen rate (Urea low), biofertilizer low, Calcium Nitrate low, Urea:biofertilizer low, Calcium Nitrate (CN):biofertilizer low, Urea in high nitrogen rate (Urea high), biofertilizer high, Urea:biofertilizer high, and Calcium Nitrate (CN):biofertilizer high. Organically Amended Brownfield Soil (FIG. 13A) and Fertilized Brownfield Soil (FIG. 13B) were analyzed.

FIG. 14 shows leaching trials comparing cumulative Ammonium-N leached over time from soil columns treated with synthetic fertilizers, biofertilizer (BF) of the present disclosure, or blends of both. The applications include no fertilizer, Urea in low nitrogen rate (Urea low), biofertilizer low, Calcium Nitrate low, Urea:biofertilizer low, Calcium Nitrate (CN):biofertilizer low, Urea in high nitrogen rate (Urea high), biofertilizer high, Urea:biofertilizer high, and Calcium Nitrate (CN):biofertilizer high.

FIG. 15 shows leaching trials comparing Urea-N leached over time from Organically Amended Brownfield soil columns treated with synthetic fertilizers, biofertilizer (BF) of the present disclosure, or blends of both. The applications include no fertilizer, Urea in low nitrogen rate (Urea low), biofertilizer low, Calcium Nitrate low, Urea:biofertilizer low, Calcium Nitrate (CN):biofertilizer low, Urea in high nitrogen rate (Urea high), biofertilizer high, Urea:biofertilizer high, and Calcium Nitrate (CN):biofertilizer high.

FIGS. 16A, 16B and 16C show leaching trials comparing Urea-N leached over time in soil incubation experiments in which the soil was treated with synthetic fertilizers, biofertilizer (BF) of the present disclosure, or blends of both. The applications include combinations of Fertilizer (F) or Organically Amended (O) soil in combination with calcium nitrate and urea, the biofertilizer (BF) of the present disclosure and calcium nitrate, BF and urea, BF alone, or urea alone. FIG. 16A shows dynamically leached Urea-N in Brownfield Soil. FIG. 16B shows dynamically leached Urea-N in Greenville soil. FIG. 16C shows dynamically leached Urea-N in Lakeland soil.

FIGS. 17A, 17B and 17C show leaching trials comparing dynamically leached Ammonium-N in soil incubation experiments in which Brownfield Soil (FIG. 17A), Greenville Soil (FIG. 17B), or Lakeland Soil (FIG. 17C) were treated with synthetic fertilizers, biofertilizer (BF) of the present disclosure, or blends of both. The applications include combinations of Fertilizer (F) or Organically Amended (O) soil in combination with calcium nitrate and urea, the biofertilizer (BF) of the present disclosure and calcium nitrate, BF and urea, BF alone, or urea alone.

FIGS. 18A, 18B and 18C show leaching trials comparing dynamically leached Nitrate-N in soil incubation experiments in which Brownfield Soil (FIG. 18A), Greenville Soil (FIG. 18B), or Lakeland Soil (FIG. 18C) were treated with synthetic fertilizers, biofertilizer (BF) of the present disclosure, or blends of both. The applications include combinations of Fertilizer (F) or Organically Amended (O) soil in combination with calcium nitrate and urea, the biofertilizer (BF) of the present disclosure and calcium nitrate, BF and urea, BF alone, or urea alone.

FIGS. 19A, 19B, 19C, 19D, 19E and 19F show leaching trials comparing dynamically leached Total Nitrogen and Recover % in soil incubation experiments in which Brownfield Soil (FIG. 19A and FIG. 19B), Greenville Soil (FIG. 19C and FIG. 19D), or Lakeland Soil (FIG. 19E and FIG. 19F) were treated with synthetic fertilizers, biofertilizer (BF) of the present disclosure, or blends of both. The applications include combinations of Fertilizer (F) or Organically Amended (O) soil in combination with calcium nitrate and urea, the biofertilizer (BF) of the present disclosure and calcium nitrate, BF and urea, BF alone, or urea alone.

FIGS. 20A, 20B and 20C show trials comparing pH in soil incubation experiments in which Brownfield Soil (FIG. 20A), Greenville Soil (FIG. 20B), or Lakeland Soil (FIG. 20C) were treated with synthetic fertilizers, biofertilizer (BF) of the present disclosure, or blends of both. The applications include combinations of Fertilizer (F) or Organically Amended (O) soil in combination with calcium nitrate and urea, the biofertilizer (BF) of the present disclosure and calcium nitrate, BF and urea, BF alone, or urea alone.

FIG. 21 shows biological nitrogen fixation rates measured in trial 4 shown by growth period (x-axis) and addition rate (facets). Bars represent average biological nitrogen fixation (μg N fixed g⁻¹ dry substrate day⁻¹)±standard error. Significant differences (p<0.05) between addition treatments are represented by lower case letters. Biological nitrogen fixation rates did not differ significantly by growth period within addition treatments.

FIG. 22 shows background corrected biological nitrogen fixation rates measured in trial 4 shown by growth period (x-axis) and addition rate (facets). Background correction was performed by subtracting the average biological nitrogen fixation value from 0:0 treatments from each value of the remaining treatments. Bars represent average biological nitrogen fixation (μg N fixed g⁻¹ dry substrate day⁻¹)±standard error. Significant differences (p<0.05) between addition treatments are represented by lower case letters. No significant differences in biological nitrogen fixation rates were observed by growth period or addition treatments.

FIGS. 23A and 23B show biological nitrogen fixation rates measured in trial 4 by addition rate (x-axis) averaged across growth period. FIG. 23A shows biological nitrogen fixation from all treatments. FIG. 23B shows background corrected biological nitrogen fixation rates. Points represent average biological nitrogen fixation (μg N fixed g⁻¹ dry substrate day⁻¹)±standard error. Significant differences (p<0.05) between addition treatments are represented by lower case letters.

FIG. 24 shows biological nitrogen fixation rates reported per CFU and shown by addition rate (x-axis). Points represent average BNF (fg N fixed CFU⁻¹ day⁻¹)±standard error. Significant differences (p<0.05) between addition treatments are represented by lower case letters.

FIGS. 25A and 25B show biological nitrogen fixation rates (μg N fixed g⁻¹ dry substrate day⁻¹) of microbes grown in both sterile and non-sterile media, both raw data (FIG. 25A) and background corrected (FIG. 25B). Bars represent average biological nitrogen fixation (μg N fixed g⁻¹ dry substrate day⁻¹)±standard error. Test conditions included two controls (0:0 or 0:20 of BF %:N fertilizer %) and five microbes such as A. brasilense (AB), A. vinelandii (AV), P. polymyxa (PP), S. azotifigens (SA) and biofertilizer (BF) of the present disclosure. FIG. 25B shows background corrected biological nitrogen fixation rates. Background correction was performed by subtracting the average biological nitrogen fixation value (obtained from 0:0 treatments) from each value of the remaining treatments.

FIG. 26 shows response ratios of non-sterile coconut coir media to sterilized coconut coir media for biological nitrogen fixation rates of each organism. The dashed line indicates a 1:1 ratio at which BNF rates of any organism would be equal in non-sterile and sterile media. Values below the 1:1 link indicate BNF rates are greater in sterile relative to non-sterile media. Five microbes such as A. brasilense (AB), A. vinelandii (AV), P. polymyxa (PP), S. azotifigens (SA) and biofertilizer (BF) of the present disclosure were analyzed.

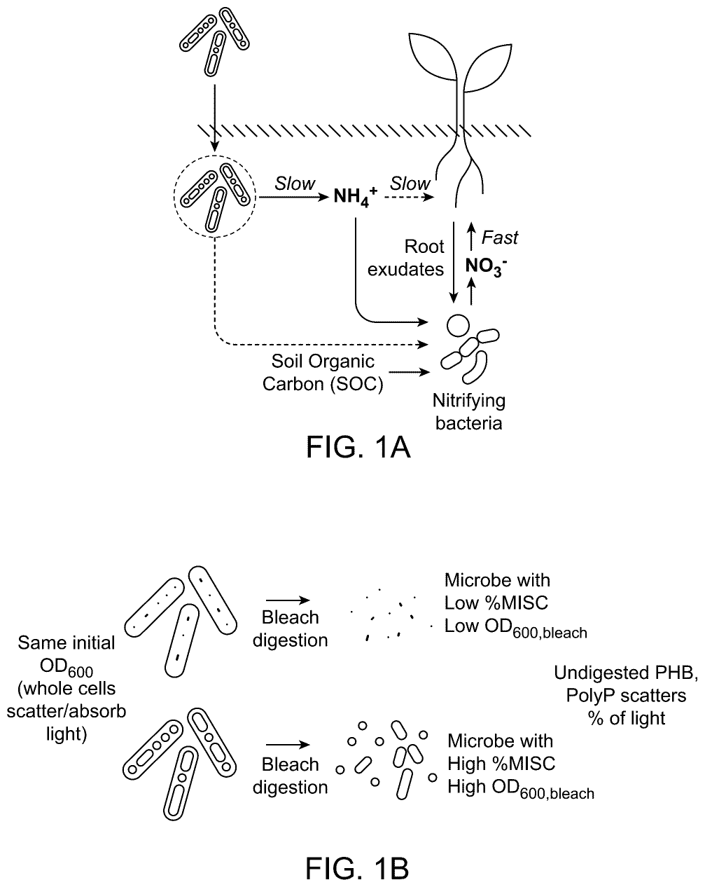

### DETAILED DESCRIPTION OF THE DISCLOSURE

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference. Incorporation by reference of any such documents shall not be considered an admission that the incorporated materials are prior art to the present disclosure, or considered as material to the patentability of the present disclosure.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean about, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “wt %” or “weight/volume” as used herein refers to the ratio between to components with respect to volume. For example, a 5 wt % ethanol in water solution would represent a solution comprising 5 g ethanol for every 100 mL water.

As used herein, the term “medium,” or “media” is used in a broad sense, and refers to and encompasses a variety of solutions, buffers, formulations, and/or compounds, in which a microorganism or other type of biological samples or materials may reside for any period, of time that is conducive to the preservation of viability of the biological material placed within such buffers, solutions, formulations, and/or compounds.

As used herein, the term “carbon source” refers to one or more carbon-containing compounds capable of being metabolized by a microorganism. For example, the medium can include any carbon source suitable for maintaining the viability or growing the microorganism. In some aspects, the carbon source can be an autotrophic substrate. In some aspects, the carbon source can be a heterotrophic substrate. Exemplary carbon sources include, but are not limited to, glucose, succinate, butyrate, pyruvate, propanol, acetate, fructose, sucrose, methanol, butanol, propanol, or mixtures or derivatives thereof. Other exemplary heterotrophic carbon sources include glucose, succinate, butyrate, pyruvate, propanol, acetate, fructose, sucrose, butanol, propanol, ethanol, pentanol, rich broth, L-amino acids, arabinose, sucrose, dextrin, dextrose, lactose, maltose, fructose, galactose, mannose, saccharose, xylose, or mixtures or derivatives thereof.

As used herein, the term “buffer” refers to aqueous solutions or compositions that resist changes in pH when acids or bases are added to the solution. Solutions that exhibit buffering activity are often referred to as “buffers” or “buffer solutions.” Buffers typically are able to maintain the pH of the solution within defined ranges, often for example between pH 4 and pH 9. Exemplary biological buffers include, but are not limited to, phosphate buffer, Lactated Ringer's solution, physiological saline solution, N-(2-Acetamido)-2-aminoethanesulfonic acid (ACES); N-2-acetamido-2-iminodiacetic acid (ADA); N,N-bis(2-hydroxyethyl)-2-aminoethanesulfonic acid (BES); N,N-bis(2-hydroxyethyl)glycine (BICINE); 2-bis(2-hydroxyethyl)amino-2-(hydroxymethyl)-1,3-propanediol (BIS-TRIS); 3-(cyclo hexylamino)-1-propanesulfonic acid (CAPS); 3-(cyclohexylamino)-2-hydroxy-1-propanesulfonic acid (CAPSO); 2-(cyclohexylamino) ethanesulfonic acid (CHES); (N,N-bis[2-hydroxyethyl]amino)-2-hydroxypropanesulfonic acid (DIPSO); 4-(2-Hydroxyethyl)-1-piperazinepropanesulfonic acid (EPPS); 4-(2-hydroxyethyl)piperazine-1-ethanesulfonic acid (HEPES); 4-(2-hydroxyethyl)piperazine-1-(2-hydroxypropane sulfonic acid) (HEPPSO); 2-(N-morphilino)ethanesulfonic acid (MES); 3-(N-morpholino) propanesulfonic acid (MOPS); 3-morpholino-2-hydroxypropanesulfonic acid (MOPSO); piperazine-1,4-bis(2-ethanesulfonic acid) (PIPES); piperazine-N,N′-bis(2-hydroxypropane sulfonic acid) (POPSO); [(2-hydroxy-1,1-bis(hydroxy methyl)ethyl)amino]-1-propanesulfonic acid (TAPS); 3-(N-tris[hydroxymethyl]methylamino)-2-hydroxypropanesulfonic acid (TAPSO); 2-[(2-hydroxy-1,1-bis(hydroxylmethyl)ethyl)amino]ethanesulfonic acid (TES); N-[tris(hydroxymethyl)methyl]glycine (TRICINE); and tris(hydroxymethyl)amino methane) (TRIS); mixtures or derivatives thereof, as well as other biological buffers including those developed by Good, N. E., et al. (1966, Hydrogen Ion Buffers for Biological Research. Biochemistry 5(2), 467-477).

As used herein, the term “effective amount” in terms of a biofertilizer will depend upon a variety of factors, including, for example, percent viability of cells in the biofertilizer, concentration of cells in the biofertilizer, the levels of nutrients, including ammonia and carbon sources (e.g., PHB), and whether the biofertilizer is in the form of a liquid cell suspension or comprises a solid biomass component, such as soils, plant materials, or inert materials. A person of ordinary skill in the art will be able to determine an effective amount taking into account these variables. For purposes of the instant disclosure, an effective amount of a biofertilizer means an amount of the biofertilizer that is sufficient to result in an enhanced property or characteristic of a soil microbiome and/or a crop or plant that is statistically greater than the same property or characteristic in the absence of the biofertilizer, such as, for example, increased crop yield, increased fruit or vegetable yield or root storage mass, increased carbon and/or nitrogen availability in the microbiome. In some aspects, the property or characteristic (e.g., crop or plant yield or yield quality) enhanced by the biofertilizer is observed with at least a 5%, or at least a 6%, or 7%, or 8%, or 9%, or 10%, or 25%, or 50%, or 75%, or 100%, or 200%, or 300%, or 400%, or 500%, or 1000%, or 1250%, or 1500%, or 2000%, or more increase over the same property or characteristic established in the absence of the biofertilizer.

As used herein, the term “microbiome” refers to the collection of all microorganisms living in a particular environment, including in the soil surrounding and/or interacting with the root of a plant.

As used herein, the term “biofertilizer” refers to a preparation containing living cells or latent cells of microorganisms that help plants (e.g., crop plants) grow. The term also refers to a preparation containing living cells or latent cells of microorganisms that help to feed and/or enhance the soil microbiome. The term also refers to a preparation containing living cells or latent cells of microorganisms that produce chemicals (including but not limited to nitrate, ammonia, phosphorus) to directly or indirectly provide nutrition to plants (e.g., crop plants), or to directly or indirectly signal plant or microbial pathways to the benefit of the plant (e.g. crop plants), in the soil, soilless substrate, or other growth medium. In some embodiments, the biofertilizer can be a preparation containing living cells or latent cells of microorganisms, or having enhanced accumulation of MISC, or a combination thereof. In some aspects, the MISC accumulated by the microorganism comprises a polyhydroxyalkanoate (PHA), a polyphosphate (PolyP), a lipid, or a combination thereof. In some aspects, the MISC is a PHA. In some aspects, the PHA is polyhydroxybutyrate (PHB), poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB. In some aspects, the accumulation of MISC, such as PHB, is greater than about 10%, greater than about 12%, greater than about 15%, greater than about 20%, greater than about 25%, greater than about 30%, greater than about 35%, or greater than about 40%.

As used herein, the term “nitrogenase” refers to an enzyme that is produced by certain specialized bacteria called nitrogen-fixing bacteria, such as cyanobacteria and Xanthobacter (e.g., X autotrophicus), which are responsible for reducing atmospheric nitrogen (N₂) to ammonia (NH₃) as part of the nitrogen cycle.

As used herein, the term “sparingly soluble” refers to a chemical entity having a solubility in water, oil, a solvent or a solution less than 1 mg/mL, and particularly less than 0.1 mg/mL.

As used herein, the term “soluble” refers to a chemical entity having a solubility in water, oil, a solvent or a solution at least 1 mg/mL.

As used herein, the term “salt” refers to any chemical compound formed from the reaction of an acid with a base, with all or part of the hydrogen of the acid replaced by a metal or other cation. Examples of salts include salts having a cation as a counterion, such as an alkaline metal ion (e.g., Na⁺, K⁺, etc.), an alkaline earth metal ion (e.g., Mg²⁺, Ca²⁺, etc.), ammonium ion (e.g., NH₄⁺, or an organic ammonium ion), etc. Examples of salts also include salts having an anion as a counterion, such as an inorganic anion (e.g., Cl⁻, SO₄²⁻, Br⁻, HSO₄⁻, etc.) or an organic anion (e.g., a carboxylic acid anion such as a formate, acetate, etc.). As used herein, the term “hydrate” refers to a compound formed by the union of water with some other substance.

As used herein, the term “constituent” or “constituent component” refers to any compound that the nutrient compound source comprises. For example, the constituent components of ammonium magnesium phosphate (MgNH₄PO₄) are ammonium (NH₄⁺), magnesium (Mg²⁺), or phosphate (H₂PO₄⁻, HPO₄²⁻, PO₄³⁻), or a combination thereof.

As used herein, the terms “component,” “nutrient,” “ingredient,” “nutrient component,” and “component nutrient” are used interchangeably and all refer to compounds that can be used in cell culture media to maintain or promote proliferation of cell growth, whether of chemical or biological origin. Components that facilitate or maintain cell culture in vitro can be selected by those skilled in the art within the scope of the present disclosure, depending on the particular needs.

As used herein, the term “solution” or “mixture” refers to a homogenous mixture formed by the process that a solid, a liquid, or a gaseous substance, or a combination thereof is homogeneously mixed with a liquid or sometimes a gas or solid.

As used herein, the term “solubility” refers to the amount of a substance that will dissolve in a given amount of another substance.

As used herein, the term “solubility product” or “solubility product constant” refers to the product of the concentration of the ions that are present in a saturated solution of an ionic compound. The term can be used to describe saturated solutions of ionic compounds of relatively low solubility. A saturated solution is in a state of dynamic equilibrium between the dissolved, dissociated, ionic compound and the undissolved solid.

As used herein, the term “stable” or “stability” refers to the viability of the microorganisms in biofertilizers. Biofertilizers comprise living microorganisms, unlike chemical fertilizers. Biofertilizers themselves can comprise the source of nutrients, and/or can help the crops or plants in accessing the nutrient available in its surrounding environment. The viability of the microorganisms during production, formulation, storage, transportation, distribution and field application is directly related to the performance and potentials of a biofertilizer. A range of commercial biofertilizer formulation strategies can be applied to ensure maximum viability of the microorganisms used in such formulations. These strategies include: (i) optimization of biofertilizer formulation, (ii) application of thermo-tolerant/drought-tolerant/genetically modified strains and, (iii) application of liquid biofertilizer. For convenience of application, a carrier material can be used as a vehicle for the microorganisms to be used as biofertilizer. Moreover, such materials can have a role in maintaining the viability (shelf-life) of the microorganisms prior to its release into the field as well as they also provide a suitable microenvironment for rapid growth of the organisms upon their release. A carrier can be a material, such as peat, vermiculite, lignite powder, clay, talc, rice bran, seed, rock phosphate pellet, charcoal, soil, paddy straw compost, wheat bran or a mixture of such materials. In common practice, for a better shelf-life of a biofertilizer formulation, a carrier or a mixture of carrier materials can be selected based on the viability of the microorganisms mixed with them. Similarly, pre-sterilization of the carrier material and its enrichment with nutrients can be another strategy for improving the shelf-life by allowing the microorganism to maintain and grow in a non-competitive microenvironment. Sucrose, maltose, trehalose, molasses, glucose, and/or glycerol can be supplementary nutrients or cell protectants commonly used with a carrier material to ensure maximum cell viability and extended shelf-life. Liquid biofertilizer formulations can be considered as one potential strategy for improving the shelf-life of biofertilizer. Unlike solid carrier based biofertilizers, liquid formulations allow the manufacturer to include sufficient amount of nutrients, cell protectant, and inducers responsible for cell/spore/cyst formation to ensure prolonged shelf-life. The shelf-life of common solid carrier based biofertilizers can be around six months; however, it could be as high as two years for a liquid formulation. Further, solid carrier based biofertilizers can be less thermo-tolerant, whereas liquid formulations can tolerate the temperature as high as 55° C. Hence, improved shelf-life can be achieved formulating biofertilizers into liquid formulations.

As used herein, the term “% PHB,” “% PHA,” “% PHV”, or “% MISC” refers to the percent intracellular storage compounds over total cell weight. It can be measured by the optical density of the bleach digested sample compared against the optical density of the total undigested sample.

As used herein, the term “struvite” refers to magnesium ammonium phosphate hexahydrate (MgNH₄PO₄·6H₂O). Struvite is a white crystalline substance consisting of equal molar amounts of magnesium, ammonium and phosphate, as well as six water of hydration.

As used herein, the term “initial culture,” “fed-batch cell culture,” or “fed-batch culture” refers to cells (e.g., microorganism cells) and media that are first supplemented to a culture vessel and additional culture nutrients are added during cultivation. The term also refers to a cell culture that is fed continuously or in discrete increments and where cells and/or products are periodically collected or not collected prior to the end of the culture.

As used herein, the term “culture vessel” refers to a glass, plastic, or metal container that can provide a contained environment for culturing the microorganism. The vessel can be a standalone container or part of a larger system or setup.

As used herein, the term “inoculation” refers to the addition of seeding cells to a medium to initiate culture.

As used herein, the term “feed” refers to any addition of any substance made to the culture after inoculation. Feeding can be one or more additions.

As used herein, the term “feed solution,” “feed medium,” or “feed medium” refers to a medium containing one or more nutrients added to a culture that is initiated at some time after inoculation.

As used herein, the term “growth phase” or “proliferative phase” refers to a period in which cultured cells rapidly divide and increase in number. During the proliferative phase, cells can generally be cultured under media and conditions designed to maximize cell growth.

### Compound Nutrient Source

The disclosure relates to a growth medium for a nitrogen-fixing microorganism comprising a compound nutrient source, a salt, a buffer, a carbon source, and a metal source. In one aspect, the compound nutrient source comprises two or more constituent components. In another aspect, the salt comprises a soluble salt of the constituent component. In another aspect, the compound nutrient source has a solubility lower than the solubility of the soluble salt of the constituent component. In another aspect, the concentration of the constituent component salt is below the toxicity level of the nitrogen-fixing microorganism.

A compound nutrient source can be a phase-separated chemical compound of the general form:

AₐB_{b}C_{c}·nH₂O [symbol] aA+bB+cC+nH₂O,

where A can be represented by an ion selected from the following non-limiting list: lithium (1+), sodium (1+), potassium (1+), rubidium (1+), cesium (1+), francium (1+), silver (1+), mercury (1+), thallium (1+), barium (2+), beryllium (2+), cadmium (2+), calcium (2+), chromium (2+), cobalt (2+), copper (2+), europium (2+), gadolinium (2+), germanium (2+), iron (2+), lanthanum (2+), lead (2+), magnesium (2+), manganese (2+), mercury (2+), nickel (2+), osmium (2+), platinum (2+), ruthenium (2+), strontium (2+), tin (2+), uranium (2+), vanadium (2+), yttrium (2+), zinc (2+), cerium (3+), praseodymium (3+), neodymium (3+), promethium (3+), samarium (3+), europium (3+), gadolinium (3+), terbium (3+), dysprosium (3+), holmium (3+), erbium (3+) thulium (3+), ytterbium (3+), lutetium (3+), americium (3+), curium (3+), berkelium (3+), californium (3+), einsteinium (3+), fermium (3+), mendelevium (3+), lawrencium (3+), scandium (3+), yttrium (3+), lanthanum (3+), actinium (3+), chromium (3+), iron (3+), ruthenium (3+), cobalt (3+), rhodium (3+), iridium (3+), gold (3+), boron (3+), aluminum (3+), gallium (3+), indium (3+), thallium (3+), antimony (3+), or bismuth (3+). B can be represented by an ion selected to the following non-limiting list: methylammonium, guanidinium, ammonium, phosphonium, hydronium, hydron, fluoronium, pyrylium, tropylium, Triphenylcarbenium, or cyclopropenium. C can be represented by an ion selected from the following non-limiting list: chloride, hypochlorite, chlorite, chlorate, perchlorate, bromide, hypobromite, bromite, bromate, perbromate, iodide, hypoiodite, iodite, iodate, periodate, sulfide, hyposulfite, sulfite, sulfate, persulfate, selenide, hyposelenite, selenite, selenate, telluride, hypotellurite, tellurite, tellurate, nitride, hyponitrite, nitrite, nitrate, phosphide, hypophosphite, phosphite, phosphate, perphosphate, arsenide, hypoarsenite, arsenite, arsenate, tetrahydroxyborate, acetylide, ethoxide or ethanolate, acetate or ethanoate, benzoate, citrate, carbonate, oxalate, cyanide, chromate, dichromate, bicarbonate or hydrogencarbonate, hydrogen phosphate, dihydrogen phosphate, hydrogen sulfate or bisulfate, manganate, permanganate, azanide or amide, peroxide, hydroxide, bisulfide, thiocyanate, silicate, or thiosulfate; a, b, and c represent the stoichiometric coefficients of each of the respective ions such that the charge balances in the ternary compound, and n represents the stoichiometric amount of water in the crystal that can be in an anhydrous form or in a form of any level of hydrate.

In some aspects, the compound nutrient source comprises ammonium (NH₄⁺), magnesium (Mg²⁺), and phosphate (H₂PO₄, HPO₄²⁻, or PO₄³⁻). In some aspects, the compound nutrient source is selected from ammonium magnesium phosphate (MgNH₄PO₄), or a hydrate thereof. In some aspects, the compound nutrient source is selected from struvite, dittmarite, ammonium magnesium phosphate, ammonium magnesium phosphate hydrate, ammonium magnesium phosphate hexahydrate, magnesium ammonium phosphate, magnesium ammonium phosphate hydrate, magnesium ammonium phosphate hyxahydrate, MAP, or an equivalent thereof.

In some aspects, the compound nutrient source is ammonium magnesium phosphate hexahydrate (MgNH₄PO₄·6H₂O). In one aspect, the compound nutrient source is struvite. In another aspect, the compound nutrient source can be made from premixing ammonium dihydrogen phosphate ((NH₄)H₂PO₄), and magnesium hydroxide (Mg(OH)₂).

In some aspects, the constituent component salt is selected from a soluble salt of ammonium (NH₄⁺), magnesium (Mg²⁺), or phosphate (H₂PO₄⁻, HPO₄²⁻, PO₄³⁻), or a combination thereof. In some aspects, the medium comprises one of each of the soluble salts of ammonium (NH₄⁺), magnesium (Mg²⁺), and phosphate (H₂PO₄⁻, HPO₄²⁻, PO₄³⁻). In some aspects, the medium comprises potassium phosphate monobasic (K₂HPO₄), potassium phosphate dibasic (KH₂PO₄), magnesium sulfate heptahydrate (MgSO₄·7H₂O), or ammonium sulfate ((NH₄)₂SO₄), or a combination thereof.

In some aspects, the concentration of ammonium is less than about 20 mM. In one aspect, the concentration of ammonium is from about 5 mM to about 12 mM. In one aspect, the concentration of ammonium is about 5 mM. In one aspect, the concentration of ammonium is about 6 mM. In one aspect, the concentration of ammonium is about 7 mM. In one aspect, the concentration of ammonium is about 8 mM. In one aspect, the concentration of ammonium is about 9 mM. In one aspect, the concentration of ammonium is about 10 mM. In one aspect, the concentration of ammonium is about 11 mM. In one aspect, the concentration of ammonium is about 12 mM. In some aspects, the concentration of ammonium is less than about 20 mM, less than about 19 mM, less than about 18 mM, less than about 17 mM, less than about 16 mM, less than about 15 mM, less than about 14 mM, less than about 13 mM, less than about 12 mM, less than about 11 mM, less than about 10 mM, less than about 9 mM, less than about 8 mM, less than about 7 mM, less than about 6 mM, or less than about 5 mM. In some aspects, the concentration of ammonium ranges from about 5 mM to about 10 mM, from about 5 mM to about 12 mM, from about 5 mM to about 15 mM, from about 10 mM to about 15 mM, from about 5 mM to about 20 mM, from about 10 mM to about 20 mM, or from about 15 mM to about 20 mM.

In some aspects, the compound nutrient source further comprises metal carbonate. In some aspects, the metal carbonate comprises a divalent cationic metal.

Metal carbonates can take a general form (M)CO₃·nH₂O, where M is a divalent cationic metal. M can be represented by an ion selected from the following non-limiting list: barium (2+), beryllium (2+), cadmium (2+), calcium (2+), chromium (2+), cobalt (2+), copper (2+), europium (2+), gadolinium (2+), germanium (2+), iron (2+), lanthanum (2+), lead (2+), magnesium (2+), manganese (2+), mercury (2+), nickel (2+), osmium (2+), platinum (2+), ruthenium (2+), strontium (2+), tin (2+), uranium (2+), vanadium (2+), yttrium (2+), or zinc (2+). n represents the stoichiometric amount of water in a crystal that can be in an anhydrous form or in a form of any level of hydrate.

Similarly to magnesium ammonium phosphate and other compound nutrient sources, in some aspects, metal carbonates can exist and function as a compound nutrient source in the form M′ₓM″₁₋ₓCO₃·nH₂O, where M′ and M″ are both divalent cationic metals. M′ and M″ can be represented by two distinct ions selected from the following non-limiting list: barium (2+), beryllium (2+), cadmium (2+), calcium (2+), chromium (2+), cobalt (2+), copper (2+), europium (2+), gadolinium (2+), germanium (2+), iron (2+), lanthanum (2+), lead (2+), magnesium (2+), manganese (2+), mercury (2+), nickel (2+), osmium (2+), platinum (2+), ruthenium (2+), strontium (2+), tin (2+), uranium (2+), vanadium (2+), yttrium (2+), or zinc (2+). n represents the stoichiometric amount of water in a crystal that can be in an anhydrous form or in a form of any level of hydrate.

In other aspects, the metal carbonates can exist with a monovalent ion and a trivalent ion selected from the following non-limiting list: lithium (1+), sodium (1+), potassium (1+), rubidium (1+), cesium (1+), francium (1+), silver (1+), mercury (1+), thallium (1+), cerium (3+), praseodymium (3+), neodymium (3+), promethium (3+), samarium (3+), europium (3+), gadolinium (3+), terbium (3+), dysprosium (3+), holmium (3+), erbium (3+) thulium (3+), ytterbium (3+), lutetium (3+), americium (3+), curium (3+), berkelium (3+), californium (3+), einsteinium (3+), fermium (3+), mendelevium (3+), lawrencium (3+), scandium (3+), yttrium (3+), lanthanum (3+), actinium (3+), chromium (3+), iron (3+), ruthenium (3+), cobalt (3+), rhodium (3+), iridium (3+), gold (3+), boron (3+), aluminum (3+), gallium (3+), indium (3+), thallium (3+), antimony (3+), or bismuth (3+). These ions can replace either M′ and/or M″, provided that the stoichiometric coefficients are adjusted to balance charge in the metal carbonate compound.

Metal carbonates comprising more than two metals can also be formulated following the pattern discussed herein. In some aspects, the metal carbonates comprise two, three, four, five, six, or more metals.

In some aspects, the divalent cationic metal comprises Ca²⁺, Mg²⁺, Fe²⁺, Ni²⁺, or Co²⁺, or a combination thereof. In one aspect, the divalent cation metal is Fe²⁺. In another aspect, the medium comprises ferric citrate and ammonium bicarbonate ((NH₄)HCO₃).

### Culture Compositions

The disclosure also relate to the aspects of culture compositions comprising the microorganisms. In some aspects, the culture medium further comprises a soluble salt of calcium (Ca²⁺). In one aspect, the salt is CaSO₄.

In some aspects, the medium comprises a carbon source. In one aspect, the carbon source is selected from an autotrophic carbon source, or a heterotrophic carbon source, or a combination thereof. In one aspect, the carbon source is an autotrophic carbon source. In one aspect, the autotrophic carbon source is methanol. In another aspect, the carbon source is a heterotrophic carbon source.

In some aspects, the carbon source is selected from a carbohydrate (such as monosaccharide, disaccharide, oligosaccharide, or polysaccharides), invert sugar (e.g., enzymatically treated sucrose syrup), glycerol, glycerine (e.g., a glycerine byproduct of a biodiesel or soap-making process), dihydroxyacetone, one-carbon source, oil (e.g., a plant or vegetable oil such as corn, palm, or soybean oil), animal fat, animal oil, fatty acid (e.g., a saturated fatty acid, unsaturated fatty acid, or polyunsaturated fatty acid), lipid, phospholipid, glycerolipid, monoglyceride, diglyceride, triglyceride, polypeptide (e.g., a microbial or plant protein or peptide), renewable carbon source (e.g., a biomass carbon source such as a hydrolyzed biomass carbon source), yeast extract, component from a yeast extract, polymer, acid, alcohol, aldehyde, ketone, amino acid, succinate, lactate, acetate, or ethanol, or a combination thereof. Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose).

In some aspect, the medium comprises, in addition to a carbohydrate, a carbon source other than a carbohydrate (e.g., glycerol, glycerine, dihydroxyacetone, one-carbon source, oil, animal fat, animal oil, fatty acid, lipid, phospholipid, glycerolipid, monoglyceride, diglyceride, triglyceride, renewable carbon source, or a component from a yeast extract). In some aspects, the medium comprises, in addition to a carbohydrate, a polypeptide (e.g., a microbial or plant protein or peptide). In some aspects, the microbial polypeptide is a polypeptide from yeast or bacteria. In some aspects, the plant polypeptide is a polypeptide from soy, corn, canola, jatropha, palm, peanut, sunflower, coconut, mustard, rapeseed, cottonseed, palm kernel, olive, safflower, sesame, or linseed.

In some aspects, the cells can be cultured in a medium comprising an uncommon carbon source, which can be selected from the group consisting of glycerol, cell mass, protein, alcohol (e.g., methanol, ethanol, etc.), plant-derived oil, and any combination thereof. In some aspects, the carbon source can be synthesized from carbon dioxide.

In some aspects, the carbon source can be an organic acid. In one aspect, the carbon source can be succinate. In other aspects, the carbon source can be a heterotrophic carbon source. In one aspect, the carbon source can be fructose, or sucrose, or a combination thereof.

In one aspect, the carbon source is an autotrophic carbon source. In one aspect, the autotrophic carbon source is methanol. Methanol is also termed as pseudo-autotropic carbon source because it is catabolized unusually by initial oxidation to CO₂, whereby it is up-taken through the regular autotrophic pathway (RuBisCO).

In some aspects, the medium comprises a metal source. In some aspects, the metal source comprises a trace metal or semi-metal source. In one aspect, the trace metal or semi-metal source is ferric citrate, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), or nickel (II) sulfate hexahydrate (NiSO₄·6H₂O), or a combination thereof. In one aspect, the combination includes ferric citrate, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O).

In some aspects, the medium has a pH ranging from about 5.5 to about 6.5. In some aspects, the medium has a pH of about 6.8. In some aspects, the medium is formulated at a specific pH, and the pH of the medium changes through the course of the bacterial growth. In some aspects, the medium is formulated at a pH of about 6.8. In some aspects, the pH drops to a range of from about 5.5 to about 6.5.

In some aspects, the medium has a pH ranging from about 4.0 to about 9.0.

In some aspects, the medium has a pH ranging from about 4.0 to about 4.5, from about 4.0 to about 5.0, from about 4.0 to about 5.5, from about 4.0 to about 6.0, from about 4.0 to about 6.5, from about 4.0 to about 6.8, from about 4.0 to about 7.0, from about 4.0 to about 7.5, from about 4.0 to about 8.0, from about 4.0 to about 8.5, from about 4.0 to about 9.0, from about 4.5 to about 5.0, from about 4.5 to about 5.5, from about 4.5 to about 6.0, from about 4.5 to about 6.5, from about 4.5 to about 6.8, from about 4.5 to about 7.0, from about 4.5 to about 7.5, from about 4.5 to about 8.0, from about 4.5 to about 8.5, from about 4.5 to about 9.0, from about 5.0 to about 5.5, from about 5.0 to about 6.0, from about 5.0 to about 6.5, from about 5.0 to about 6.8, from about 5.0 to about 7.0, from about 5.0 to about 7.5, from about 5.0 to about 8.0, from about 5.0 to about 8.5, from about 5.0 to about 9.0, from about 5.5 to about 6.0, from about 5.5 to about 6.5, from about 5.5 to about 6.8, from about 5.5 to about 7.0, from about 5.5 to about 7.5, from about 5.5 to about 8.0, from about 5.5 to about 8.5, from about 5.5 to about 9.0, from about 6.0 to about 6.5, from about 6.0 to about 6.8, from about 6.0 to about 7.0, from about 6.0 to about 7.5, from about 6.0 to about 8.0, from about 6.0 to about 8.5, from about 6.0 to about 9.0, from about 6.5 to about 6.8, from about 6.5 to about 7.0, from about 6.5 to about 7.5, from about 6.5 to about 8.0, from about 6.5 to about 8.5, from about 6.5 to about 9.0, from about 7.0 to about 7.5, from about 7.0 to about 8.0, from about 7.0 to about 8.5, from about 7.0 to about 9.0, from about 7.5 to about 8.0, from about 7.5 to about 8.5, from about 7.5 to about 9.0, from about 8.0 to about 8.5, from about 8.0 to about 9.0, or from about 8.5 to about 9.0.

In some aspects, the medium has a pH of about 4.0, about 4.5, about 5.0, about 5.5, about 6.0, about 6.5, about 6.8, about 7.0, about 7.5, about 8.0, about 8.5, or about 9.0.

In some aspects, the medium can be buffered by a buffer and/or a compound nutrient source. In some aspects, the pH of the medium can be buffered by the buffer. In some aspects, the suitable buffer can be boric acid, sodium borate, potassium citrate, citric acid, sodium bicarbonate, TRIS, or phosphate buffer. In some aspects, the buffer is phosphate buffer. In some aspects, the buffer is phosphate buffer, Lactated Ringer's solution, physiological saline solution, N-(2-Acetamido)-2-aminoethanesulfonic acid (ACES); N-2-acetamido-2-iminodiacetic acid (ADA); N,N-bis(2-hydroxyethyl)-2-aminoethanesulfonic acid (BES); N,N-bis(2-hydroxyethyl)glycine (BICINE); 2-bis(2-hydroxyethyl)amino-2-(hydroxymethyl)-1,3-propanediol (BIS-TRIS); 3-(cyclo hexylamino)-1-propanesulfonic acid (CAPS); 3-(cyclohexylamino)-2-hydroxy-1-propanesulfonic acid (CAPSO); 2-(cyclohexylamino) ethanesulfonic acid (CHES); (N,N-bis[2-hydroxyethyl]amino)-2-hydroxypropanesulfonic acid (DIPSO); 4-(2-Hydroxyethyl)-1-piperazinepropanesulfonic acid (EPPS); 4-(2-hydroxyethyl)piperazine-1-ethanesulfonic acid (HEPES); 4-(2-hydroxyethyl)piperazine-1-(2-hydroxypropane sulfonic acid) (HEPPSO); 2-(N-morphilino)ethanesulfonic acid (MES); 3-(N-morpholino) propanesulfonic acid (MOPS); 3-morpholino-2-hydroxypropanesulfonic acid (MOPSO); piperazine-1,4-bis(2-ethanesulfonic acid) (PIPES); piperazine-N,N′-bis(2-hydroxypropane sulfonic acid) (POPSO); [(2-hydroxy-1,1-bis(hydroxy methyl)ethyl)amino]-1-propanesulfonic acid (TAPS); 3-(N-tris[hydroxymethyl]methylamino)-2-hydroxypropanesulfonic acid (TAPSO); 2-[(2-hydroxy-1,1-bis(hydroxylmethyl)ethyl)amino]ethanesulfonic acid (TES); N-[tris(hydroxymethyl)methyl]glycine (TRICINE); or tris(hydroxymethyl)amino methane) (TRIS); or mixtures or derivatives thereof; or other biological buffers including those developed by Good, N. E., et al. (1966, Hydrogen Ion Buffers for Biological Research. Biochemistry 5(2), 467-477).

The disclosure also relates to a medium comprising ammonium magnesium phosphate hexahydrate (MgNH₄PO₄·6H₂O), ferric citrate, ammonium bicarbonate ((NH₄)HCO₃), potassium phosphate monobasic (K₂HPO₄), potassium phosphate dibasic (KH₂PO₄), magnesium sulfate heptahydrate (MgSO₄·7H₂O), calcium sulfate dihydrate (CaSO₄·2H₂O), ammonium sulfate ((NH₄)₂SO₄), methanol, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O).

The disclosure further relates to a medium comprising ammonium dihydrogen phosphate ((NH₄)H₂PO₄), magnesium hydroxide (Mg(OH)₂), ferric citrate, ammonium bicarbonate ((NH₄)HCO₃), potassium phosphate monobasic (K₂HPO₄), potassium phosphate dibasic (KH₂PO₄), magnesium sulfate heptahydrate (MgSO₄·7H₂O), calcium sulfate dihydrate (CaSO₄·2H₂O), ammonium sulfate ((NH₄)₂SO₄), methanol, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O).

### Microorganisms

The present disclosure relates to culturing a suitable species, strain, isolated, purified, cultured, fermented, enriched, resuscitated, modified, or genetically engineered microorganism.

In some aspects, the microorganism is a nitrogen-fixing microorganism. In some aspects, the nitrogen-fixing microorganism expresses nitrogenase. In some aspect, the nitrogen-fixing microorganism accumulates a microbial intracellular storage compound (MISC). In some aspects, the nitrogen-fixing microorganism expresses nitrogenase and accumulates a MISC. In some aspects, the MISC comprises a polyhydroxyalkanoate (PHA), a polyphosphate (PolyP), or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

In some aspects, the nitrogen-fixing microorganism in the biofertilizer comprises bacteria. In some aspects, the nitrogen-fixing microorganism is a PHA-producing bacteria. In some aspects, the nitrogen-fixing microorganism is a PHB-producing bacteria. In some aspects, the nitrogen-fixing microorganism is a PHV-producing bacteria.

In other aspects, the nitrogen-fixing microorganism in the biofertilizer comprises archaea. In other aspects, the nitrogen-fixing microorganism in the biofertilizer comprises fungi.

In some aspects, the nitrogen-fixing microorganism in the biofertilizer comprises one or more strains of Acidiphilium species, Acidiphilium multivorum, Alcaligenes species, Alcaligenes paradoxus, Arthrobacter species, Azohydromonas species, Azohydromonas australica, Azohydromonas species, Azohydromonas lata, Azospirillum species, Azospirillum amazonsense, Azospirillum lipoferum, Azospirillum thiophilum, Azotobacter vinelandii, Beggiatoa species, Beggiatoa alba, Beijerinckia species, Beijerinckia mobilis, Bradyrhizobium species, Bradyrhizobium elnakii, Bradyrhizobium japonicum, Bradyrhizobium japonicum (strain USDA 122), Burkholderia species, Burkholderia vietnameiensis, Cupriavidus species, Cupriavidus necator, Derxia species, Derxia gummosa, Herbaspirillum species, Herbaspirillum autrotrophicum, Hydrogenophaga species, Hydrogenophaga pseudoflava, Mesorhizobium species, Mesorhizobium alhagi, Methylibium species, Methylibium petroleiphilum, Methylocapsa species, Methylocapsa aurea, Methyloferula species, Methyloferula stellate, Methyloversatilis species, Methyloversatilis universalis, Microcyclus species, Microcyclus aquaticus, Microcyclus ebruneus, Nitrosococcus species, Nitrosococcus oceani, Nitrosomonas communis, Nitrospirillum amazonense, Nocardia species, Nocardia autotrophica, Nocardia opaca, Oligotropha species, Oligotropha carboxidovorans, Pannonibacter species, Pannonibacter phragmitetus, Paracoccus species, Paracoccus denitrificans, Paracoccus pantrophus, Paracoccus yeei, Pelagibaca species, Pelagibaca bermudensis, Pseudomonas species, Pseudomonas facilis, Pseudooceanicola species, Pseudooceanicola atlanticus, Ralstonia species, Ralstonia eutropha, Renobacter species, Renobacter vacuolatum, Rhizobium species, Rhizobium gallicum, Rhizobium japonicum, Rhodobacter species, Rhodobacter capsulatus, Rhodobacter sphaeroides, Rhodomicrobium species, Rhodomicrobium vannielii, Rubrivivax species, Rubrivivax gelatinosus, Salipiger species, Salipiger mucosus, Sinorhizobium species, Sinorhizobium americanum, Sinorhizobium fredii, Sinorhizobium meliloti, Skermanella species, Skermanella stibiiresistens, Stappia species, Stappia aggregate, Thauera species, Thauera humireducens, Variovorax species, Variovorax paradoxus, Xanthobacter species, or Xanthobacter autotrophicus.

In one aspect, the nitrogen-fixing microorganism in the biofertilizer is Xanthobacter autotrophicus. In another aspect, the nitrogen-fixing microorganism in the biofertilizer is Ralstonia eutropha. In some aspects, the nitrogen-fixing microorganism in the biofertilizer is Azotobacter vinelandii.

In some aspects, the Xanthobacter autotrophicus comprises Xanthobacter autotrophicus DSM 431, Xanthobacter autotrophicus DSM 432, Xanthobacter autotrophicus DSM 597, Xanthobacter autotrophicus DSM 685, Xanthobacter autotrophicus DSM 1393, Xanthobacter autotrophicus DSM 1618, Xanthobacter autotrophicus DSM 2009, Xanthobacter autotrophicus DSM 2267, Xanthobacter autotrophicus DSM 3874, Xanthobacter autotrophicus CCUG 44692, or any other Xanthobacter autotrophicus strain associated with NCBI Taxonomy ID 280.

In some aspects, the Azotobacter vinelandii comprises Azotobacter vinelandii DSM 2289, Azotobacter vinelandii DSM 279, Azotobacter vinelandii DSM 332, Azotobacter vinelandii DSM 366, Azotobacter vinelandii DSM 382, Azotobacter vinelandii DSM 389, Azotobacter vinelandii DSM 390, Azotobacter vinelandii DSM 395, Azotobacter vinelandii DSM 399, Azotobacter vinelandii DSM 576, Azotobacter vinelandii DSM 720, Azotobacter vinelandii DSM 2290, Azotobacter vinelandii DSM 85, Azotobacter vinelandii DSM 86, Azotobacter vinelandii DSM 87, Azotobacter vinelandii DSM 13529, Azotobacter vinelandii ATCC 9046, or any other Azotobacter vinelandii strain associated with NCBI Taxonomy ID 354.

In some aspects, the microorganism can naturally possess an MISC accumulation pathway.

Some aspects of the present disclosure include a biofertilizer comprising one or more microorganisms. In some aspects, the biofertilizer comprises a nitrogen-fixing microorganism discussed herein. Some aspects of the present disclosure include a biofertilizer comprising more than one microorganisms. In some aspects, the disclosure relates to a biofertilizer comprising a combination of the nitrogen-fixing microorganism with another microorganism. An often used bacterial group in the combination is rhizobacteria, commonly denominated plant growth promoting rhizobacteria (PGPR). PGPR colonizes plant roots and has several functions such as: nitrogen fixation, phosphorus solubilization, phytohormone production (auxins and cytokinines), production of root-growth promoting volatile compounds (e.g., 2-3-butanediol), nitrogen oxidation from organic sources, siderophores production, among others (Bruto, M., Prigent-Combaret, C., Muller, D. et al. Analysis of genes contributing to plant-beneficial functions in plant growth-promoting rhizobacteria and related Proteobacteria. Sci Rep 4, 6261, 2014). Exemplary but not-limiting rhizobacteria are Azotobacter spp., Bacillus megaterium, Flavobacterium sp., Acetobacter sp., Azospirillum sp., Bacillus thuringiensis, Bacillus subtillis, Arthrobacter globiformis, Arthrobacter agilis, Nocardia coarallina, Pseudomonas fluorescens, Bacteroides succinogenes, Bacteroides lipolyticum, Kurthis zopfil, Brevibacterium lipolyticum, Aspergillus terreus, Rhizopus arrhizus, Azotobacter chroococcum, Azotobacter paspali, Myrothecium verrucaria, Trichoderma viride, Phanerochaete chrysosporium, Pseudomonas halestorga, Pseudomonas calcis, Pseudomonas gelatic, Pseudomonas marinoglutionosa, Pseudomonas nigriaciens, Brevibacterium stationis, Arthrobacter citreus, Arthrobacter luteus, Arthrobacter simplex, Azospirillum brasiliense, Azospirillum lipoferum, Bacillus brenis, Bacillus macerans, Bacillus pumilus, Bacillus polymyxa, Pseudomonas putida, Streptomycus cellulasae, Streptomycus fradiae, Streptomucus griseoflavus, or Acinetobacter lwoffii. Exemplary but non-limiting fungi species is Trichoderma sp.

### MISC

In some aspects, the MISC accumulated by the microorganism comprises a polyhydroxyalkanoate (PHA), a polyphosphate (PolyP), a lipid, or a combination thereof. In some aspects, the MISC is a PHA. In some aspects, the PHA is polyhydroxybutyrate (PHB), poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

A cell tends to store any polymers that can be accumulated without decreasing the rate of growth. The formation of these storage compounds can be promoted in response to stress condition (e.g., nitrogen or phosphorous limitation). The nature of these polymers will depend primarily upon the rate-limiting step in growth and, therefore, upon the nature and level of nutrients in the medium. These polymers, acting as reserves of nutrient and/or energy and including glycogen, PHA and PolyP, would allow the cells to survive periods of inevitable famine. The MISC can be analyzed using spectroscopies (e.g., optical spectroscopy, NMR, FTIR, etc.) and other analytical techniques (e.g., HPLC, HPLC/MS, GC, GC/MS or GC/MS/MS, GPC, DSC, TGA, etc.) (M. B., Galia, Handbook of Hydrocarbon and Lipid Microbiology pp 3725-3741; A. Falvo et al., 2001, Journal of Applied Microbiology).

In some aspects, the accumulation of MISC, such as PHB, is greater than about 10%, greater than about 12%, greater than about 15%, greater than about 20%, greater than about 25%, greater than about 30%, greater than about 35%, or greater than about 40%.

In some aspects, the accumulation of MISC is greater than about 25% to about 40%.

In some aspects, the accumulation of MISC is from about 10% to about 12%, from about 10% to about 15%, from about 10% to about 20%, from about 10% to about 25%, from about 12% to about 15%, from about 12% to about 20%, from about 12% to about 25%, from about 12% to about 30%, from about 15% to about 20%, from about 15% to about 25%, from about 15% to about 30%, from about 15% to about 35%, from about 20% to about 25%, from about 20% to about 30%, from about 20% to about 35%, from about 20% to about 40%, from about 25% to about 30%, from about 25% to about 35%, from about 25% to about 40%, from about 30% to about 35%, from about 30% to about 40%, or from about 35% to about 40%.

In other aspects, the accumulation of MISC is about 10%, about 12%, about 15%, about 20%, about 25%, about 30%, about 35%, or about 40%.

In one aspect, the accumulation of MISC is about 25%. In one aspect, the accumulation of MISC is about 30%. In one aspect, the accumulation of MISC is about 40%.

In some aspects, the accumulation of MISC is measured as the OD₆₀₀ of the culture of the microorganism after bleach digest of the microorganism over the original OD₆₀₀ of the culture of the microorganism.

The disclosed methods of enhancing the accumulation of a MISC in a nitrogen-fixing microorganism comprise a) growing the nitrogen-fixing microorganism in a growth medium comprising a compound nutrient source, a salt, a buffer, a carbon source, and a metal source, and b) adding a carbon source in an amount that exceeds the amount sufficient for the growth of the microorganism, thereby enhance the accumulation of the MISC in the microorganism. In one aspect, the compound nutrient source comprises three or more constituent components. In one aspect, the salt comprises a soluble salt of the constituent component. In one aspect, the compound nutrient source has a solubility lower than the solubility of the soluble salt of the constituent component. In one aspect, the concentration of the constituent component salt is below the toxicity level of the nitrogen-fixing microorganism. In one aspect, the culture of the nitrogen-fixing microorganism is grown to a specified concentration measured by an optical density at 600 nm (OD₆₀₀).

In one aspect, the compound nutrient source is added to the growth medium in a batch mode. In another aspect, the compound nutrient source is added in a fed-batch mode. In one aspect, the compound nutrient source is added using feedback loop system.

In some aspects, the method further comprises inoculating the medium with an initial culture of the nitrogen-fixing microorganism. In some aspects, the medium is inoculated with the initial culture of the nitrogen-fixing microorganism. In some aspects, the medium is inoculated with the initial culture of the nitrogen-fixing microorganism having an OD₆₀₀ greater than about 2. In other aspects, the culture of the microorganism has an OD₆₀₀ greater than about 0, greater than about 2, greater than about 5, greater than about 7, greater than about 10, greater than about 15, greater than about 20, greater than about 25, greater than about 30, greater than about 35, or greater than about 40. In other aspects, the culture of the microorganism culture has an OD₆₀₀ of a range from about 0 to about 40. In other aspects, the culture of the microorganism culture has an OD₆₀₀ from about 0 to about 1, from about 0 to about 2, from about 0 to about 3, from about 0 to about 4, from about 0 to about 5, from about 2 to about 3, from about 2 to about 4, from about 2 to about 5, from about 3 to about 4, from about 3 to about 5, from about 3 to about 7, from about 4 to about 7, from about 4 to about 8, from about 4 to about 10, from about 5 to about 10, from about 5 to about 12, from about 5 to about 15, from about 5 to about 20, from about 7 to about 10, from about 7 to about 12, from about 7 to about 15, from about 7 to about 20, from about 8 to about 10, from about 8 to about 12, from about 8 to about 15, from about 8 to about 20, from about 9 to about 10, from about 9 to about 12, from about 9 to about 15, from about 9 to about 20, from about 10 to about 12, from about 10 to about 15, from about 10 to about 20, from about 12 to about 15, from about 12 to about 20, from about 12 to about 23, from about 12 to about 25, from about 15 to about 20, from about 15 to about 23, from about 15 to about 25, from about 17 to about 20, from about 17 to about 23, from about 17 to about 25, from about 17 to about 27, from about 20 to about 23, from about 20 to about 25, from about 20 to about 27, from about 20 to about 30, from about 23 to about 25, from about 23 to about 27, from about 23 to about 30, from about 23 to about 33, from about 25 to about 27, from about 25 to about 30, from about 25 to about 33, from about 25 to about 35, from about 27 to about 30, from about 27 to about 33, from about 27 to about 35, from about 27 to about 37, from about 30 to about 33, from about 30 to about 35, from about 30 to about 37, from about 30 to about 40, from about 33 to about 35, from about 33 to about 37, from about 33 to about 40, from about 33 to about 43, from about 35 to about 37, from about 35 to about 40, from about 35 to about 43, from about 35 to about 45, from about 37 to about 40, from about 37 to about 43, from about 37 to about 45, from about 40 to about 43, from about 40 to about 45, or from about 43 to about 45. In other aspects, the culture of the microorganism has an OD₆₀₀ of about 0, about 2, about 3, about 4, about 5, about 7, about 8, about 9, about 10, about 12, about 15, about 20, about 23, about 25, about 27, about 30, about 33, about 35, about 37, about 40, about 43, or about 45. In one aspect, the culture of the microorganism has an OD₆₀₀ greater than about 2. In one aspect, the culture of the microorganism has an OD₆₀₀ from about 2 to about 4. In another aspect, the culture of the microorganism has an OD₆₀₀ of about 2. In another aspect, the culture of the microorganism has an OD₆₀₀ greater than about 20. In another aspect, the culture of the microorganism has an OD₆₀₀ of about 20. In one aspect, the culture of the microorganism has an OD₆₀₀ greater than about 25. In another aspect, the culture of the microorganism has an OD₆₀₀ of about 25.

### Formulations

A biofertilizer formulation is critical for its practical applications and performance. A biofertilizer formulation can convert a promising laboratory-proven microorganism, carefully-cultivated by skilled persons in carefully designed and supervised experiments into a commercial product used by common growers under controlled greenhouse or uncontrolled field conditions. A biofertilizer formulation can be designed to deliver repeated positive results, easy storage and handling, long shelf life, and reasonable pricing.

In some aspects, the biofertilizer can be the culture medium itself, or the culture medium formulated in liquid, slurry, granular, or powder dispersal forms. In some aspects, the biofertilizer is formulated to deliver the microorganisms having an accumulation of a MISC greater than about 10%, the growth medium, or the culture of the microorganism, or a combination thereof, to the soil.

In one aspect, the liquid biofertilizer can be formulated to comprise the nitrogen-fixing microorganism in a specified concentration measured by an optical density at 600 nm (OD₆₀₀).

In some aspects, the liquid biofertilizer formulation has an OD₆₀₀ greater than about 2. In other aspects, the liquid biofertilizer formulation has an OD₆₀₀ greater than about 0, greater than about 2, greater than about 5, greater than about 7, greater than about 10, greater than about 15, greater than about 20, greater than about 25, greater than about 30, greater than about 35, or greater than about 40. In other aspects, the liquid biofertilizer formulation has an OD₆₀₀ of a range from about 0 to about 40. In other aspects, the liquid biofertilizer formulation has an OD₆₀₀ from about 0 to about 1, from about 0 to about 2, from about 0 to about 3, from about 0 to about 4, from about 0 to about 5, from about 2 to about 3, from about 2 to about 4, from about 2 to about 5, from about 3 to about 4, from about 3 to about 5, from about 3 to about 7, from about 4 to about 7, from about 4 to about 8, from about 4 to about 10, from about 5 to about 10, from about 5 to about 12, from about 5 to about 15, from about 5 to about 20, from about 7 to about 10, from about 7 to about 12, from about 7 to about 15, from about 7 to about 20, from about 8 to about 10, from about 8 to about 12, from about 8 to about 15, from about 8 to about 20, from about 9 to about 10, from about 9 to about 12, from about 9 to about 15, from about 9 to about 20, from about 10 to about 12, from about 10 to about 15, from about 10 to about 20, from about 12 to about 15, from about 12 to about 20, from about 12 to about 23, from about 12 to about 25, from about 15 to about 20, from about 15 to about 23, from about 15 to about 25, from about 17 to about 20, from about 17 to about 23, from about 17 to about 25, from about 17 to about 27, from about 20 to about 23, from about 20 to about 25, from about 20 to about 27, from about 20 to about 30, from about 23 to about 25, from about 23 to about 27, from about 23 to about 30, from about 23 to about 33, from about 25 to about 27, from about 25 to about 30, from about 25 to about 33, from about 25 to about 35, from about 27 to about 30, from about 27 to about 33, from about 27 to about 35, from about 27 to about 37, from about 30 to about 33, from about 30 to about 35, from about 30 to about 37, from about 30 to about 40, from about 33 to about 35, from about 33 to about 37, from about 33 to about 40, from about 33 to about 43, from about 35 to about 37, from about 35 to about 40, from about 35 to about 43, from about 35 to about 45, from about 37 to about 40, from about 37 to about 43, from about 37 to about 45, from about 40 to about 43, from about 40 to about 45, or from about 43 to about 45. In other aspects, the liquid biofertilizer formulation has an OD₆₀₀ of about 0, about 2, about 3, about 4, about 5, about 7, about 8, about 9, about 10, about 12, about 15, about 20, about 23, about 25, about 27, about 30, about 33, about 35, about 37, about 40, about 43, or about 45. In one aspect, the liquid biofertilizer formulation has an OD₆₀₀ greater than about 2. In one aspect, the liquid biofertilizer formulation has an OD₆₀₀ from about 2 to about 4. In another aspect, the liquid biofertilizer formulation has an OD₆₀₀ of about 2. In another aspect, the liquid biofertilizer formulation has an OD₆₀₀ greater than about 20. In another aspect, the liquid biofertilizer formulation has an OD₆₀₀ of about 20. In one aspect, the liquid biofertilizer formulation has an OD₆₀₀ greater than about 25. In another aspect, the liquid biofertilizer formulation has an OD₆₀₀ of about 25.

In some aspects, the liquid biofertilizer formulation has a colony forming unit (CFU)/mL ranging from about 10² to about 10¹¹ per mL. In some aspects, the liquid biofertilizer formulation has a colony forming unit (CFU)/mL ranging from about 10² to about 10³, from about 10² to about 10⁴, from about 10² to about 10⁵, from about 10² to about 10⁶, from about 10² to about 10⁷, from about 10² to about 10⁸, from about 10² to about 10⁹, from about 10² to about 10¹⁰, from about 10² to about 10¹¹, from about 10³ to about 10⁴, from about 10³ to about 10⁵, from about 10³ to about 10⁶, from about 10³ to about 10⁷, from about 10³ to about 10⁸, from about 10³ to about 10⁹, from about 10³ to about 10¹⁰, from about 10³ to about 10¹¹, from about 10⁴ to about 10⁵, from about 10⁴ to about 10⁶, from about 10⁴ to about 10⁷, from about 10⁴ to about 10⁸, from about 10⁴ to about 10⁹, from about 10⁴ to about 10¹⁰, from about 10⁴ to about 10¹¹, from about 10⁵ to about 10⁶, from about 10⁵ to about 10⁷, from about 10⁵ to about 10⁸, from about 10⁵ to about 10⁹, from about 10⁵ to about 10¹⁰, from about 10⁵ to about 10¹¹, from about 10⁶ to about 10⁷, from about 10⁶ to about 10⁸, from about 10⁶ to about 10⁹, from about 10⁶ to about 10¹⁰, from about 10⁶ to about 10¹¹, from about 10⁷ to about 10⁸, from about 10⁷ to about 10⁹, from about 10⁷ to about 10¹⁰, from about 10⁷ to about 10¹¹, from about 10⁸ to about 10⁹, from about 10⁸ to about 10¹⁰, from about 10⁸ to about 10¹¹, from about 10⁹ to about 10¹⁰, from about 10⁹ to about 10¹¹, or from about 10¹⁰ to about 10¹¹ per mL. In some aspects, the liquid biofertilizer formulation has a CFU/mL of about 10², about 10³, about 10⁴, about 10⁵, about 10⁶, about 10⁷, about 10⁸, about 10⁹, about 10¹⁰ or about 10¹¹.

In one aspect, the biofertilizer formulation comprises the microorganism having an accumulation of a MISC greater than about 10%. In some aspects, the MISC is a PHA, a PolyP, or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In some aspects, the PHA is PHB.

In some aspects, the accumulation of MISC, such as PHB, is greater than about 10%, greater than about 12%, greater than about 15%, greater than about 20%, greater than about 25%, greater than about 30%, greater than about 35%, or greater than about 40%. In one aspect, the accumulation of MISC is greater than about 25%. In another aspect, the accumulation of MISC is about 30%. In another aspect, the accumulation of MISC is greater than about 40%.

In some aspects, the accumulation of MISC is from about 10% to about 12%, from about 10% to about 15%, from about 10% to about 20%, from about 10% to about 25%, from about 12% to about 15%, from about 12% to about 20%, from about 12% to about 25%, from about 12% to about 30%, from about 15% to about 20%, from about 15% to about 25%, from about 15% to about 30%, from about 15% to about 35%, from about 20% to about 25%, from about 20% to about 30%, from about 20% to about 35%, from about 20% to about 40%, from about 25% to about 30%, from about 25% to about 35%, from about 25% to about 40%, from about 30% to about 35%, from about 30% to about 40%, or from about 35% to about 40%.

In some aspects, the accumulation of MISC is about 10%, about 12%, about 15%, about 20%, about 25%, about 30%, about 35%, or about 40%.

In one aspect, the accumulation of MISC is about 25%. In one aspect, the accumulation of MISC is about 30%. In one aspect, the accumulation of MISC is about 40%.

In some aspects, the accumulation of MISC discussed herein is measured as the OD₆₀₀ of the culture of the microorganism after bleach digest of the microorganism over the OD₆₀₀ of the culture of the microorganism before bleach digest.

In some aspects, the biofertilizer comprises carriers selected from the following non-limiting types: liquid, soils, plant materials, or inert materials.

In some aspects, the soil carriers can be peat, coal, clays, humic acids, inorganic soils, unmodified soils, or compost amended soils, or a combination thereof. In some aspects, the inorganic soils can be clay soil, loess soil, clay minerals, kaolin, activated carbon fibers, or talc, or a combination thereof. In some aspects, the plant materials can be sawdust, lignin, soybean bran, wheat bran, oat bran, grass, brewery spent materials, winery spent materials, grape bagasse, cork compost, poultry manure, banana waste, or wastewater sludge, or a combination thereof. In some aspects, the inert materials can be polymers, silica, treated rock fragments, such as vermiculite and perlite. In some aspects, the polymers can be alginate, chitosan, corn starch, ethylcellulose, or modified starch, or a combination thereof. (Plant Soil, 2014, 378:1-33.)

Other suitable biofertilizer carrier materials can be, but are not limited to, peat-soil, histosol-soil, charcoal powder, rice husk ash, French chalk, or soapstone powder, or a combination thereof.

In some aspects, the biofertilizer can be formulated as an engineered soil.

In some aspects, the biofertilizer is stable for at least about 1 month, at least about 2 months, at least about 6 months, or at least about 12 months at room temperature or at about 4° C. In other aspects, the OD₆₀₀ of the biofertilizer changes within ±10% for at least about 1 month, at least about 2 months, at least about 6 months, or at least about 12 months at room temperature or at about 4° C. In other aspects, the amount of MISC changes within ±10% for at least about 1 month, at least about 2 months, at least about 6 months, or at least about 12 months at room temperature or at about 4° C.

### Methods/Uses

The disclosure further relates to a method of improving and/or maintaining crop or plant yield, yield quality, or plant aesthetics and/or improving soil health comprising administered the biofertilizer discussed herein to the crop or plant. In some aspects, the method can maintain crop yield after reducing conventional nitrogen fertilizer application. In some aspects, the crop yield can be, but is not limited to, yield quantity, quality or crop timings. In some aspects, the method can, but is not limited to, increase soil health through increased organic matter, lower soil nitrate and lower runoff potential or increased soil aggregation. In some aspects, plant aesthetics can be measured through chlorophyll or NDVI measurements.

In some aspects, the biofertilizer formulations can be applied directly to the field or used as a seed coating before sowing.

In some aspects, a liquid biofertilizer formulation comprises water, but can also comprise mineral or organic oils. Liquid formulations can be applied directly to the fields by mixing the portion of the liquid formulation with soil or by spraying the surface of the soil to be fertilized.

In some aspects, the crop or plant comprises one or more crops or plants from the following families: Asteraceae, Poaceae, Brassicaceae, Cucurbitaceae, Solanaceae, Rosaceae, Cannabaceae, Poaceae, Amaranthaceae, Amaryllidaceae, Polygonaceae, Liliaceae, Lamiaceae, Ericaceae or Fabaceae.

In other aspects, the crop or plant comprises one or more of the following crops or plants: a lettuce (Family: Asteraceae), a corn (Family: Poaceae), a cabbage (Family: Brassicaceae), a kale (Family: Brassicaceae), a cucumber (Family: Cucurbitaceae), a pepper (Family: Solanaceae), a tomato (Family: Solanaceae), a berry (Family: Rosaceae), a hemp (Family: Cannabaceae), a grass (Family: Poaceae), a turf, a beet (Family: Amaranthaceae), a squash (Family: Cucurbitaceae), an onion (Family: Amaryllidaceae), or a soybean (Family: Fabaceae).

In other aspects, the crop or plant comprises one or more of the following crops or plants: a baby leaf lettuce, a head lettuce, a sweet corn, a sweet pepper, a strawberry, or a raspberry.

In some aspects, disclosed herein is a method of improving soil health comprising applying any of the biofertilizers discussed herein to a crop or plant.

In some aspects, soil health is improved by reducing nitrogen leaching in the soil. In some aspects, the nitrogen leaching in the soil is reduced relative to soil that has not been administered the biofertilizer.

In some aspects, soil health is improved by increasing biological nitrogen fixation in the soil. In some aspects, the biological nitrogen fixation is increased relative to soil that has not been administered the biofertilizer.

### EXAMPLES

### Example 1. General Guidelines for Compound Nutrient Sources

A compound nutrient source comprises a phase separated material and complementary growth media formulation capable of releasing large quantities of growth supportive nutrients required for high cell density cultures. Use of such materials in culturing system can reduce or eliminate the need for multiple controlled additions of growth supportive nutrients. They do so by controlling the soluble concentration of a given nutrient through the solubility of that component dictated by the chemical equilibria of the material in the growth medium. The general characteristics, benefits, and theoretical framework to identify and design compound nutrient sources are described below.

Compound nutrient sources possess a finite solubility or miscibility that is lower than the solubility or miscibility of any one constituent component, typically as their nitrate (NO₃⁻), chloride (Cl⁻), hydroxyl (OH⁻), potassium (K⁺), or sodium (Na⁺) salts or acid forms (H+). In some aspects, the compound nutrient source can be a phase separated chemical compound of the general form:

AₐB_{b}C_{c}·nH₂O [symbol] aA+bB+cC+nH₂O,

where A, B, and C indicate constitutive components of phase separated compound nutrient AₐB_{b}C_{c} with stoichiometrical coefficients a, b, c, respectively. Such a compound has a solubility defined by the solubility product, Kₛₚ:

K_{sp,ABC}=[A]^{a}[B]^{b}[C]^{c}

where square brackets, [ ], denote the concentration or activity of the bracketed compound. The corresponding solubility of the individual constituents, e.g., A, as their nitrate, chloride, potassium, or sodium salt would satisfy the relationship:

$$A {a}^{0} Dd ⇌ {a}^{0} A + dD$$

$${K}_{sp , AD} = {{[ A ]}^{{a}^{0}} [ D ]}^{d}$$

$$\sqrt[{a}^{0}]{\frac{{K}_{sp , AD}}{{[ D ]}^{d}}} > \sqrt[a]{\frac{{K}_{sp , ABC}}{{{[ B ]}^{b} [ C ]}^{c}}}$$

where D=NO₃⁻, Cl⁻, OH⁻, Na⁺, K⁺, or H⁺, and d indicates the coefficient of the D salt. These compound nutrient sources can be further identified as any nutrient A, for which a saturated solution of AD, when combined with BD or CD, would produce an insoluble or immiscible phase separated compound (e.g., a precipitate).

These can be generalized for any compound nutrient source, X, with n stable soluble components Aᵢ with stoichiometric coefficient aᵢ as:

$$X ⇌ {\sum }_{i = 1}^{n} {a}_{i} {A}_{i}$$

$${K}_{sp , X} = {\prod }_{i = 1}^{n} {[ {A}_{i} ]}^{{a}_{i}}$$

For which any single component, A₁:

$${( {A}_{1} )}_{{a}_{1}^{0}} {D}_{d} ⇌ {a}_{I}^{0} ( {A}_{1} ) + dD$$

$${K}_{sp , AD} = {{[ {A}_{1} ]}^{{a}_{1}^{0}} [ D ]}^{d}$$

$$\sqrt[{a}_{1}^{0}]{\frac{{K}_{sp , AD}}{{[ D ]}^{d}}} > \sqrt[{a}_{1}]{\frac{{K}_{sp , X}}{{{\prod }_{i = 2}^{n} [ {A}_{i} ]}^{{a}_{i}}}}$$

These compound nutrient sources further bear the property of being a near infinite source of one or more constituent nutrients for microbial growth, while the balance of constituent components are removed from chemical equilibria by a chemical, biological reaction, phase separation, or other transformation. For an illustrative compound nutrient source, AₐB_{b}C_{c}, this would appear as:

a′Amicroorganism→Y

where a′ is the stoichiometric for the microbial conversion of A into product Y, where Y=biomass or other product of biological catalysis.

Residual components B and C could be removed from equilibria through precipitation:

b′B+c′C↔↓Z

Or through volatilization:

b′B+c′C↔↑Z

where b′ and c′ are the stoichiometric coefficients for the synthesis of phase separated component Z. In some aspects, compound Z may take the form of the precipitate B_{b}·C_{c′}. In some aspects, Z can be produced through reactive transformation of B and/or C into a precipitate, volatile gas, immiscible compound of derivative composition.

In the absence of this property, as consumption of A by microbial processes proceeds, accumulation of residual components B and C would lead to saturation, reducing the concentration of A to zero:

$$[ A ] = \sqrt[a]{\frac{{K}_{sp , ABC}}{{{[ B ]}^{b} [ C ]}^{c}}}$$

$$[ A ] = 0$$

The removal of B and C from the equilibrium governed by K_{sp,ABC} buffers against the saturation of either component, leading to an equilibrium concentration of A throughout the continuous dissolution of AₐB_{b}C_{c}. Furthermore, the removal of B and C from solution mitigates the accumulation of soluble species that would otherwise increase the ionic strength of the solution, which would be inhibitory to microbial growth due to osmotic stress.

An additional feature of a compound nutrient source is the ability to control the maximum concentration of any given constituent component. For certain nutrients, such as transition metals, quantities must be kept below an inhibitory threshold. With a compound nutrient source, the concentration of the supporting ions B and C can be altered by alternative sources, tailoring the concentration of A as per the relationship:

$${A}_{a} {B}_{b} {C}_{c} + {b}^{1} {B}_{{b}^{0}} {D}_{{d}^{′}} + {c}^{1} {C}_{{c}^{0}} {D}_{{d}^{″}} ⇌ aA + {b}^{0} {b}^{1} B + {c}^{0} {c}^{1} C + ( {b}^{1} {d}^{′} + {c}^{1} {d}^{″} ) D$$

$$[ A ] = \sqrt[a]{\frac{{K}_{sp , ABC}}{{{[ B ]}^{{b}^{0} {b}^{1}} [ C ]}^{{c}^{0} {c}^{1}}}}$$

where b¹ and c¹ are variable coefficients for salts BD and CD, respectively. As indicated in the equation above, the equilibrium concentration of A can be adjusted based on the inherent solubility dictated by K_{sp,ABC} as well as the addition of salts BD and CD, as well as bulk properties such as pH, headspace gas composition, agitation that affects the net concentration B and C. Additionally, any parameter that affects the value of K_{sp,ABC} itself, such as temperature, agitation rate, solution polarity, illumination levels may also be employed to tailor the equilibrium concentration of A.

### Example 2. Magnesium Ammonium Phosphates

For X. autotrophicus and most other microorganisms, significant quantities of nitrogen are required to achieve high cell densities. However, common nitrogen sources, such as NH₄⁺, exhibit inhibitory concentrations above 20 mM, a concentration only able to support an OD₆₀₀=2-4. As such, repeated manual or automated additions of NH₃ or NH₄⁺ are required, incurring additional hazards (i.e., from caustic NH₃ solutions) and hardware requirements for metering pumps, as well as potentially building up ionic concentrations to inhibitory levels due to unconsumed spectator ions (e.g., Cl from NH₄Cl).

In view of the above, a culturing system was developed that eliminates the need for multiple controlled additions of essential metabolites N, P, and Mg. Magnesium ammonium phosphates is a family of compounds used in the present culturing system as a sparingly soluble form of ammonium (NH₄⁺), magnesium (Mg²⁺) and phosphate (H₂PO₄⁻, HPO₄²⁻, PO₄³⁻) supportive of growth. The most common is the 1:1:1 stoichiometric salt known as struvite, MgNH₄PO₄, though magnesium ammonium phosphate compounds deviating from this stoichiometry and mixed phases possess similar properties with respect to solubility. The solubility limits of these compounds dictate a saturating equilibrium concentration of N, Mg and P below the toxicity level of X. autotrophicus, enabling the addition of excess quantities of magnesium ammonium phosphate in a single step. As the NH₄⁺ is consumed during the growth of X. autotrophicus, the magnesium ammonium phosphate dissolves and restores the equilibrium concentration of nitrogen (N). The governing chemical equations are:

Dissolution of Magnesium Ammonium Phosphate:

↓MgNH₄PO₄ [symbol] Mg²⁺+NH₄⁺+PO₄³⁻

K_{sp,struvite}=[Mg²⁺][NH₄⁺][PO₄^{3-]}

pH Dependent Phosphate Speciation:

H₃PO₄ [symbol] H⁺+H₂PO₄⁻

H₂PO₄⁻ [symbol] H⁺+HPO₄²⁻

HPO₄²⁻ [symbol] H⁺+PO₄³⁻

Precipitation of Magnesium Phosphates:

xMg²⁺+yH⁺+zPO₄³⁻ [symbol] ↓MgₓH_{y}(PO₄)_{z}

Growth of Microbial Biomass:

aMg²⁺+bNH₄⁺cPO₄³⁻+metabolites→biomass

Net Equations:

↓MgNH₄PO₄+yH⁺+metabolites→biomass

The equilibrium concentration of NH₄⁺ is dictated by the solubility product constant (K_{sp,struvite}) and the net concentration of Mg²⁺ and PO₄³⁻ in the solution. Furthermore, the concentration of PO₄³⁻ is highly pH-dependent. As such, the feature of magnesium ammonium phosphates are not only the material itself, but the complementary media and supplemental sources of Mg²⁺ (e.g. MgSO₄) and PO₄³⁻ (e.g. KH₂PO₄, K₂HPO₄). Finally, the precipitation reaction of Mg and P allows for the removal of magnesium phosphates from solution. Without this reaction, the unequal consumption of magnesium, ammonium and phosphate in the Growth of Microbial Biomass equation in which a≠b≠c would lead to saturating concentrations of Mg²⁺ and PO₄³⁻(→∞) and diminishing concentrations of NH₄⁺(→0). Considering the above, synthesis and addition of a compound source of nutrients (e.g. NH₄⁺) provides a steady state concentration of these nutrients, whose concentration is dictated by the concentrations of supporting species (e.g., Mg²⁺, PO₄³⁻) and bulk media characteristics (e.g. pH). Compared to other compound nutrient sources (e.g., polymer-coated slow-release nitrogen fertilizers), the delivery of NH₄⁺ is dictated not by the kinetics of transport (diffusion) from one phase to another, but rather the chemical equilibria of the compound itself.

For Xanthobacter autotrophicus, a single addition of NH₄⁺ (10 mM) below the inhibitory concentration (˜20 mM) yields a maximum cell density, measured by the optical density at 600 nm (OD₆₀₀), of 2-3. Addition of magnesium ammonium phosphate as the nitrogen source allows for a single dose addition to deliver NH₄⁺ at a steady state, non-inhibitory concentration to supply >20 mM NH₄⁺ providing the growth of X. autotrophicus to OD₆₀₀>4-6. As the starting magnesium ammonium phosphate and resulting magnesium phosphates, MgₓH_{y}(PO₄)_{z}, are solids and phase separated, their total mass does not affect chemical equilibria. As such, there is no theoretical limit to the amount of magnesium ammonium phosphate that can be added in a single dose, and therefore the resulting microbial biomass, except as dictated by practical and physical limitations such as the size of a give bioreactor, or the solid:liquid volume ratio. Cell densities of >40 OD₆₀₀ were achieved from a single addition of magnesium ammonium phosphate.

The magnesium ammonium phosphate can be from a variety of sources, including exogenously synthesized material obtained as magnesium ammonium phosphate hexahydrate (struvite), MgNH₄PO₄·6H₂O. The material can also be synthesized in situ by the simple mixing of a suitable magnesium source (e.g., Mg(OH)₂, MgCO₃) with ammonium phosphate (i.e. (NH₄)H₂PO₄). The in situ synthesis or precipitation additionally produces significantly smaller particle sizes, as they do not require milling or post processing, that is more favorable for the solid-liquid slurry of the magnesium ammonium phosphate medium.

### Example 3. Metal Carbonates

Metal carbonates of the general form (M)CO₃, where M is a divalent cationic metal (e.g., Ca²⁺, Mg²⁺, Fe²⁺, Ni²⁺, Co²⁺) represent a diverse class of materials that bear structural and chemical homology. Often, mixed metal solid solutions of multiple M species can be synthesized as stable compounds. The solubility of these ionic minerals is similarly dictated by the Kₛₚ, invoking the composition of the complementary medium, in particular the pH and headspace composition of the culture system. The thermodynamic and kinetic solubility of metal carbonates are in part dictated by the speciation of CO₃²⁻ and the corresponding carbonate series:

↑CO₂ [symbol] CO_{2(dissolved)}

CO_{2(dissolved)}+H₂O [symbol] H₂CO₃

H₂CO₃ [symbol] H⁺+HCO₃⁻

HCO₃⁻ [symbol] H⁺+CO₃²⁻

MCO₃ [symbol] M²⁺+CO₃²⁻

where M=Mg²⁺, Ca²⁺, Fe²⁺, Ni²⁺, Co²⁺, Mn²⁺ or other divalent cation.

Similar to magnesium ammonium phosphate, metal carbonates can function as a compound nutrient source. Solid solution compounds of the form:

Mₓ′M₁₋ₓ″CO₃ [symbol] xM′²⁺+(1−x)M″²⁺+CO₃^{2'}

where M′ and M″ represent two divalent metals that can function as compound nutrient sources as equilibrium controlled sources of growth essential trace metal and minerals. Solutions of more than two metals can also be formulated.

### Example 4. Media Formulation and Culturing Systems for Preferential Enhanced Accumulation of MISCs

While several microorganisms accumulate intracellular storage compounds, such as polyhydroxyalkanoates (PHAs), the accumulated PHAs' % is dependent on culture conditions and choice of carbon substrate. Typical microbial growth media and processes favor the replicative proliferation of cell numbers, balancing media components to favor the accumulation of high cell density rather than the accumulation of storage compounds. Furthermore, many bacteria contain competing pathways to accumulate extracellular storage compounds, such as exopolysaccharides, often referred to as slime. The slime presents a metabolic loss of carbon towards non-MISC, as well as acts as a surfactant to increase the generation of foam, complicating bioprocessing. The thickening property of the slime also increases the difficulty of processing and handling, in particular centrifugation and filtering steps. Other than genetic modifications of the microorganism to control the preferential accumulation of intracellular storage compounds, control over such a process can be provided by specific growth media formulations, nutrient programs and growth conditions in the cultivation process.

In this example, a media composition and growth system was developed to control the preferential accumulation of intracellular storage compounds in microorganisms of agricultural significance. In particular, preferential accumulation of PHB in X. autotrophicus over the production of extracellular slime can be achieved during growth on methanol through a limiting concentration of nitrogen and iron.

There are several conclusions from this example:
1. Growth on methanol and other autotrophic substrates (H₂/CO₂), as opposed to heterotrophic substrates (lactate, succinate) promotes low slime accumulation.
2. Non-exponential, linear growth promotes low slime accumulation, achieved through limiting any combination of dissolved O₂, carbon source, N, P, Fe, or other key nutrients.
3. A two-step growth process wherein an initial balance of C, N and P supports replicative growth at low % PHB accumulation. In a second step, at the exhaustion of N and/or P, continued feeding of C directs biomass accumulation towards MISC.

The C can be quantified using assays including but not limited to, total organic carbon (TOC) determination (www.epa.gov/sites/production/files/2015-12/documents/9060a.pdf), total carbon-inorganic carbon (TC-IC) (Journal of Experimental Marine Biology and Ecology 109.1 (1987): 15-23), or non-purgable organic carbon (NPOC) assay (www.umces.edu/sites/default/files/Dissolved%20Organic%20Carbon%20Method%2020 18-1_0.pdf). The N can be quantified using assays including but not limited to, total Kjeldahl method (www.epa.gov/sites/production/files/2015-08/documents/method_351-2_1993.pdf), ion selective electrode method (Aquaculture 450 (2016): 187-193), ion chromatography (Journal of Chromatography A 640.1-2 (1993): 161-165), combustion analysis or calorimetric determination (AOAC Official Method 972.43, Microchemical Determination of Carbon, Hydrogen, and Nitrogen, Automated Method, in Official Methods of Analysis of AOAC International, 18th edition, Revision 1, 2006. Chapter 12, pp. 5-6, AOAC International, Gaithersburg, MD., AOAC Official Method 990.03. Protein (Crude) in Animal Feed, Combustion Method, in Official Methods of Analysis of AOAC International, 18th Edition (2005). Revision 1, 2006, Chapter 4, pp. 30-31. AOAC International, Arlington, VA, and Journal of Agricultural and Food Chemistry 44.7 (1996): 1804-1807). The P can be quantified using assays including but not limited to, Briggs method (J. Biol. Chem. 53:13, J. Biol. Chem. 59:252-264), molybdenum blue method (Analytical Chimica Acta, 27, 31-36), malachite green assay (Analytical biochemistry 161.1 (1987): 45-48) or orthophosphate digestion (Limnology and Oceanography: Methods 15.4 (2017): 372-380). All references cited herein are incorporated by reference in entirety.

With the methods described in the example, the growth of Xanthobacter autotrophicus to an intracellular % PHB concentration of >25% was demonstrated, compared to a naturally occurring concentration generally in the range of <10%. Several aspects to this performance were identified. These aspects can work independently or as a combination thereof:
1. Utilization of methanol in a fed batch process to achieve non-exponential, linear growth;
2. Compound nutrient source, such as struvite, as a concentration-limiting source of N, P and Mg to further reinforce linear growth; or
3. Based on experimental, empirical determinations of N:biomass stoichiometry, additions of methanol beyond the limiting stoichiometry dictated by struvite promote the preferential accumulation of PHB while maintaining low slime production.

### Methods

Microbial cultures of Xanthobacter autotrophicus were routinely maintained at −80° C. in 5% DMSO. Before inoculation, microbial cultures were revived by streaking cryopreserved stocks onto Rich Broth plates (16 g/L BD Difco Nutrient Broth, 15 g/L agar) and incubating at 30° C. Single colonies and multiple colonies were selected from the plates after 2-3 days of growth and transferred into shake flasks containing liquid undefined media, Rich Broth (16 g/L BD Difco Nutrient Broth) and further incubated with vigorous (>200 rpm) shaking at 30° C.

Cultures were typically expanded through a seed train from a pure Rich Broth culture (25 mL) by serial dilutions with 2-3× dilutions into sterile Defined Medium (DM_mod 4) as defined below:

TABLE 1
DM mod4 Medium Composition

| Component | | Quantity | |
|---|---|---|---|
| 1 | K₂HPO₄ | 1.6 | g/L |
| 2 | KH₂PO₄ | 1.5 | g/L |
| 3 | MgSO₄•7H₂O | 0.1 | g/L |
| 4 | CaSO₄•2H₂O | 0.01 | g/L |
| 5 | (NH₄)₂SO₄ | 0.66 | g/L |
| 6 | Iron stock solution* | 0.25 | mL/L |
| 7 | Trace metal mix** | 1 | mL/L |

| | | |
|---|---|---|
| 8 | Methanol | 4 mL/L for Δ2 OD₆₀₀ |
| 9 | Struvite (MgNH₄PO₄•6H₂O)^{†} | 10 g/L for Δ20 OD₆₀₀ |
| | or | or |
| | (NH₄)HCO₃^{‡} | 0.76 g/L for Δ2 OD₆₀₀ |

Note:
components 5-7 are sterilized by filtration.

TABLE 2
Iron Stock Solution Composition
*Iron stock solution

| Component | Quantity | |
|---|---|---|
| Ferric citrate | 50 | g/L |
| (NH₄)HCO₃ | 48.5 | g/L |

TABLE 3
Trace Metal Mix Composition
**Trace metal mix

| Component | Quantity | |
|---|---|---|
| H₃BO₃ | 2.8 | g/L |
| MnSO₄•H₂O | 1.6 | g/L |
| Na₂MoO₂•2H₂O | 0.75 | g/L |
| ZnSO₄•7H₂O | 0.24 | g/L |
| Cu(NO₃)₂•2.5H₂O | 0.04 | g/L |
| NiSO₄•6H₂O | 0.13 | g/L |

TABLE 4
Struvite Alternative Preparation
†Struvite (alternative preparation)

| Component | Quantity |
|---|---|
| (NH₄)H₂PO₄ | 0.468 g/g struvite |
| Mg(OH)₂ | 0.238 g/g struvite |

†Struvite can be sourced as-prepared, or synthesized from the precursor components.
To synthesize struvite, a sterile solution of (NH₄)H₂PO₄ was combined with sterilized Mg(OH)₂ or a suitable stoichiometric magnesium carbonate substitute through simple mixing in deionized water. Struvite synthesis may also be prepared in situ by addition of precursors discretely in the DM_mod4, or directly in the culture vessel/bioreactor.
‡As an alternative N source, a single addition of an arbitrary amount of struvite may be substituted by sequential additions of (NH₄)HCO₃. The additions of (NH₄)HCO₃ should be subdivided so as to not exceed a concentration of 10 mM in solution.

At each seed train stage, the culture was grown to an OD₆₀₀ of >2 before dilution/volume expansion. Cultures were expanded up to a volume of 1.5 L, whereupon sequential additions of 4-8 mL/L of neat methanol were made to the culture to grow an additional 2-4 OD₆₀₀ per addition, proportionally. The 1.5 L of culture was so grown up to an OD₆₀₀>20, after which it was suitable as an inoculum at a dilution rate of 10×. Subsequent growth in DM_mod 4 with serial additions of methanol up to an OD₆₀₀>25 were performed in bioreactors of 10 L, 100 L, 1000 L of various designs, including impeller-mixed and air-lift style aerobic bioreactors.

At the final expansion volume (e.g. 100 L), intracellular PHB was accumulated through continued additions of the carbon substrate, e.g. methanol. Once the OD₆₀₀ exceeded the stoichiometric limit of N as dictated by the amount of struvite (e.g. 10 g/L for a ΔOD₆₀₀=20), continued methanol additions were made, accompanied by monitoring of intracellular PHB accumulation through a bleach digestion assay. In brief:
1. Aliquots of microbial culture were washed 2× in a 100 mM sodium citrate buffer through centrifugation and resuspension of the pellet in order to remove any insoluble precipitates.
2. Total optical density (OD_{600,tot}) was measured by appropriate dilution in a 100 mM sodium citrate buffer.
3. The undiluted, washed sample was mixed at a ratio of 1:4 (aliquot:bleach) with a commercial bleach solution (sodium hypochlorite) and left agitating on an orbital shaker for 2 hrs to digest non-PHB cellular material.
4. The optical density of the bleach digested sample (OD_{600,digest}) was measured and compared against the total to determine the % intracellular PHB according to the equation:

$$\% PHB = \frac{0 {D}_{6 \; 00 , digest}}{0 {D}_{6 \; 00 , tot}} \times 100 \%$$

Methanol additions were continued until a % PHB of >25% was obtained.

### Results

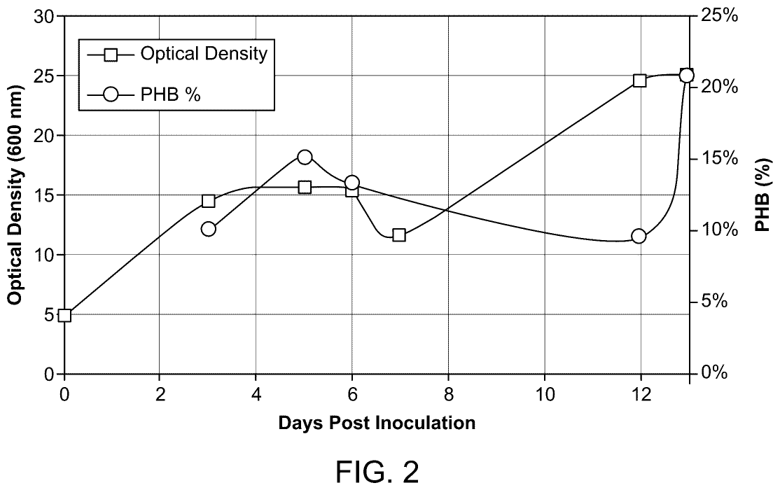

FIG. 2 shows a plot of the OD₆₀₀ and PHB accumulation throughout the course of a developmental batch. In the plot, there are two linear growth phases, Days 0-3 and Days 7-12, followed by two stationary phases, Days 3-6 and Days 12-13 respectively. As indicated, the accumulation of PHB lagged behind the growth phases and the bulk of PHB was accumulated during the stationary phases. The % PHB decreased during the growth phases. The decrease in PHB during growth was either caused by consumption of PHB to fuel growth or that growth was occurring faster than PHB was accumulating and being distributed between a larger number of microbes. The PHB accumulation can be induced by limiting one of growth nutrients (C, N, O₂, CO₂, Fe, etc.). As shown in the plot, PHB accumulation occurred at Day 12 when the growth ceased from exhaustion of N. Table 5 below shows examples of end-point measurements for several batches of biofertilizers. The OD₆₀₀ values range from about 25 to about 36 and the PHB % range from about 25% to about 45%.

TABLE 5
End-point Measurements of OD₆₀₀
and PHB % from Several Batches

| Reactor | OD₆₀₀ | PHB |
|---|---|---|
| Bulbasaur_20200312_10L | 25.6 | 25.38% |
| Charmander_20200407 | 27.2 | 33.01% |
| Charmander_20200421 | 35.5 | 43.19% |
| Charmeleon_20200526 | 32.5 | 33.92% |
| Ivy/Char Drum 20200422 | 26.2 | 27.02% |

### Example 5. Biofertilizer Transport, Storage and Stability

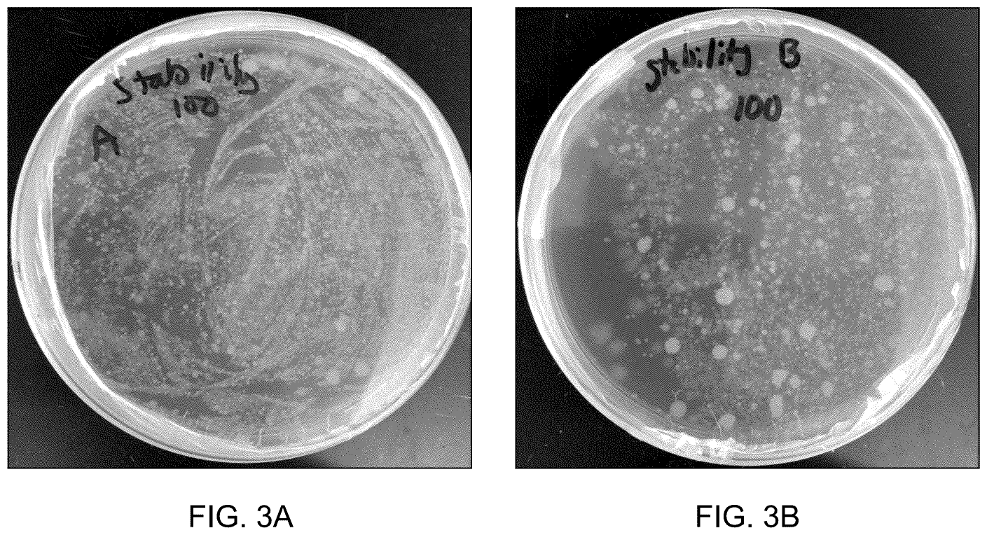

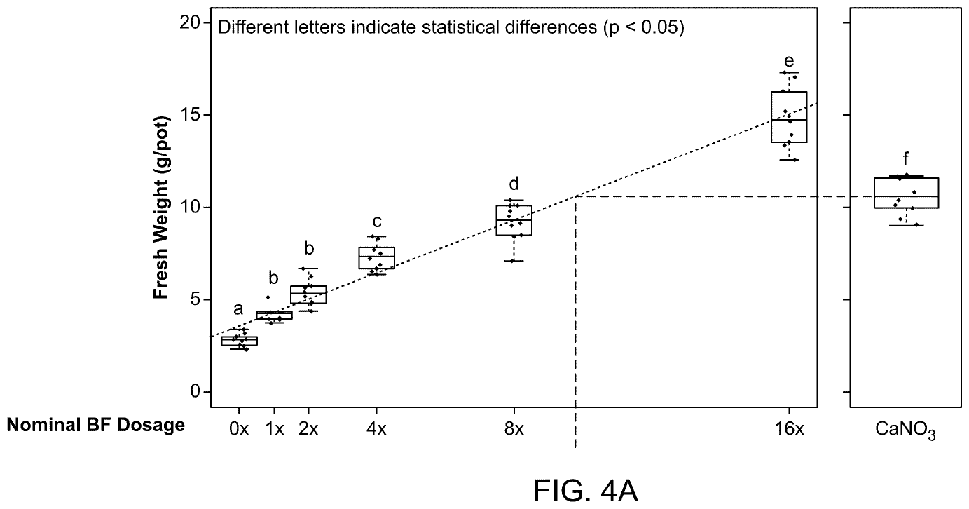

There is no noticeable change in product efficacy for up to about 1 week, or about 1 month or longer, stored in refrigeration. A test was conducted with one sample shipped to another state, held at refrigeration, returned without refrigeration in 2 days. The control sample was held at the laboratory at 4° C. during the same period of time. After 1 week of storage and transport, the sample was measured for OD₆₀₀, PHB content and CFU and showed about 10% loss in OD₆₀₀ but about 10% increase in % PHB, while the control sample lost about 20% in OD₆₀₀ and had about 15% increase in % PHB. FIG. 3A and FIG. 3B show the resulting colony forming units that correlate well with the OD₆₀₀ values from the viability assays for the control and the sample, respectively.

### Example 6. Greenhouse Trial-Dosing Calibration

The biofertilizers were tested in various greenhouse experiments and trials across diverse plant and crop types, including but not limited to, head lettuce, baby leaf lettuce, sweet corn, tomatoes, peppers, cucumbers, cabbage, strawberries, and orchardgrass.

The greenhouse experiment with baby leaf lettuce established the initial benchmarking and calibrated dosing of biofertilizer products and processes. The biofertilizer products were prepared in shake-flasks on Rich Broth medium to an initial OD₆₀₀ of about 1.6 and a % PHB of about 3-5 wt %. The biofertilizer used in the experiment had a low % PHB. The biofertilizer medium contained trace residual nitrogen from NO₃⁻ and amino acids, and residual carbon from sugars and organic acids. The positive control fertilizers were calcium nitrate or urea, applied at the rate of about 20 lb N/acre. The soil was the PRO-MIX® potting media (peat moss) with mycorrhizal inoculant.

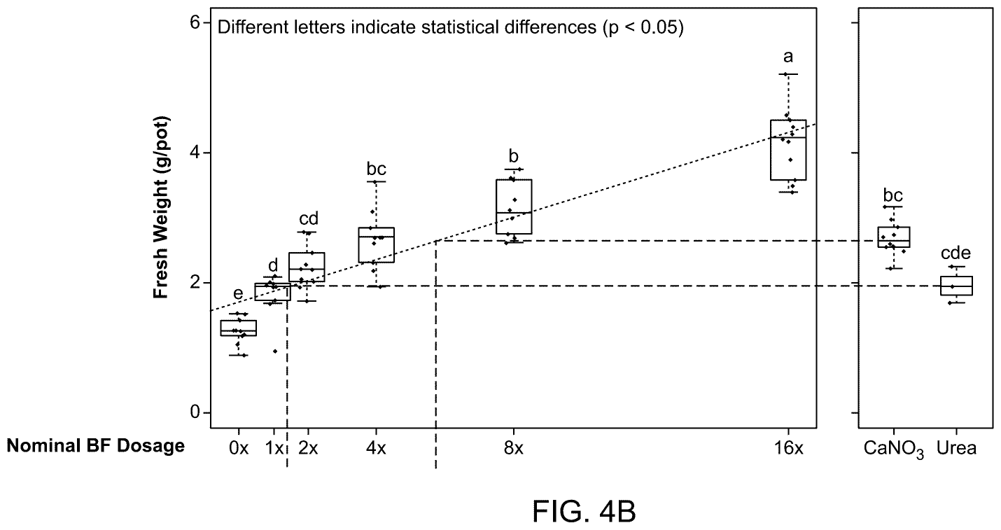

The dosings of biofertilizer were 0, 1, 2, 4, 8, and 16 times of the biofertilizer products, which correspond to the range of about 20-40 lbs of nitrogen. Calcium nitrate or urea were applied to conventional (positive control) plants at the rate of about 20 lb nitrogen/acre. The plants were harvested and evaluated three times every two weeks. The first harvest was after 3 weeks with no fertilizer applied to pull out the background nitrogen in the soil. The second harvest was 2 weeks after the conventional or biofertilizer application which was immediately carried out after the first harvest. The third harvest was after another period of no fertilizer application to examine the carry-over effect from previous fertilizer applications. As shown in FIG. 4A, a linear growth response with increasing dosages of biofertilizers in the fresh weights was observed at the recommended fertilization range of 20-40 lb N. At 16 times dosing, the biofertilizer outperformed conventional fertilizer. As shown in FIG. 4B, at 8 times dosing or higher, the carry-over fertilization outperformed calcium nitrate fertilization and at 2 times dosing or higher, the carry-over fertilization resulted in larger fresher weights than urea fertilization. Accordingly, the dosing was set at 0.002 lb N/OD₆₀₀. L. Based on the current trial result, for a biofertilizer product at an OD₆₀₀ of about 45, the crop product had a N:P:K ratio of 0.1:1.1:0.6 with a delivered N:P:K ratio of 4.1:1.1:0.6 from a fertilizer.

### Example 7. Field Trials—Nitrogen Stimulation

Field trials on romaine lettuce and sweet corn revealed key advantages of the biofertilizer products and performance.

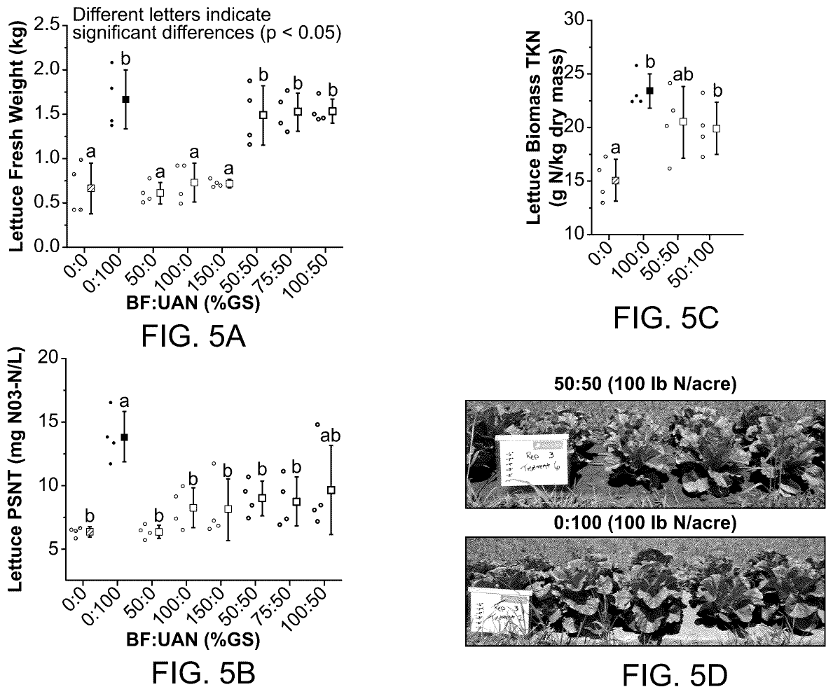

The field trials were designed to compare biofertilizer of the present disclosure, a synthetic fertilizer, and the co-applications thereof. The synthetic fertilizer is urea-ammonium-nitrate (UAN). The applications of ratios of biofertilizer:UAN ranges from 0:0, 0:100, 50:0, 100:0, 150:0, 50:50, 75:50, and 100:50. The ratios of 150:0 and 100:50 indicate 50% more nitrogen fertilization rate. The ratio of 75:50 indicates 25% more nitrogen fertilization rate. Pre-sidedress nitrate test (PSNT) of the 0:0 indicates the sample was not nitrogen saturated and the yield is limited by other nutrients (e.g., P or K). Low PSNT values indicate low runoff potential of the soil. As shown in FIG. 5A, co-applications of biofertilizer and UAN all show identical yields to the UAN control. Increasing dosing of biofertilizers narrowed the variance in the fresh weights of lettuce plants among the plot replicates (e.g., comparing among the ratios of 50:0, 100:0, and 150:0, or among the ratios of 50:50, 75:50, and 100:50). The differences between biofertilizer only and biofertilizer and UAN applications indicate the stimulatory need for nitrogen, which might be highly dependent on the type of soil or plant. The low PSNT values of biofertilizer applications indicate low runoff potential and the low risk of over-fertilization using biofertilizers as indicated in FIG. 5B. The lower capital letters above the data bars indicate significant differences between fertilization using Tukey's HSD test (p<0.05). The comparable biomass TKN among the ratios of 0:100, 50:50, and 100:50 biofertilizer:UAN indicate that co-applications of biofertilizer and synthetic fertilizers maintain similar biomass TKN as compared to the efficacy of synthetic fertilizer applications.

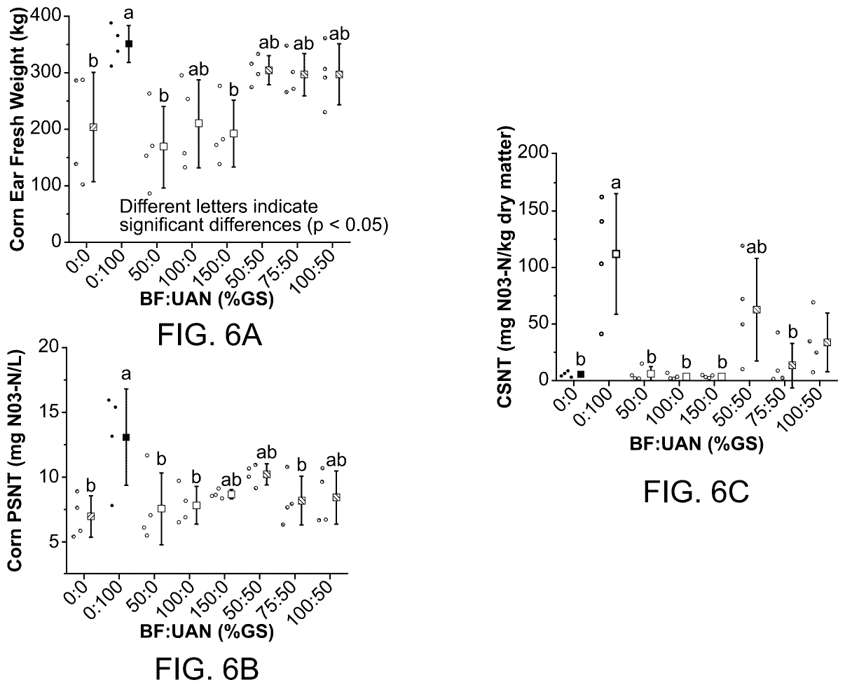

A similar trend was observed with sweet corn plants. As shown in FIG. 6A, co-applications of biofertilizer and UAN all show identical yields to the UAN control. Increasing dosing of biofertilizers narrows the variance in the corn ear fresh weights of sweet corn plants among the plot replicates (e.g., comparing among the ratios of 50:0, 100:0, and 150:0, or among the ratios of 50:50, 75:50, and 100:50). The differences between biofertilizer only and biofertilizer and UAN applications indicate the stimulator need for nitrogen, which might be highly dependent on the type of soil or plant. The ratios of 150:0 and 100:50 indicate 50% more nitrogen fertilization rate. The ratio of 75:50 indicates 25% more nitrogen fertilization rate. Pre-sidedress nitrate test (PSNT) of the 0:0 indicates the sample was not nitrogen saturated and the yield is limited by other nutrients (e.g., P or K). Low PSNT values indicate low runoff potential of the soil. The lower capital letters above the data bars indicate significant differences between fertilization using Tukey's HSD test (p<0.05). Co-applications of biofertilizer and UAN all resulted in comparable quality metrics (e.g., TKN, corn ear weight, quality, or vigor). The low PSNT values of biofertilizer applications indicate low runoff potential and the low risk of over-fertilization using biofertilizers. The corn-stalk nitrate test (CSNT) values indicate plants with co-applications of biofertilizers and UAN contain less nitrate in the biomass.

### Example 8. Greenhouse Trials—Nitrogen Stimulation

The nitrogen stimulation trend was tested and seen across a variety of crops and plants. Paralleled performances were observed across a wide variety of vegetable and fruiting crops and plants, including but not limited to, peppers, tomatoes, cucumbers, cabbage, kale, strawberries and head lettuce. The trials aimed to optimize the formulation, timing, and application methods.

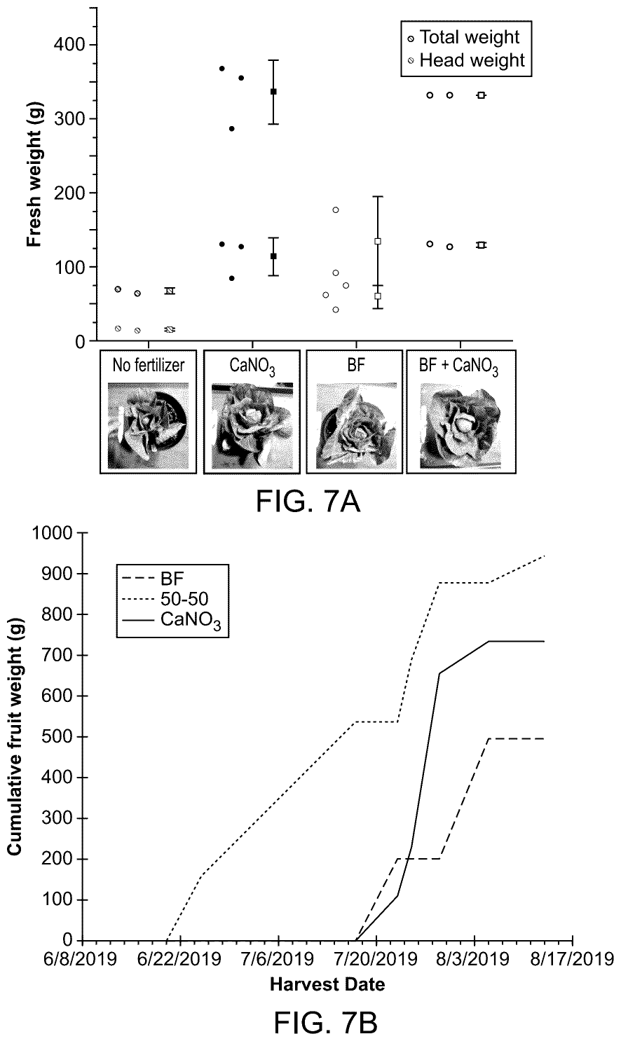

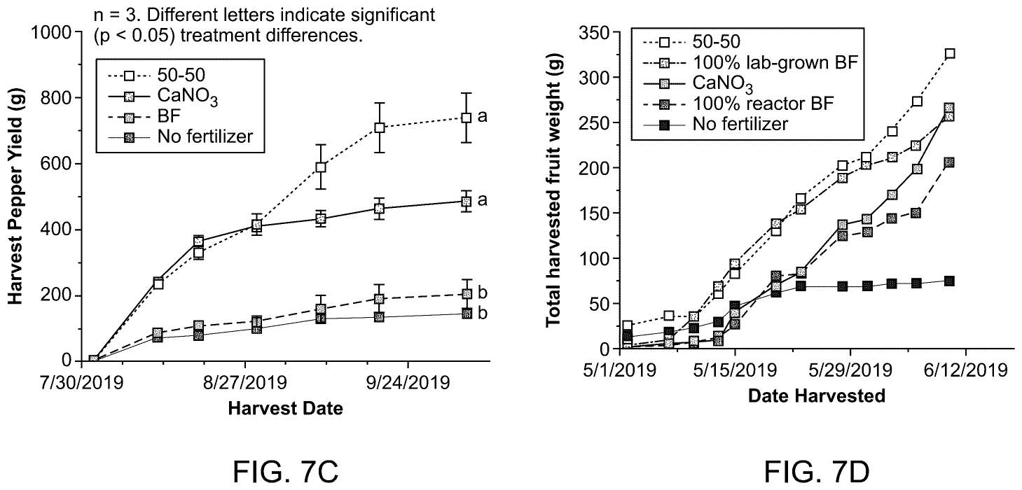

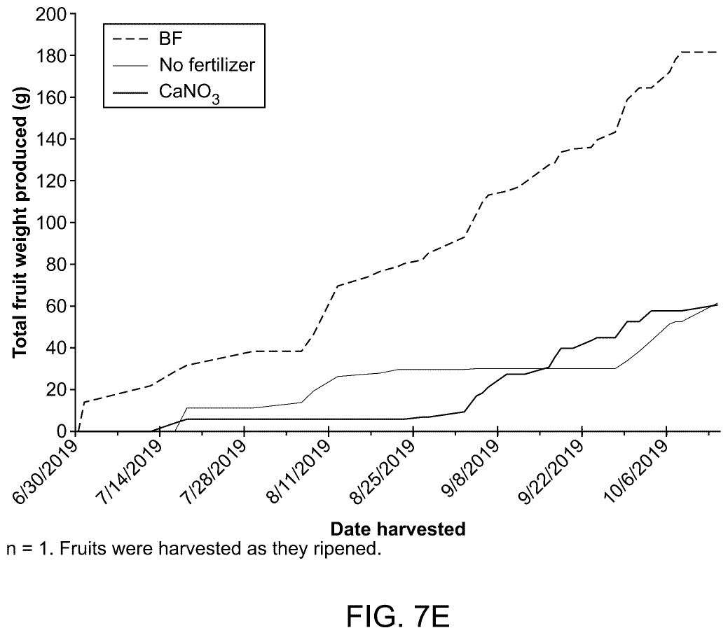

The fertilizers were applied every 2 weeks. As shown in FIG. 7A, the cabbage fresh weights (g) were measured across 4 applications: no fertilizer, calcium nitrate, biofertilizer, and co-application of the biofertilizer of the present disclosure and calcium nitrate. The total fertilization rate was at 180 lb nitrogen/acre for all applications. Fertilization with calcium nitrate only and with co-application of biofertilizer and calcium nitrate resulted in similar total weight and head weight. The cucumber cumulative fruit weights (g) were measured across applications of the biofertilizer only, 50:50 biofertilizer:calcium nitrate, and calcium nitrate only in FIG. 7B. The total fertilization rate was at 116 lb nitrogen/acre for all applications. The results show that 50:50 co-application of biofertilizer:calcium nitrate resulted in largest cumulative fruit weight, followed by calcium nitrate application, and biofertilizer only application. FIG. 7C shows the harvested pepper yields (g) from sweet pepper plants across applications of 50:50 biofertilizer:calcium nitrate, calcium nitrate only, biofertilizer only, and no fertilizer. The total fertilization rate was at 275 lb nitrogen/acre for all applications. There were three replicates for each application. The lower capital letters at the right end of the data lines indicate significant differences between fertilization using Tukey's HSD test (p<0.05). The results show that 50:50 co-application of biofertilizer:calcium nitrate or calcium nitrate only application resulted in larger pepper yield than biofertilizer only, and no fertilizer. FIG. 7D shows the total harvested fruit weights (g) from cherry tomato plants across applications of 50:50 biofertilizer:calcium nitrate, biofertilizer only (lab grown), calcium nitrate only, biofertilizer only (reactor grown), and no fertilizer. The lab-grown biofertilizer was prepared with residual nitrate. The total fertilization rate was at 120 lb nitrogen/acre for all applications. The results show that 50:50 co-application of biofertilizer:calcium nitrate resulted in largest total harvested fruit weight, followed by the other applications, last by no fertilizer application.

Further, the soil type effects and the biofertilizer application were tested with perennials. As shown in FIG. 7E, the total fruit weights produced (g) from strawberry plants were measured across applications of biofertilizer only, no fertilizer, and calcium nitrate only. The total fertilization rate was at 200 lb nitrogen/acre for all applications. Specifically, the fertilization rate was 18 lb nitrogen per every 2 weeks. Application of biofertilizer only outperformed the other applications in resulting less vegetative growth, earlier and more vigorous flowering, and the largest total fruit weight produced.

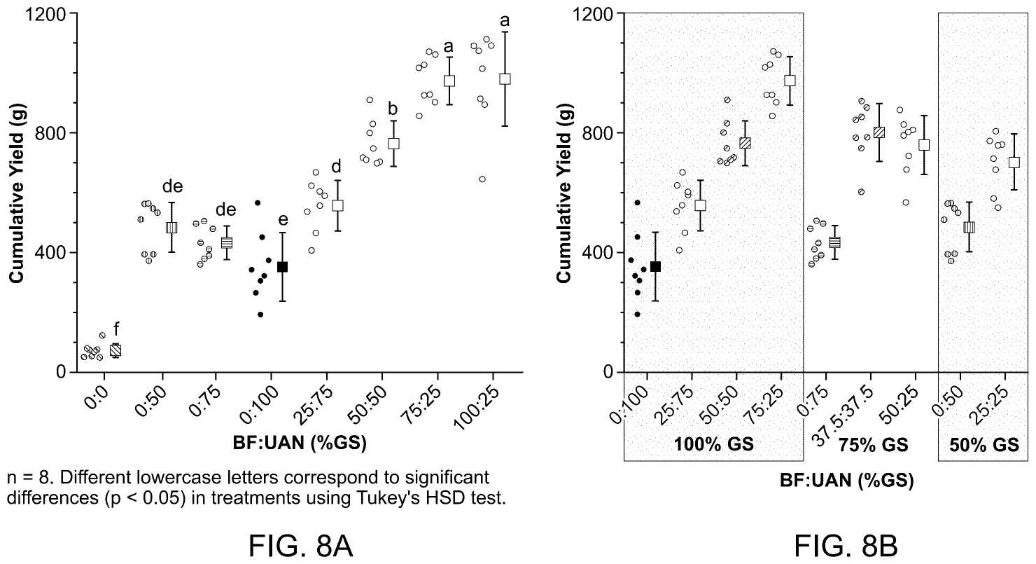

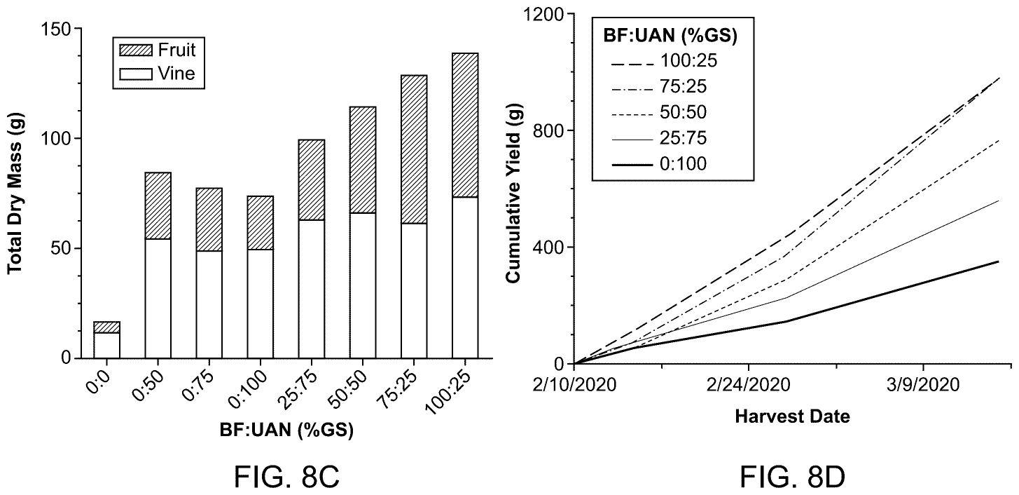

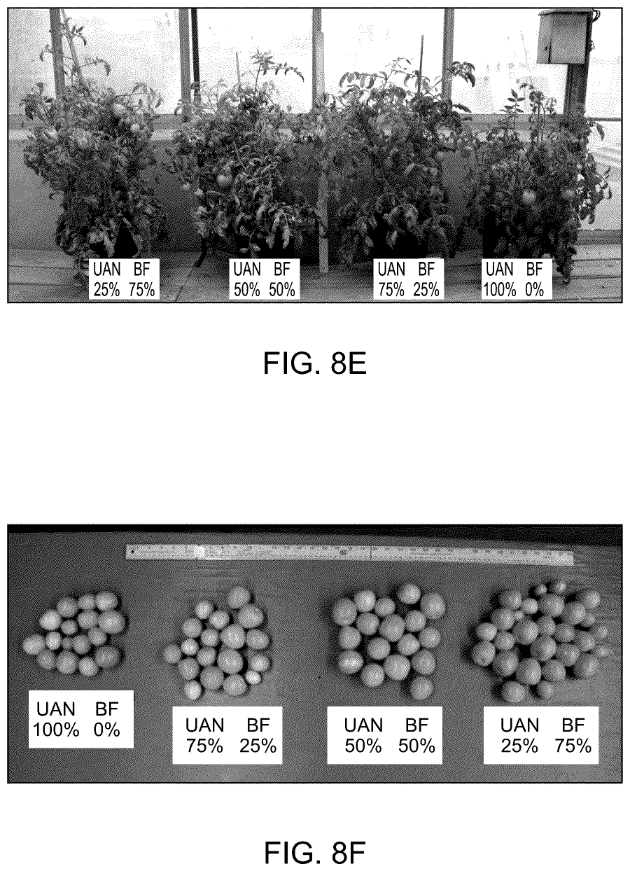

Another example is a greenhouse trial of biofertilizer applications on tomato plants. The fertilizer applications were conducted every 2 weeks. The 100% Grower's Standard (GS) was 150 lb nitrogen/acre. The ratios of biofertilizer:UAN (% GS) tested were 0:0, 0:50, 0:75, 0:100, 25:75, 50:50, 75:25 and 100:25. Each ratio had 8 replicates. As in FIG. 8A, the results show that the plants achieved 150% yield increase with biofertilizer replacement. UAN controls (0:50, 0:75, and 0:100) decreased yield as the % GS increased indicates over fertilization. Excess dosing of biofertilizer replacement did not decrease yield. The results also indicate larger replacement rates linearly increase yield and thus 75% biofertilizer replacement rate was shown efficient. FIG. 8B shows blend applications of biofertilizer and UAN had higher yield than UAN only application. At 100% GS, the blends linearly increased the yield but the same trend was not observed with 75% GS, among the ratios of biofertilizer:UAN 0:75, 37.5:37.5, and 50:25. The vegetative (vine) biomass was consistent across all ratios, as in FIG. 8C, even with 75% GS reduction of UAN. Fruit biomass increased with the increasing biofertilizer ratios. The cumulative yields (g) from tomato plants applied with different ratios of biofertilizer:UAN were measured at different harvest dates. The results indicate that typical yield was reached up to three weeks earlier in the biofertilizer blend applications. The typical yield is defined as the final yield produced by grower standard practices (0:100).

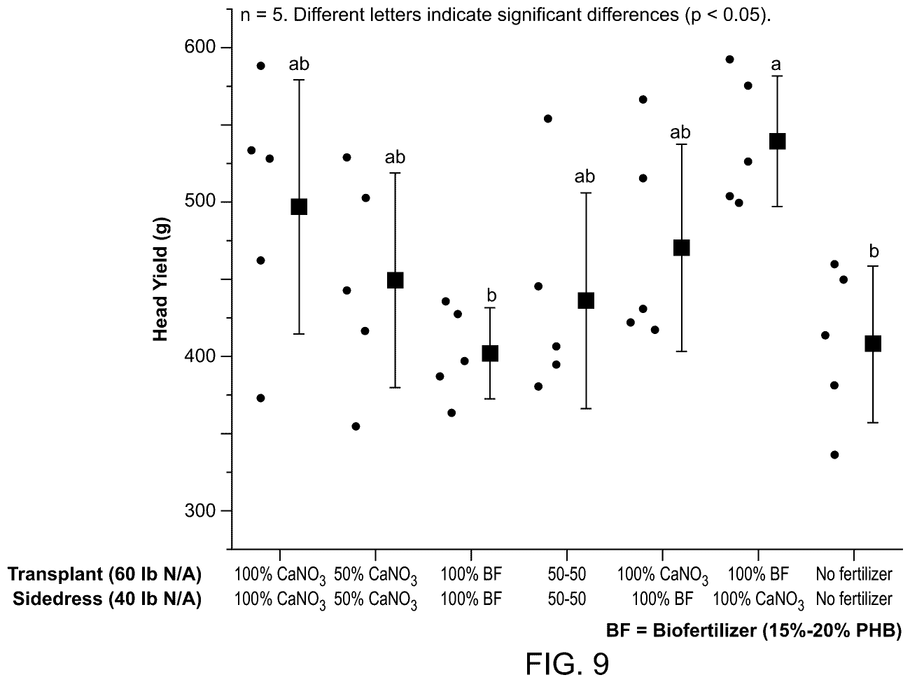

The timing of nitrogen priming was tested with head lettuce. Fertilizer applications at the time of transplantation or at the time of sidedressing were compared. The head lettuce plants were transplanted into compost amended soil to simulate organic soils. Biofertilizers (enhanced with 15-20% intracellular PHB) and calcium nitrate controls were applied sequentially at the time of transplantation (at the rate of 60 lb nitrogen/acre) and by sidedressing (at the rate of 40 lb nitrogen/acre), or both, as indicated in FIG. 9A. The applications timing and methods include: 100% calcium nitrate at transplantation followed by the same at sidedress; 50% calcium nitrate at transplantation followed by the same at sidedress; 100% enhanced biofertilizer at transplantation followed by the same at sidedress; 50:50 enhanced biofertilizer:UAN at transplantation followed by the same at sidedress; 100% calcium nitrate at transplantation followed by 100% enhanced biofertilizer at sidedress; 100% enhanced biofertilizer at transplantation followed by 100% calcium nitrate at sidedress; or no fertilizer at either timing. The head yields were compared across different application timing and methods. The application of PHB-enhanced biofertilizer at the transplantation followed by calcium nitrate sidedress produced the largest head yield (g). Sidedress nitrate was crucial to high yield. The results also indicate enhanced biofertilizer works best as a slow release fertilizer in container production and with precision application to the roots zoon, and is best timed for slow-uptake portion of plant growth cycle, i.e., at the transplantation.

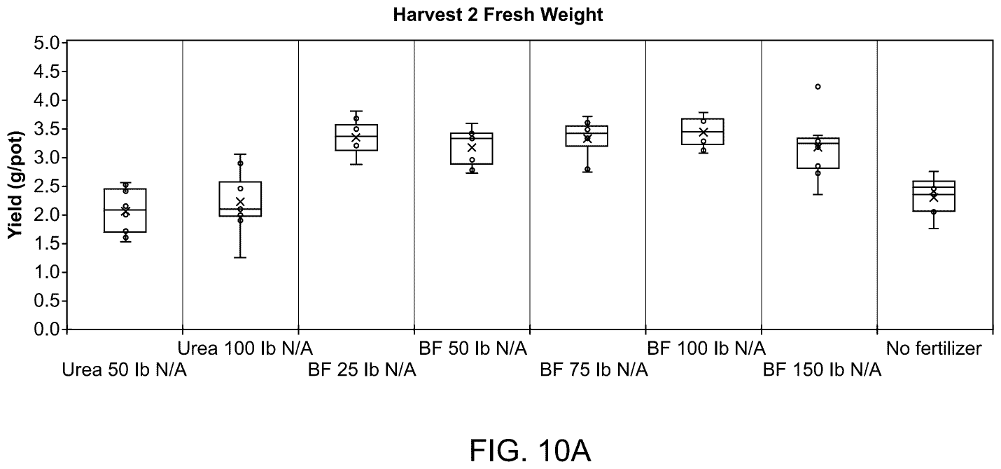

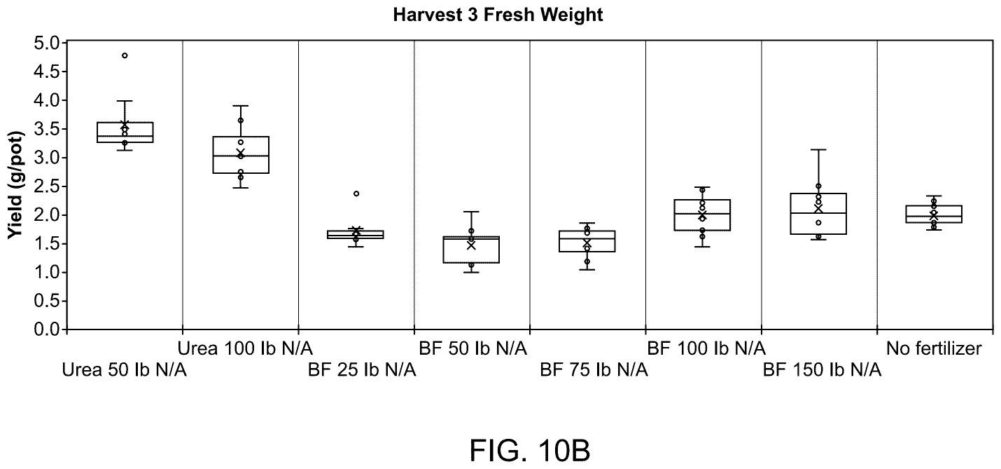

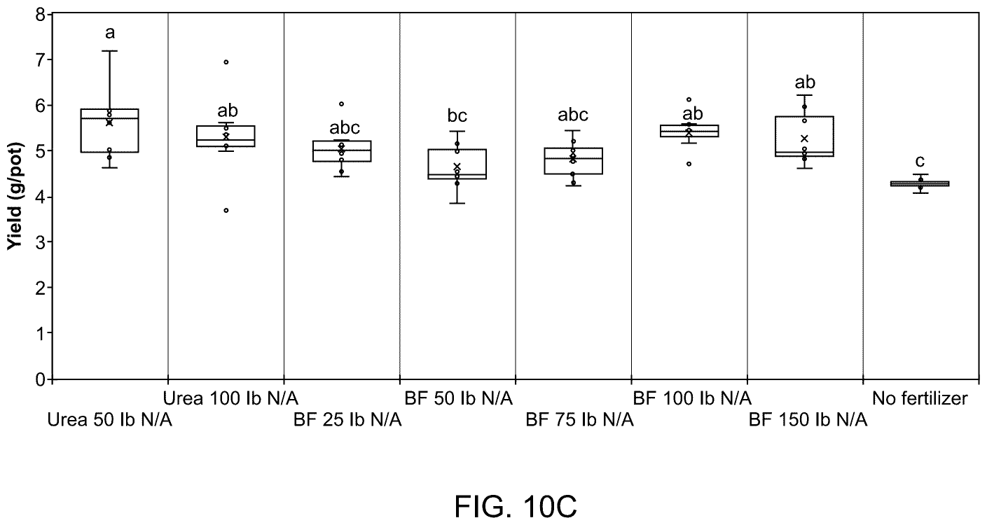

Greenhouse trials with orchardgrass provide an example of the biofertilizer efficacy at high dosages (<150 lb N/acre) and on a grass-type crop. Urea was used as control. The fertilizers were applied in 50 mL solution after the establishment period of the orchardgrass. The fertilizer applications include 50 or 100 lb nitrogen/acre urea, 25, 50, 75, 100, or 150 lb nitrogen/acre biofertilizer, and no fertilizer. There were 8 replicates for each application. The grass was harvested every 2 weeks. The fresh yields from the combined results of the second and third harvests, as shown in FIG. 10C, indicate that biofertilizer dosage as low as 25 lb N/acre achieves similar performance as synthetic fertilizer urea at 50 or 100 lb N/acre.

### Example 9. Greenhouse Trials—Enhanced PHB Accumulation

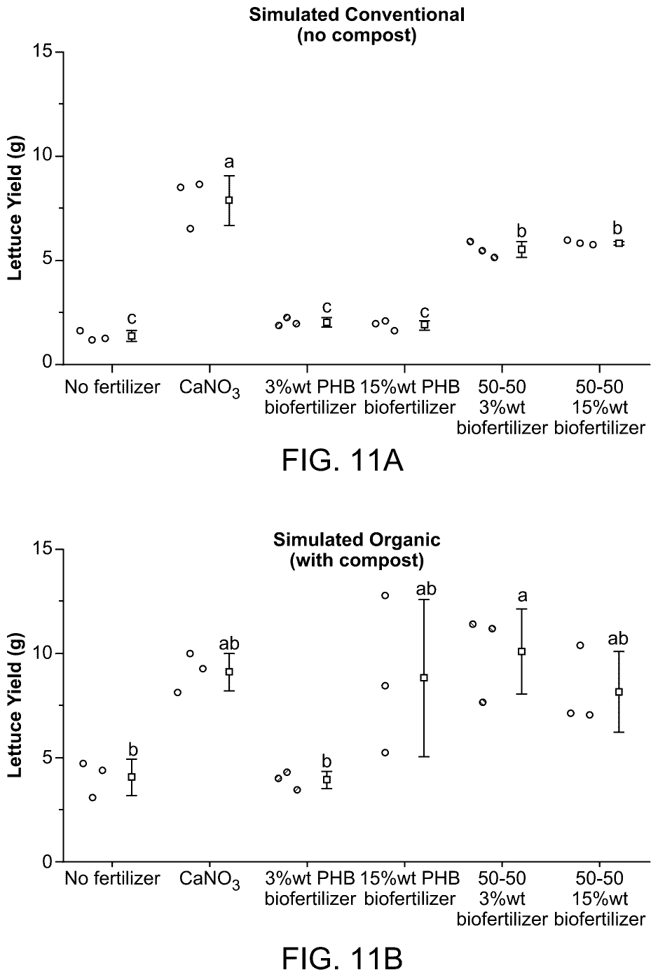

A greenhouse trial experiment with baby leaf lettuce was conducted to compare the effects of soil quality and PHB content on the biofertilizer's efficacy. The baby leaf lettuces were transplanted into either soil amended with compost or soil not amended to simulate organic or conventional soil for comparison. There were two harvests every 2 weeks for each plant. The first harvest was after a period of no fertilizer for background nitrogen. The second harvest was after a period with fertilization and the yield, soil nutrient, soil microbial population were measured. The fertilization applications include no fertilizer, calcium nitrate only, biofertilizer enhanced with 3 wt. % intracellular PHB only, biofertilizer enhanced with 15 wt. % intracellular PHB only, 50:50 3 wt. % intracellular PHB biofertilizer:calcium nitrate, 50:50 15 wt. % intracellular PHB biofertilizer:calcium nitrate. As shown in FIG. 11A and FIG. 11B, in the compost amended soil, the enhanced biofertilizers including 15 wt. % intracellular PHB biofertilizer only, 50:50 3 wt. % intracellular PHB biofertilizer:calcium nitrate, and 50:50 15 wt. % intracellular PHB biofertilizer:calcium nitrate performed as well as calcium nitrate. 15 wt. % intracellular PHB biofertilizer showed dramatic benefit over 3 wt. % intracellular PHB biofertilizer. Applied onto the not amended soil, the enhanced biofertilizers resulted in lower yield as compared to calcium nitrate.

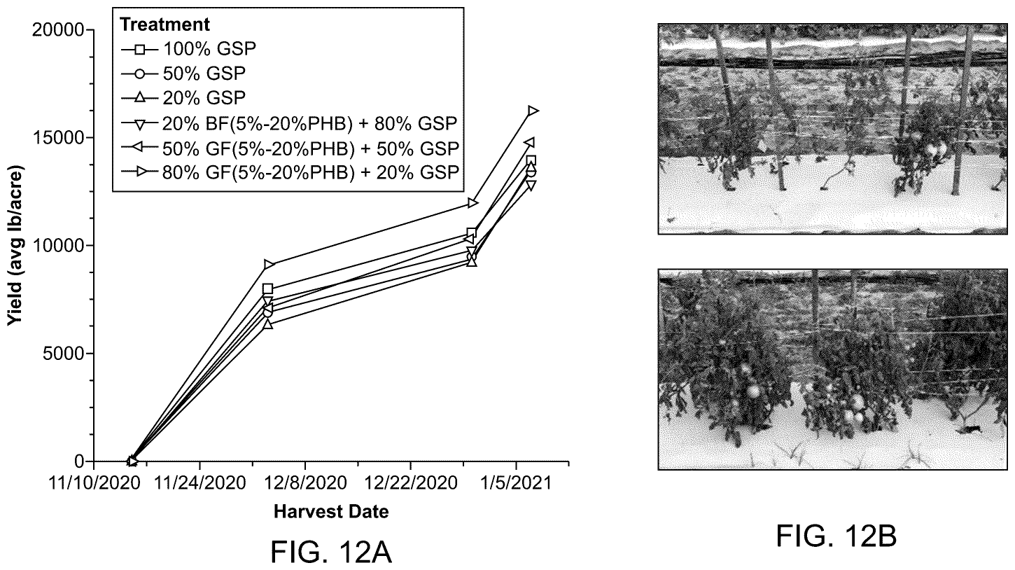

Further experiment trials on the dosing and blend of the biofertilizer with the synthetic fertilizers across a variety of crops and plants are in progress. Over seven external field and greenhouse studies, biofertilizer successfully achieved replacement for conventional nitrogen fertilizers between 20-100%. Crops included head lettuce, mixed lettuce, romaine lettuce, sweet corn, slicing tomato, beefsteak tomato, hemp, berries, summer squash, edamame, and turf. Locations ranged from across diverse geographical soil and soilless media types, using application methods consistent with standard farmer practices, such as knifing, drip fertigation, soil drench, and combination approaches. All research trials reported no phytotoxicity. One greenhouse trial with tomato and the experimental plan is depicted in FIGS. 12A and 12B. The field trial was conducted in Winter Garden, Florida (Hyperthermic, uncoated Lamellic Quartzipsamments). The treatment plots were 5′×25′ in dimension. Yield (as shown in FIG. 12A) and quality (brix, pH, color, size; plants depicted in FIG. 12B) of tomato fruits were assessed over three harvest periods. Overall, synthetic fertilizer: biofertilizer blends maintained comparable yield as synthetic fertilizer (as demonstrated in FIG. 12A), and no phytotoxicity was observed.

### Example 10. Soil Leaching Trials

Results from soil column leaching trials in collaboration with the International Fertilizer Development Center (IFDC) revealed lower rates of nitrate leaching in field soil treated with biofertilizer. Biofertilizer leached smaller amounts over time (shown as cumulative leached mg Nitrate-N) when applied alone in both low and high rates, and when applied in combination with synthetic conventional and organic fertilizers, such as Calcium Nitrate and Urea.

### Soil Column Analysis

Brownfield sandy loam (loamy, mixed, superactive, thermic Arenic Aridic Paleustalfs), pH 7.7, was used to generate two soil treatments: (1) organically-amended soil and (2) chemically fertilized soil for the leaching study. Organically-amended Brownfield soil consisted of fly larvae manure (2.0% N) at the rate of 4.0 g/kg soil. Fertilized Brownfield (FB) soil consisted of 100 mg P/kg soil, 126 mg K/kg soil applied as monopotassium phosphate (KH₂PO₄) solution, and micronutrient solution as given in Table 6. Organic amendment was added to the topsoil in the column and lightly incorporated into the top 5 cm of the organically amended Brownfield (OAB) soil. Nitrogen sources included (i) the biofertilizer of the present disclosure (BF), (ii) Urea, (iii) BF and urea (1:1), (v) BF and calcium nitrate (1:1), (v) Calcium nitrate, and (vi) no N (Check). Nitrogen was applied at both 92 and 184 mg N/column. Calcium nitrate treatment was applied only at the low rate of 92 mg N/column. 60 columns were analyzed (2 soils×(4 N sources×2 N rates+check+calcium nitrate at 92 mg N)×3 replications (see Table 7)).

Leaching columns were used to quantify N leaching emanating from BF, urea, calcium nitrate, mixture of BF and urea (1:1) and BF+calcium nitrate (1:1) relative to a check. Experimental soils were air-dried and passed through a 2 mm sieve. Each column (5 cm id and 50 cm length) contained 1.5 kg of soil. Field capacity moisture content of Brownfield soil was at 13.22%. Each column was attached to a Buchner funnel (lined with polypropylene mesh) and covered to reduce moisture loss, with the option for maximum vacuum suction of 10 kPa (0.1 bar) that drains leachate into glass jars. Each of the columns was brought to saturated moisture content by adding 50 mL of water daily (equivalent to 25.4 mm/day (1 inch/day) reflecting high rainfall regime) until the columns began to leach. The weight of each column was recorded immediately after leaching stopped; this was considered the saturated moisture content. Additional suction (0.1 bar) was applied for 10 minutes to bring columns below saturation but above the field moisture capacity. The above steps were repeated for 3-4 days to stabilize the columns before N fertilizer application.

The weight of each column (after 10-minute suction) was recorded as the target for the leaching columns. N fertilizers (BF, urea, calcium nitrate, and no N Check) was surface applied at 92 and 184 mg N per column. Immediately after fertilizer application, water was added onto the soil surface equivalent to 20 mL day 1 for three days, followed by 0.1 bar suction after each leaching event. The leachate from each column was quantified and analyzed for urea-N, NH₄—N, and NO₃—N. No additional water was added to these columns for the next 6 days. On day 10, columns were re-watered with a known/measured amount of water that was added until leaching began. The leaching process continued for the next 16 days. The cycle (7-9 days of drying followed by 24 days of leaching) was followed until day 86. Analysis: Measurements for urea-N, NH₄—N, and NO₃—N of the leachates from all the treatments were done at specified intervals.

TABLE 6
Blanket Micronutrient Solution Applied to Fertilized Brownfield
(FB) Soil Columns (Treatments 10-18) at 10 mL

| Amount of Product | | | | |
|---|---|---|---|---|
| (g) into | | Nutrient Added | | |
| 1 L of Water | Chemical | Nutrient | ppm | mg/1.5-kg pot |
| 36.5 | MgSO₄•7H₂O | Mg | 24 | 36 |
| 1.18 | CuSO₄•5H₂O | Cu | 2 | 3 |
| 2.24 | FeSO₄•7H₂O | Fe | 3 | 4.5 |
| 0.92 | MnSO₄•H₂O | Mn | 2 | 3 |
| 0.62 | Na₂B₄O₇•10H₂O | B | 0.5 | 0.75 |
| 0.04 | Na₂MoO₄•2H₂O | Mo | 0.05 | 0.075 |
| 1 mL | H₂SO₄ | | | |

TABLE 7
N and Soil Treatments for Leaching on Organically Amended
Brownfield (OAB) and Brownfield (FB) Sandy Loam.

| | | | Rates | Product | | Product |
|---|---|---|---|---|---|---|
| Trt. | | | (mg | Concentration | | (g/ |
| No. | Soil | N Sources | N/column) | (mg/ml or %) | | column) |
| 1 | OAB | Check | 0 | 0 | 0 | 0.00 |
| 2 | OAB | Urea | 92 | 46% | 0 | 0.20 |
| 3 | OAB | BF | 92 | 0 | 23 | 0.00 |
| 4 | OAB | Ca(NO₃)₂ | 92 | 17% | 0 | 0.53 |
| 5 | OAB | Urea + BF | 92 | 46% | 23 | 0.10 |
| 6 | OAB | Ca(NO₃)₂ + BF | 92 | 17% | 23 | 0.27 |
| 7 | OAB | Urea | 184 | 46% | 0 | 0.40 |
| 8 | OAB | BF | 184 | 0 | 23 | 0.00 |
| 9 | OAB | Urea + BF | 184 | 46% | 23 | 0.20 |
| 10 | OAB | Ca(NO₃)₂ + BF | 184 | 17% | 23 | 0.54 |
| 11 | FB | Check | 0 | 0 | 0 | 0.00 |
| 12 | FB | Urea | 92 | 46% | 0 | 0.20 |
| 13 | FB | BF | 92 | 0 | 23 | 0.00 |
| 14 | FB | Ca(NO₃)₂ | 92 | 17% | 0 | 0.53 |
| 15 | FB | Urea + BF | 92 | 46% | 23 | 0.10 |
| 16 | FB | Ca(NO₃)₂ + BF | 92 | 17% | 23 | 0.27 |
| 17 | FB | Urea | 184 | 46% | 0 | 0.40 |
| 18 | FB | BF | 184 | 0 | 23 | 0.00 |
| 19 | FB | Urea + BF | 184 | 46% | 23 | 0.20 |
| 20 | FB | Ca(NO₃)₂ + BF | 184 | 17% | 23 | 0.54 |

Analysis of variance indicates that product, time of leaching (day), and their interaction, significantly affected daily nitrate-N leaching loss (Table 8). During the pre-fertilizer phase (−10 to 0 day after N application), the variability among columns was attributed to the differences in column packing and settling of soil. After fertilizer application for the first few leachate (days 6-10), there was no significant difference among products as the leachate during this period was not influenced by N fertilization. Much of the differences among products were observed during 13-41 days after N application. From days 13-14 onwards, calcium nitrate (92 mg N) and high rate of urea (184 mg N) application had significantly higher leaching loss than BF. During the early phase, at low rates urea had lower nitrate-N leaching loss than calcium nitrate. This is expected as conversion of urea to nitrates takes several days. However, from day 22 onwards, nitrate losses from urea were significantly greater than from calcium nitrate. Throughout the study period, leaching loss from BF at both rates of application was similar to Check, indicating that BF application did not result in increased nitrate leaching loss. The high rate of BF had higher leaching losses than the low application rate; however, the differences were not significant. Further, when comparing low application rate of urea and calcium nitrate (92 mg N) with high rate of urea+BF (1:1) and calcium nitrate+BF (1:1) at 184 mg N, hence same rate of fertilizer N, leaching losses were not significantly different. This reconfirms that BF did not contribute significantly towards leaching loss. Leaching losses from all products were not significantly different after 42 days and beyond.

TABLE 8
Analysis of Variance for Daily Nitrate-N Leaching
Loss (mg/day) on Organically Amended Brownfield Soil
Type III Tests of Fixed Effects

| Effect | Num DF | Den DF | F Value | Pr > F |
|---|---|---|---|---|
| DAY | 39 | 742 | 87.59 | <.0001 |
| Product | 9 | 150.6 | 26.51 | <.0001 |
| Product*DAY | 351 | 609.6 | 2.71 | <.0001 |

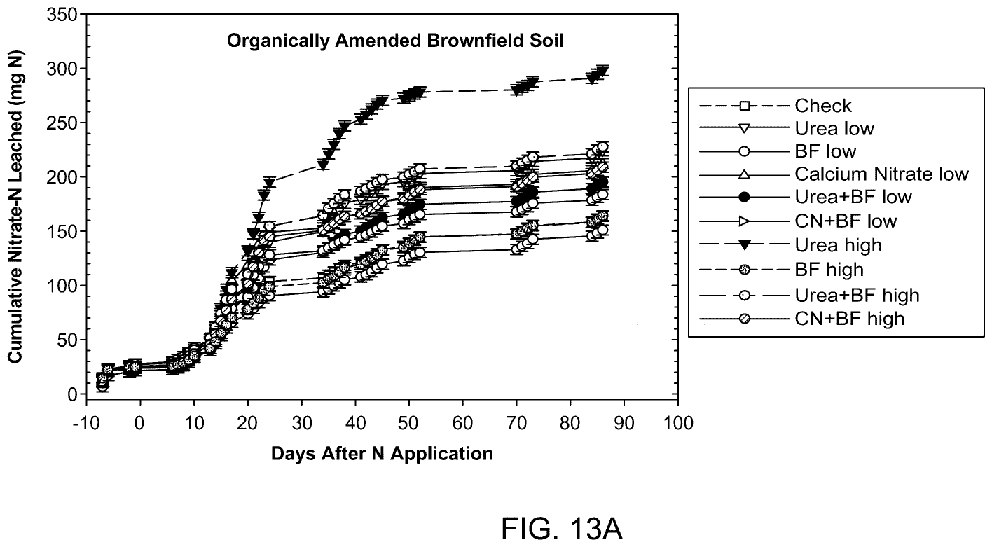

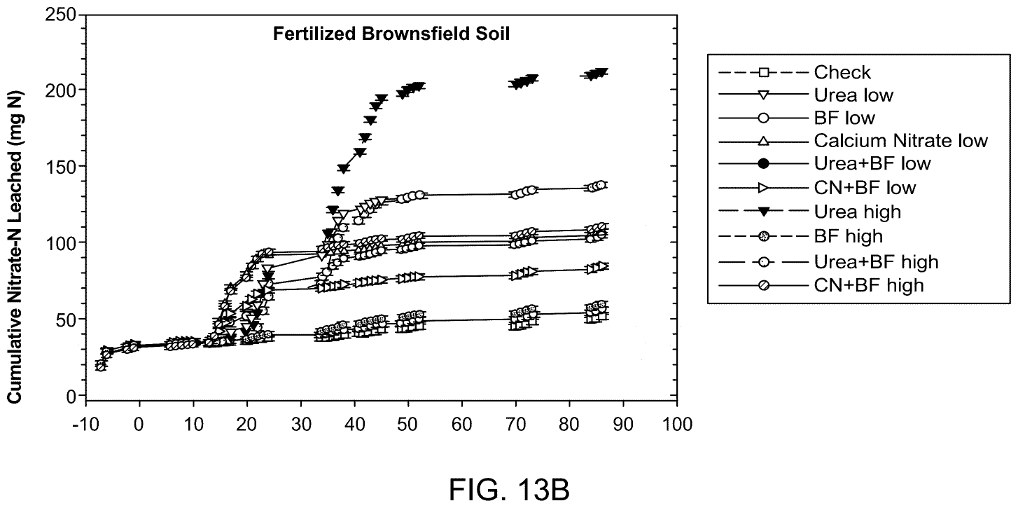

As evident from the analysis of variance (Table 9), cumulative nitrate losses were significantly different among the N products, duration, and duration by product interaction. The leachate collected during the first 25 days (from 10-15 days after N fertilizer application), had no significant difference in nitrate-N content for all products. Cumulative nitrate leaching loss from calcium nitrate and urea fertilizers remained significantly higher than Check and BF treatments from day 18 onwards (FIG. 13A). As shown in Table 10, at 24 days after N application, all urea and calcium nitrate products has significantly higher leaching losses than BF and Check. This trend continued to the end of the study (FIG. 13A, Table 10). High nitrate leaching from Check (and BF only treatments) can be attributed to the application of organic amendments. From 52.5-72.9% of applied N from N fertilizers (urea, calcium nitrate) was lost via leaching and when 50% of N was replaced by BF, total leaching loss was 21.2-34.7% (Table 10). Overall nitrate-N leaching loss from BF application was negligible.

TABLE 9
Analysis of Variance for Cumulative Nitrate-N Leaching
Loss (mg/column) on Organically Amended Brownfield Soil.
Type III Tests of Fixed Effects

| Effect | Num DF | Den DF | F Value | Pr > F |
|---|---|---|---|---|
| DAY | 39 | 777.3 | 543.06 | <.0001 |
| Product | 9 | 26.01 | 38.81 | <.0001 |
| Product*DAY | 351 | 739.6 | 5.97 | <.0001 |

TABLE 10
Comparison of Cumulative Mean Nitrate-N Leaching Loss (mg N and % N
applied) at 24 and 96 Days after N Application. (Differences are not
significant for means followed by the same letter at 5% level).

| | 24 Days | | 86 Days | |
|---|---|---|---|---|
| | Mean | Mean (% N | Mean | Mean (% N |
| | (mg N) | Applied) | (mg N) | Applied) |
| Urea (184 mg N) | 195.3 A | 49.8 | 298.1 A | 72.9 |
| Urea (92 mg N) + BF (92 | 154.6 B | 27.7 | 227.7 B | 34.7 |
| mg N) | | | | |
| Calcium Nitrate (92 mg N) | 149.1 BC | 49.5 | 212.2 CD | 52.5 |
| Calcium Nitrate (92 mg N) + | 145.0 BC | 22.5 | 209.0 D | 24.5 |
| BF (92 mg N) | | | | |
| Urea (92 mg N) | 139.9 CD | 39.5 | 224.1 BC | 65.4 |
| Calcium Nitrate (46 mg N) + | 128.1 DE | 26.6 | 183.4 E | 21.2 |
| BF (46 mg N) | | | | |
| Urea (46 mg N) + BF (46 | 123.5 E | 21.6 | 195.3 E | 34.1 |
| mg N) | | | | |
| Check (0 N) | 103.6 F | 0.0 | 163.9 FG | 0.0 |
| BF (184 mg N) | 98.9 FG | −2.6 | 164.2 F | 0.2 |
| BF (92 mg N) | 90.5 G | −14.2 | 151.1 G | −13.9 |

Analysis of variance for fertilized Brownfield soil indicated that product, time of leaching (day), and their interaction, significantly affected daily nitrate-N leaching loss (Table 11). Differences due to N products appeared from 13 days after N application. At this stage, calcium nitrate products had significantly higher nitrate leaching loss than urea. Addition of BF (92 mg N) to calcium nitrate (92 mg N) had no significant difference in leaching losses compared to calcium nitrate low treatment alone at 92 mg N during 13-20 days after N application. Similar conclusion can be drawn from comparing calcium nitrate low (92 mg N) with calcium nitrate (46 mg N)+BF (46 mg N) low treatment where losses were significantly higher when all N was supplied by calcium nitrate. Nitrate leaching loss from urea-based fertilizer was delayed (from day 20 onwards) as expected due to transformation of urea first into ammonium-N and then nitrate-N. Again, BF did not contribute towards nitrate leaching loss. In this unamended Brownfield soil, nitrate leaching loss from Check (no N fertilization) was negligible with major losses occurring on treatments with N fertilizer application. The intensity of leaching was intense and by day 60 most of the applied N fertilizer was leached as evident from low leachate N content during the last 20 days of the leaching study.

TABLE 11
Analysis of Variance for Daily Nitrate-N Leaching
Loss (mg/day) on Fertilized Soil.
Type III Tests of Fixed Effects

| Effect | Num DF | Den DF | F Value | Pr > F |
|---|---|---|---|---|
| DAY | 39 | 740.9 | 236.86 | <.0001 |
| Product | 9 | 194.2 | 111.98 | <.0001 |
| Product*DAY | 351 | 608.6 | 11.47 | <.0001 |

Cumulative nitrate losses on fertilized Brown field soil were also significantly different among the N products, duration, and duration by product interaction (Table 12). There were no significant differences in cumulative nitrate leaching loss during the first 23 days (−10 to 13 days after N application) of the study (FIG. 13B). Thereafter significant differences were observed among many of the treatments. As shown in Table 13, nitrate leaching loss from calcium nitrate at 24 days after N application was significantly higher than with the equivalent rates of urea application. When 50% of N supply was replaced by BF for urea or for calcium nitrate, leaching losses were significantly lower as shown by cumulative nitrate leaching loss at 24 and 86 days after N application (FIG. 13B, Table 13). Losses from BF and Check (without any N application) were negligible with no significant differences among each other. At 24 days after N application, 58.8% of applied N from calcium nitrate was lost due to leaching compared to 1.4% with BF. Cumulative leaching loss at the end of the study was highest for urea at 87.6-94.2% compared with 4-4.8% with BF.

TABLE 12
Analysis of Variance for Cumulative Nitrate-N Leaching
Loss (mg/day) on Fertilized Soil.
Type III Tests of Fixed Effects

| Effect | Num DF | Den DF | F Value | Pr > F |
|---|---|---|---|---|
| DAY | 39 | 740.9 | 236.86 | <.0001 |
| Product | 9 | 194.2 | 111.98 | <.0001 |
| Product*DAY | 351 | 608.6 | 11.47 | <.0001 |

TABLE 13
Comparison of Cumulative Mean Nitrate-N Leaching Loss (mg N and % N applied)
at 24 and 96 Days after N Application on Fertilized Brownfield Soil. (Differences
are not significant for means followed by the same letter at 5% level)

| | 24 Days | | | 86 Days | | |
|---|---|---|---|---|---|---|
| | Mean | | Mean (% N | Mean | | Mean (% N |
| | (mg N) | | Applied) | (mg N) | | Applied) |
| Urea (184 mg N) | 77.4 | C | 22.1 | 212.0 | A | 87.6 |
| Urea (92 mg N) | 82.3 | B | 49.6 | 137.5 | B | 94.2 |
| Urea (92 mg N) + BF (92 mg | 63.1 | DE | 14.3 | 137.3 | B | 47.0 |
| N) | | | | | | |
| Calcium Nitrate (92 mg N) + | 92.5 | A | 30.3 | 109.6 | C | 32.0 |
| BF (92 mg N) | | | | | | |
| Calcium Nitrate (92 mg N) | 90.9 | A | 58.9 | 106.0 | CD | 60.0 |
| Urea (46 mg N) + BF (46 mg | 62.1 | E | 27.6 | 101.6 | D | 55.2 |
| N) | | | | | | |
| Calcium Nitrate (46 mg N) + | 66.8 | D | 32.7 | 83.5 | E | 35.5 |
| BF (46 mg N) | | | | | | |
| BF (184 mg N) | 39.0 | F | 1.3 | 58.2 | F | 4.0 |
| BF (92 mg N) | 38.0 | F | 1.4 | 55.2 | FG | 4.8 |
| Check (0 N) | 36.7 | F | 0.0 | 50.8 | F | 0.0 |

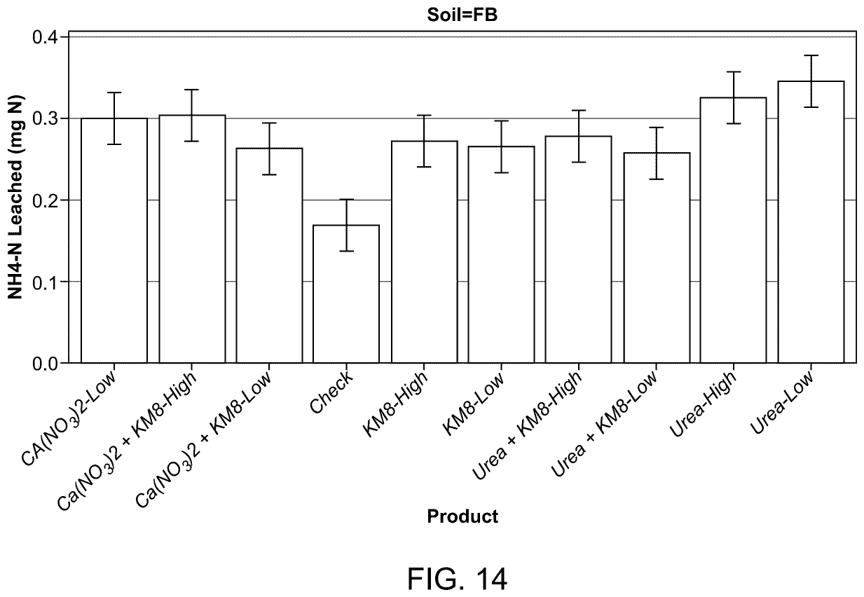

As shown in the analysis of variance table, ammonium-N leaching losses were not significantly different among N products when applied to the organically amended Brownfield soil (Table 14). However, significant differences were observed in ammonium-N leaching losses among some products on fertilized Brownfield soil (Table 15, FIG. 14). As expected, the magnitude of ammonium-N leaching loss was negligible compared to nitrate leaching loss. All products, including BF had significantly higher ammonium-N leaching loss than Check (FIG. 14).

TABLE 14
Analysis of Variance for Cumulative Ammonium-N Leaching Loss
(mg/day) on Organically Amended Brownfield Soil.
Type III Tests of Fixed Effects

| Effect | Num DF | Den DF | F Value | Pr > F |
|---|---|---|---|---|
| DAY | 39 | 740.9 | 236.86 | <.0001 |
| Product | 9 | 194.2 | 111.98 | <.0001 |
| Product*DAY | 351 | 608.6 | 11.47 | <.0001 |

TABLE 15
Analysis of Variance for Cumulative Ammonium-N Leaching
Loss (mg/day) on Fertilized Brownfield Soil
Type III Tests of Fixed Effects

| Effect | Num DF | Den DF | F Value | Pr > F |
|---|---|---|---|---|
| DAY | 39 | 740.9 | 236.86 | <.0001 |
| Product | 9 | 194.2 | 111.98 | <.0001 |
| Product*DAY | 351 | 608.6 | 11.47 | <.0001 |

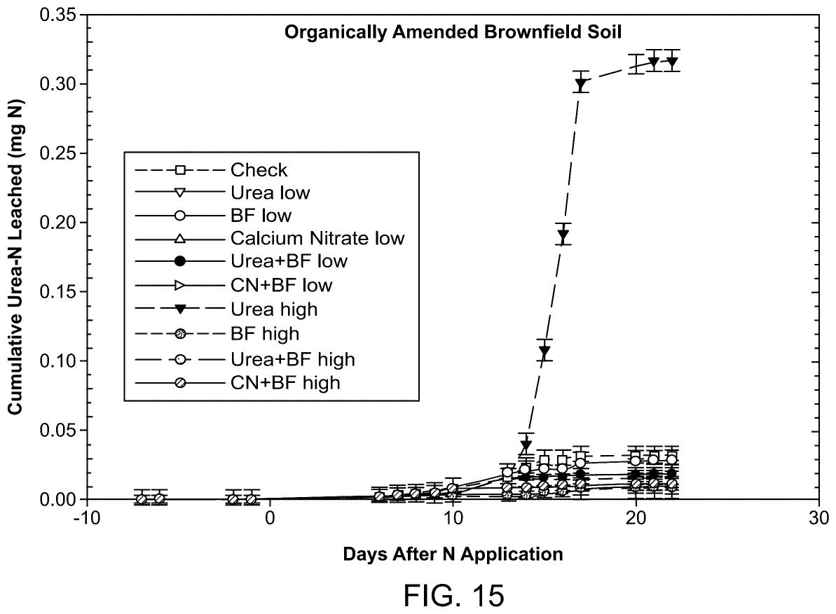

Only at the highest urea application rate on organically amended soil, urea-N leaching losses were observed (FIG. 15). On the fertilized (non-organic amended soil), urea-N leaching loss was not significantly different from untreated (Check) soil.

### Soil Incubation

Laboratory incubation experiments were carried out to measure and compare rates of N transformation and the changes in soil acidity levels of selected soils when applied with BF compared with urea and calcium nitrate. Each soil was pre-incubated for 12 days at 75% field moisture content before nitrogen treatment application. All pre-incubation soil had other fertilizers (except N) or organic amendment already applied.

Three soils—Lakeland sand (pH 4.5), Greenville loamy soil (pH 6.2) and Brownfield loamy sand (pH 7.7) were used for the incubation study. N Sources and amendments (12): (i) BF (organically amended), (ii) BF (fertilized soil), (iii) BF+Urea (organic amendment), (iv) BF+urea (fertilized soil), (v) Urea (organic amendment), (vi) Urea (fertilized soil), (vii) No N (organic amendment), (viii) No N (fertilized soil), (ix) BR+Calcium nitrate (organic amendment) (x) BR+Calcium nitrate (fertilized soil), (xi) Urea+Calcium nitrate (organic amendment), and (xii) Urea+Calcium nitrate (fertilized soil).

The 12 treatments in Table 16 were repeated for each of the three soils (Lakeland, Greenville, and Brownfield). Organic amendment consisted of fly larvae manure (2.0% N) applied at 10.0 g/kg soil or 0.5 g manure per 50 g air-dry soil. Fertilized Brownfield soil consisted of 100 mg P/kg soil, 126 mg K/kg soil applied as monopotassium phosphate (KH₂PO₄) solution (1 mL per cup), and micronutrient solution (1 mL per cup) as given in Table 17. The fertilized soil treatment received 2 mL fertilizer solution per 50 g soil (incubation cup). Additional water was added to both organically amended and fertilized soil cups so that the final moisture content of all the pre-incubation cups for each of the three soils (Lakeland, Greenville, and Brownfield) was at 75% field moisture capacity. After 12 days of pre-incubation, N products (urea, calcium nitrate, BF) were applied as per treatment (Table 17). To the pre-incubated soil sample cups containing fifty grams (50 g; air-dry equivalent) of soil (organically amended, fertilized), 10 mg N were added and incubated for a period of 17 weeks. Equivalent amounts of urea, calcium nitrate, and BF were applied as solution to reach the target N rate of 10 mg N per cup (Table 18). Note: the amount of calcium nitrate applied was 3.47 mg N per cup instead of 5 mg N, hence total N application for treatment cups with calcium nitrate was only 8.47 mg N (Table 16). During the entire incubation period, moisture content for each cup was maintained at 75% field moisture capacity and kept at constant temperature of 25° C. The total number of incubation cups was 1080 with 12 treatments (Table 16), three soils, three replication and 10 incubation sampling period. To avoid error associated with subsampling for a given incubation time, separate incubation cups were setup for the 10 different incubation times at 0, 1, 2, 3, 4, 6, 8, 10, 12, and 17 weeks after N application. Total number of cups=12 treatments×3 soils×3 replication×10 incubation period=1080. After each incubation period, measurements were made for NH₄⁺-N, and NO₃⁻-N by 2 M KC extraction to quantify the rates of N transformation of the fertilizer N products. During the 0, and 1-week incubation period urea-N will also be analyzed, if urea-N is present beyond 7 days, then 14-day incubation sample was also analyzed. For the 0-, 6- and 17-week incubation samples, soil pH (1:1 soil:water) was determined first.

TABLE 15
Treatment Detail for N Products Applied to Organically
Amended (O) and Fertilized (F) Soil

| | | | N Rates | Weight of N | Volume |
|---|---|---|---|---|---|
| Trt. | | | (mg | Fertilizer Product | of BF |
| No. | Soil | N Sources | N/cup) | (mg) | (mL) |
| 1 | O | Check | 0 | 0 | 0 |
| 2 | O | Urea | 10 | 21.7 | 0 |
| 3 | O | BF | 10 | 0 | 0.44 |
| 4 | O | BF + Urea (1:1) | 10 | 10.85 | 0.22 |
| 5 | O | BF + Calcium Nitrate (1:1) | 8.47 | 29.29 | 0.22 |
| 6 | O | Urea + Calcium Nitrate | 8.47 | 10.85/29.29 | 0 |
| 7 | F | Check | 10 | 0 | 0 |
| 8 | F | Urea | 10 | 21.7 | 0 |
| 9 | F | BF | 10 | 0 | 0.44 |
| 10 | F | BF + Urea (1:1) | 10 | 10.85 | 0.22 |
| 11 | F | BF + Calcium Nitrate (1:1) | 8.47 | 29.29 | 0.22 |
| 12 | F | Urea + Calcium Nitrate | 8.47 | 10.85/29.29 | 0 |

Notes:
1. BF + urea (Treatments 4 and 10) have 50% of N requirement (5 mg N) met by BF.
2. BF + Ca(NO₃)₂ (Treatments 5 and 11) have 50% of N requirement (5 mg N) met by BF.
3. Calcium nitrate in Treatments 5, 6, 11, and 11 provided only 3.47 mg N - hence total N applied was only 8.47 mg N instead of 10 mg N as with other treatments.

TABLE 16
Blanket Micronutrient Solution Applied to “Fertilized
Soil” Cups (Treatments 7-12) at 1 mL.

| Amount of | | | | |
|---|---|---|---|---|
| Product (g) into | | Nutrient Added | | |
| 1 L of Water | Chemical | Nutrient | ppm | mg/50 g cup |
| 12.17 | MgSO₄•7H₂O | Mg | 24 | 1.2 |
| 0.394 | CuSO₄•5H₂O | Cu | 2 | 0.1 |
| 0.746 | FeSO₄•7H₂O | Fe | 3 | 0.15 |
| 0.308 | MnSO₄•H₂O | Mn | 2 | 0.1 |
| 0.220 | Na₂B₄O₇•10H₂O | B | 0.5 | 0.025 |
| 0.0063 | Na₂MoO₄•2H₂O | Mo | 0.05 | 0.0025 |
| 1 mL | H₂SO₄ | | | |

TABLE 17
N Product Solution Preparation.
Kula Bio DILUTED Solution Preparation - Incubation

| Volume/Cup | Amount of DILUTED | Amt of STOCK (mL) to | |
|---|---|---|---|
| of Stock | Solution to be Applied/ | Make 500 mL DILUTED | Treatments to |
| Solution (mL) | Cup (mL) | Solution | be Applied |
| 0.22 | 1 | 110 | 4, 5, 10 and 11 |
| 0.44 | 1 | 220 | 3 and 9 |

UREA Solution Preparation - Incubation

| Amount/Cup | Amount of DILUTED | Amt of Urea (g) to Make | |
|---|---|---|---|
| of Stock | Solution to be Applied/ | 500 mL DILUTED | Treatments to |
| Solution (mg) | Cup (mL) | Solution | be Applied |
| 10.85 | 1 | 5.425 | 4, 6, 10 and 12 |
| 21.7 | 1 | 10.85 | 2 and 8 |

Calcium Nitrate Solution Preparation - Incubation

| Amount per | Amount of DILUTED | Amt of Calcium Nitrate | |
|---|---|---|---|
| cup of stock | solution to be applied | (g) to make 500 mL | Treatments to |
| Solution (mg) | per cup (mL) | DILUTED solution | be applied |
| 29.29 | 1 | 14.65 | 5, 6, 11 and 12 |

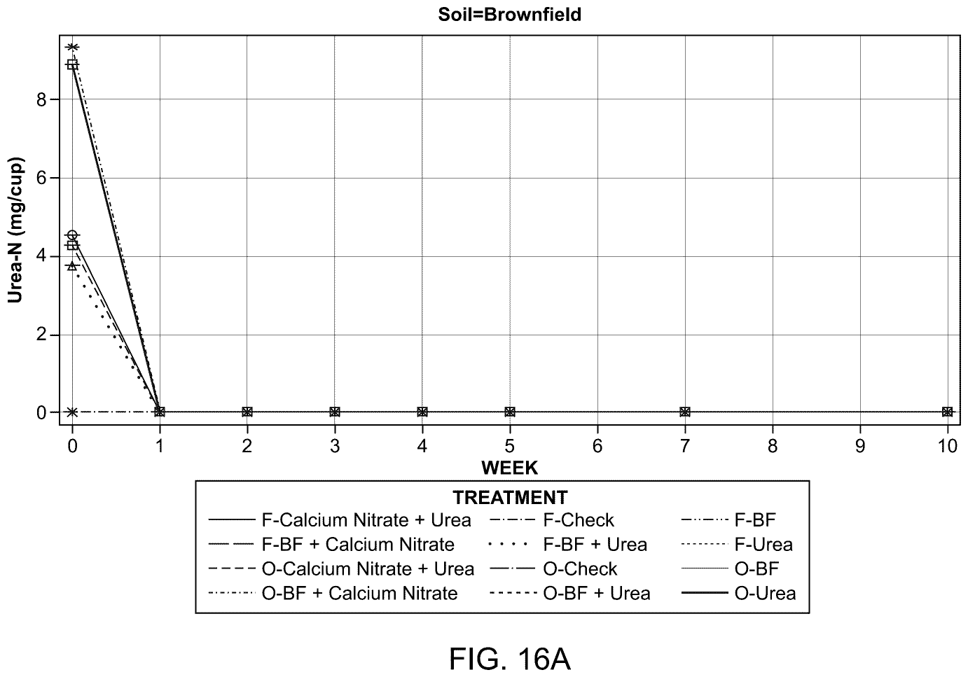

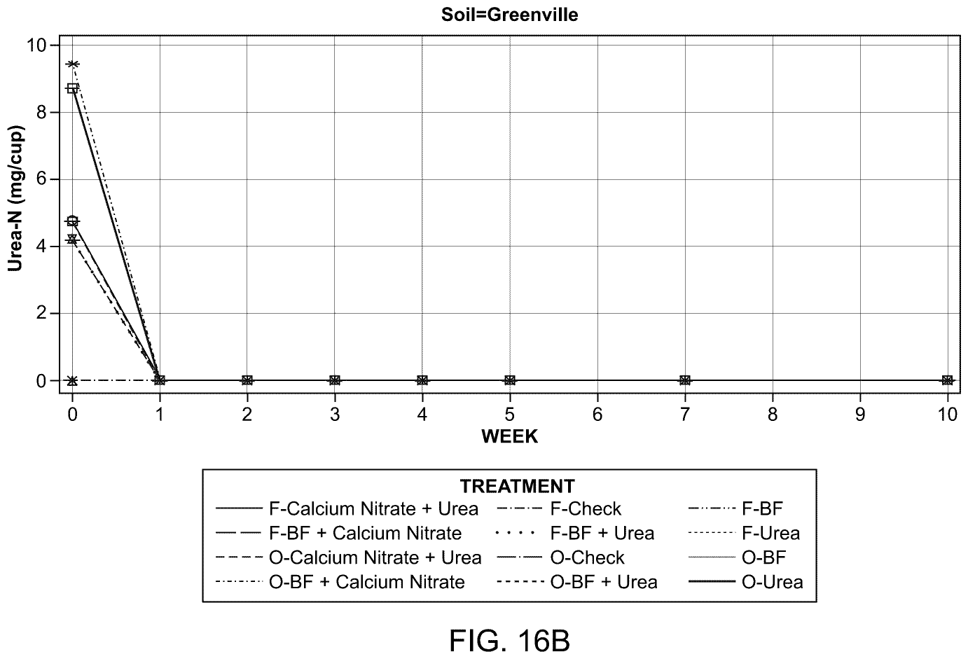

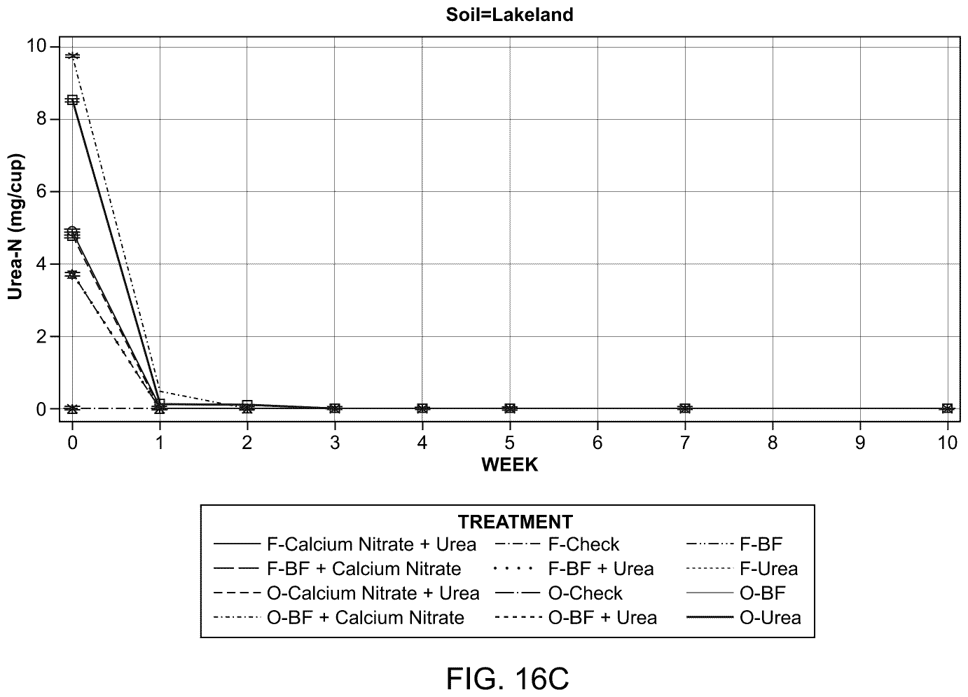

Analysis of variance showed significant differences among treatments (N sources and organic amendment vs fertilized soil), time of sampling and treatment by time interaction for urea-N content on all three soils—Brownfield, Greenville, and Lakeland (Table 18). Urea-N content was detected only on soils applied with urea (FIGS. 16A-16C). However, within a week after application, all urea had hydrolyzed to ammonium-N except in the Lakeland sandy soil. The Lakeland soil with lower pH and low soil organic matter content had lower urea hydrolysis rate. As expected, at zero-time soil urea-N content was the highest for the treatments where all 10 mg N was applied as urea.

TABLE 18
Analysis of Variance for Urea-N Dynamics

| | | | Brownfield Soil | | Greenville Soil | | Lakeland | |
|---|---|---|---|---|---|---|---|---|
| | Num | Den | F | Pr > | F | Pr > | F | Pr > |
| Effect | DF | DF | Value | F | Value | F | Value | F |
| WEEK | 7 | 164 | 91765.5 | <.0001 | 69093.8 | <.0001 | 11675.6 | <.0001 |
| Treatment | 11 | 164 | 19007.8 | <.0001 | 14010.4 | <.0001 | 2490.70 | <.0001 |
| WEEK* | 63 | 164 | 9476.98 | <.0001 | 6766.64 | <.0001 | 1200.96 | <.0001 |
| Treatment | | | | | | | | |

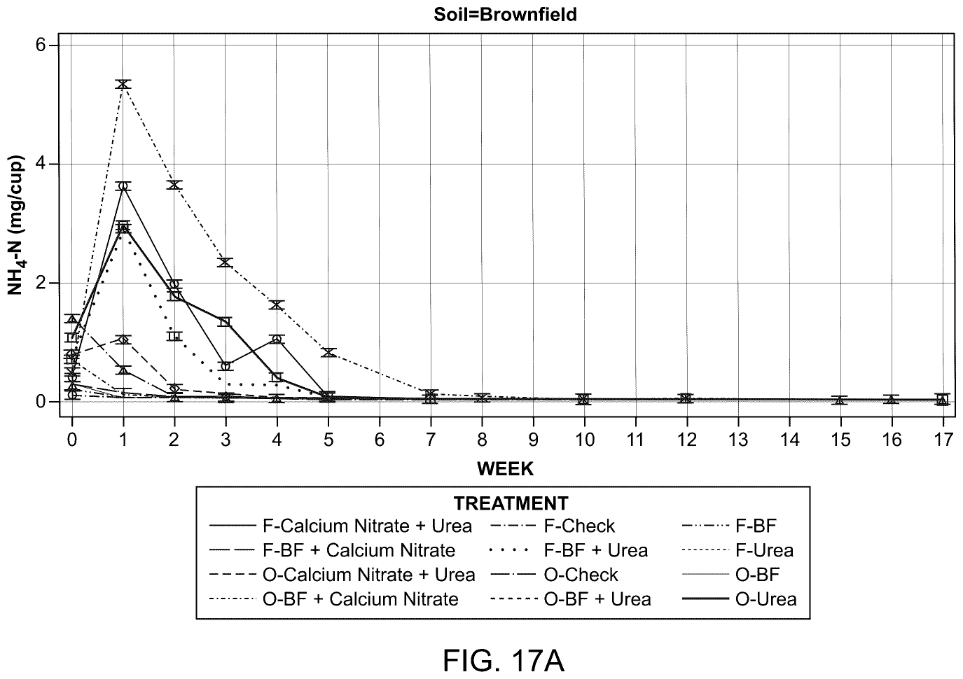

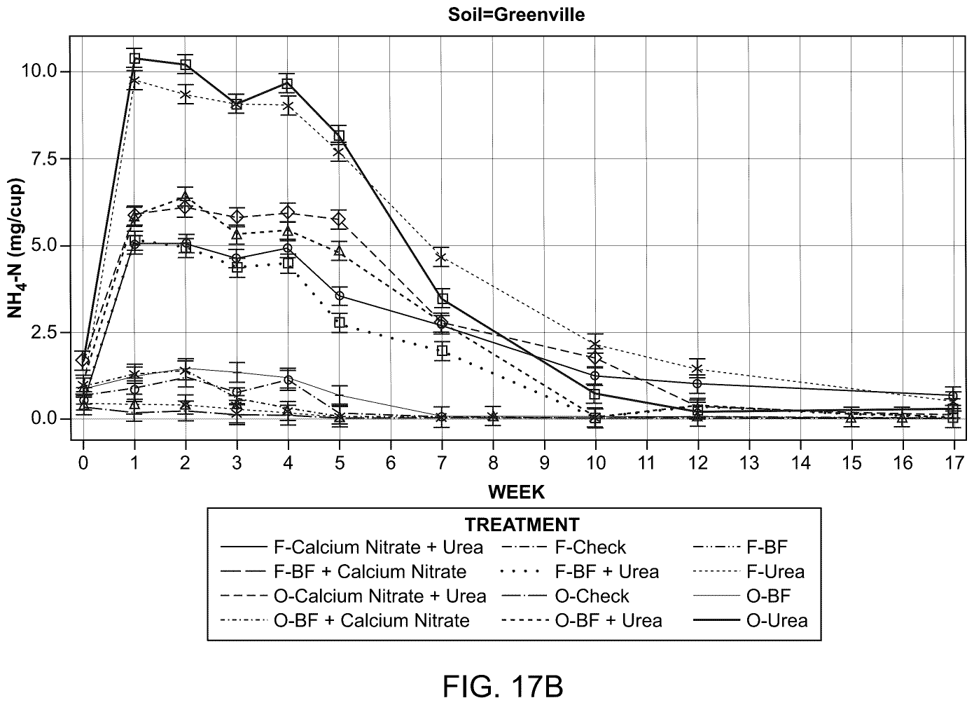

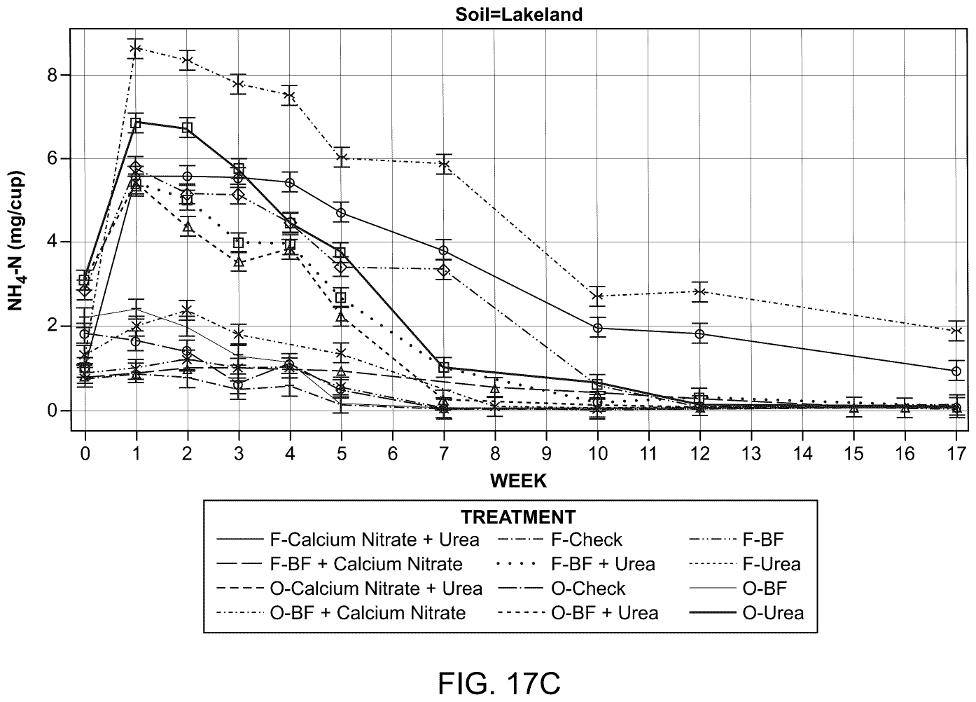

Analysis of variance showed that treatments (N sources and organic amendment versus fertilizer), time (day) and their interaction were significantly different on all soils (Table 19). On Brownfield soil, at time-zero, the organically amended treatments had higher ammonium-N content than the corresponding treatments on fertilized soil (FIG. 17A). These differences were highly significant except for Check (no N) and BF application. During the entire incubation period, there was no significant difference between soil NH₄—N content of Check and BF on organically amended and fertilized Brownfield soil. During weeks 1 through 5, fertilized soil with any urea application had significantly higher NH₄—N content than the organically amended Brownfield soil. All the differences among treatments can be attributed to urea application. As expected, calcium nitrate did not supply any NH₄—N and throughout the incubation period there was no significant contribution from BF. Overall soil NH₄—N content was the highest for fertilized urea>fertilized urea+calcium nitrate>organically amended urea>fertilized urea+BF. From week 7 onwards there was no significant difference among all N products independent of organic manure or fertilizer application (FIG. 17A). However, this was not the case on Greenville or Lakeland soils where differences in NH₄—N content was observed up to 12-17 weeks after N application (FIGS. 17B and 17C).

At zero-time, urea products had significantly higher NH₄—N content on Greenville soil amended with organic manure than on fertilized Greenville soil (FIG. 17A). Urea-alone application on fertilized soil gave significantly higher NH₄—N content from week 1 onwards. Similar trend was also observed with urea on organically amended Greenville soil (FIG. 17A). Both BF+urea and calcium nitrate+urea on organically amended Greenville soil had higher soil NH₄—N content than on fertilized soil. Since the nitrification rate was higher on organically amended soil, NH₄—N content declined rapidly from week 7 onwards. As with Brownfield soil, BF application on Greenville soil did not lead to any significant increase in soil NH₄—N content. There was no significant difference in soil NH₄—N content between Check (no N application) and BF or BF+calcium nitrate application (FIG. 17A).

On the acidic (pH 4.5) Lakeland soil, NH₄—N content was significantly higher for fertilized soil than the organically amended soil when comparing the same N sources (FIG. 17B). Significant amount of applied N from urea (up to 20%) remained in NH₄—N form after 17 weeks in the fertilized Lakeland soil compared to organically amended Lakeland soil. Organic amendment promoted higher rate of nitrification by buffering the soil pH. Overall, on fertilized Lakeland soil, BF application did not result in significant increase soil NH₄—N content compared to Check (FIG. 17B, Table 20). However, on Lakeland soil amended with organic manure, BF and/or BF+calcium nitrate gave significantly higher soil NH₄—N content than the Check during weeks 1, 2, 3 and 5 (Table 20).

Based on the combination of three soils and organic amendments versus fertilizers, application of BF did not lead to significant increase in soil ammonium-N content. Some evidence for increase in soil ammonium-N content with BF application was observed on organically amended Lakeland soil.

TABLE 19
Analysis of Variance for NH₄-N Dynamics

| | | | Brownfield Soil | | Greenville Soil | | Lakeland Soil | |
|---|---|---|---|---|---|---|---|---|
| | Num | Den | F | Pr > | F | Pr > | F | Pr > |
| Effect | DF | DF | Value | F | Value | F | Value | F |
| WEEK | 12 | 240 | 412.30 | <.0001 | 305.54 | <.0001 | 318.53 | <.0001 |
| Treatment | 11 | 240 | 438.64 | <.0001 | 512.36 | <.0001 | 435.38 | <.0001 |
| WEEK* | 96 | 240 | 94.29 | <.0001 | 33.70 | <.0001 | 23.80 | <.0001 |
| Treatment | | | | | | | | |

TABLE 20
Pairwise Comparison of NH4-N for Selected Treatments on Organically
Amended (O) and Fertilized (F) Lakeland Soil
Simple Effect Comparisons of DAY*N SOURCE Least Squares Means By DAY

| Simple | | | | | | | |
|---|---|---|---|---|---|---|---|
| Effect | N- | N- | | Standard | | t | Pr > |
| Level | SOURCE | SOURCE | Estimate | Error | DF | Value | \|t\| |
| WEEK 1 | O-Check | O-BF | −0.7441 | 0.3255 | 240 | −2.29 | 0.0231 |
| WEEK 1 | O-Check | O-BF + | −0.3398 | 0.3255 | 240 | −1.04 | 0.2976 |
| | | Calcium | | | | | |
| | | Nitrate | | | | | |
| WEEK 1 | O-Check | O-BF + | −3.7383 | 0.3255 | 240 | −11.48 | <.0001 |
| | | Urea | | | | | |
| WEEK 1 | O-Check | O-Urea | −5.2098 | 0.3255 | 240 | −16.00 | <.0001 |
| WEEK 1 | O-BF | O-BF + | 0.4043 | 0.3255 | 240 | 1.24 | 0.2154 |
| | | Calcium | | | | | |
| | | Nitrate | | | | | |
| WEEK 1 | O-BF | O-BF + | −2.9941 | 0.3255 | 240 | −9.20 | <.0001 |
| | | Urea | | | | | |
| WEEK 2 | O-Check | O-BF | −0.5848 | 0.3255 | 240 | −1.80 | 0.0737 |
| WEEK 2 | O-Check | O-BF + | −0.9739 | 0.3255 | 240 | −2.99 | 0.0031 |
| | | Calcium | | | | | |
| | | Nitrate | | | | | |
| WEEK 2 | O-Check | O-BF + | −2.9841 | 0.3255 | 240 | −9.17 | <.0001 |
| | | Urea | | | | | |
| WEEK 2 | O-Check | O-Urea | −5.3143 | 0.3255 | 240 | −16.33 | <.0001 |
| WEEK 2 | O-BF | O-BF + | −0.3891 | 0.3255 | 240 | −1.20 | 0.2332 |
| | | Calcium | | | | | |
| | | Nitrate | | | | | |
| WEEK 2 | O-BF | O-BF + | −2.3993 | 0.3255 | 240 | −7.37 | <.0001 |
| | | Urea | | | | | |
| WEEK 3 | O-Check | O-BF | −0.6379 | 0.3255 | 240 | −1.96 | 0.0512 |
| WEEK 3 | O-Check | O-BF + | −1.1703 | 0.3255 | 240 | −3.60 | 0.0004 |
| | | Calcium | | | | | |
| | | Nitrate | | | | | |
| WEEK 3 | O-Check | O-BF + | −2.8869 | 0.3255 | 240 | −8.87 | <.0001 |
| | | Urea | | | | | |
| WEEK 3 | O-Check | O-Urea | −5.0960 | 0.3255 | 240 | −15.65 | <.0001 |
| WEEK 3 | O-BF | O-BF + | −0.5324 | 0.3255 | 240 | −1.64 | 0.1033 |
| | | Calcium | | | | | |
| | | Nitrate | | | | | |
| WEEK 3 | O-BF | O-BF + | −2.2490 | 0.3255 | 240 | −6.91 | <.0001 |
| | | Urea | | | | | |
| WEEK 3 | O-BF | O-Urea | −4.4581 | 0.3255 | 240 | −13.70 | <.0001 |
| WEEK 5 | F-Check | F-BF | −0.4120 | 0.3255 | 240 | −1.27 | 0.2069 |
| WEEK 5 | F-Check | F-BF + | −0.7972 | 0.3255 | 240 | −2.45 | 0.0150 |
| | | Calcium | | | | | |
| | | Nitrate | | | | | |
| WEEK 5 | O-Check | O-BF | 0.2957 | 0.3255 | 240 | 0.91 | 0.3646 |
| WEEK 5 | O-Check | O-BF + | −0.8902 | 0.3255 | 240 | −2.73 | 0.0067 |
| | | Calcium | | | | | |
| | | Nitrate | | | | | |
| WEEK 5 | O-Check | O-BF + | −1.7735 | 0.3255 | 240 | −5.45 | <.0001 |
| | | Urea | | | | | |
| WEEK 5 | O-Check | O-Urea | −3.3152 | 0.3255 | 240 | −10.18 | <.0001 |
| WEEK 5 | O-BF | O-BF + | −1.1859 | 0.3255 | 240 | −3.64 | 0.0003 |
| | | Calcium | | | | | |
| | | Nitrate | | | | | |
| WEEK 5 | O-BF | O-BF + | −2.0692 | 0.3255 | 240 | −6.36 | <.0001 |
| | | Urea | | | | | |
| WEEK 7 | O-Check | O-BF | 0.007983 | 0.3255 | 240 | 0.02 | 0.9805 |
| WEEK 7 | O-Check | O-BF + | −0.1968 | 0.3255 | 240 | −0.60 | 0.5461 |
| | | Urea | | | | | |
| WEEK 7 | O-Check | O-Urea | −0.9798 | 0.3255 | 240 | −3.01 | 0.0029 |
| WEEK 7 | O-BF | O-BF + | −0.2048 | 0.3255 | 240 | −0.63 | 0.5299 |
| | | Urea | | | | | |

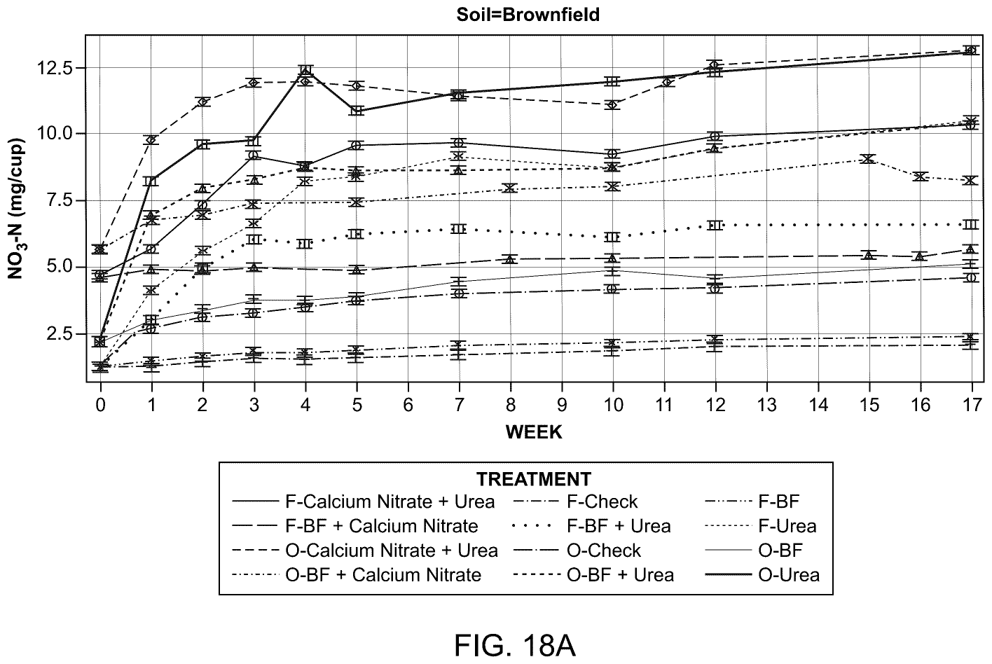

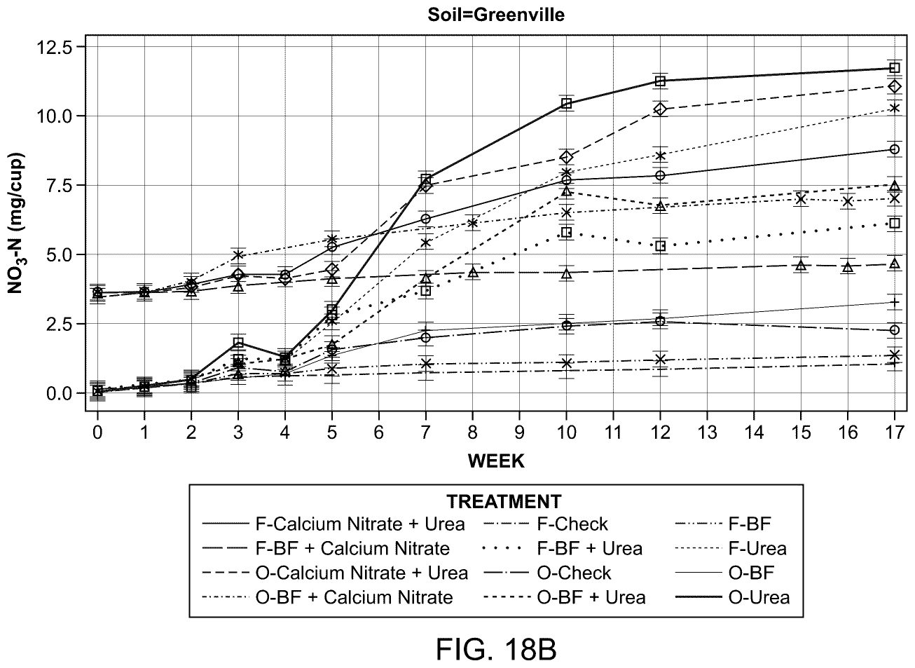

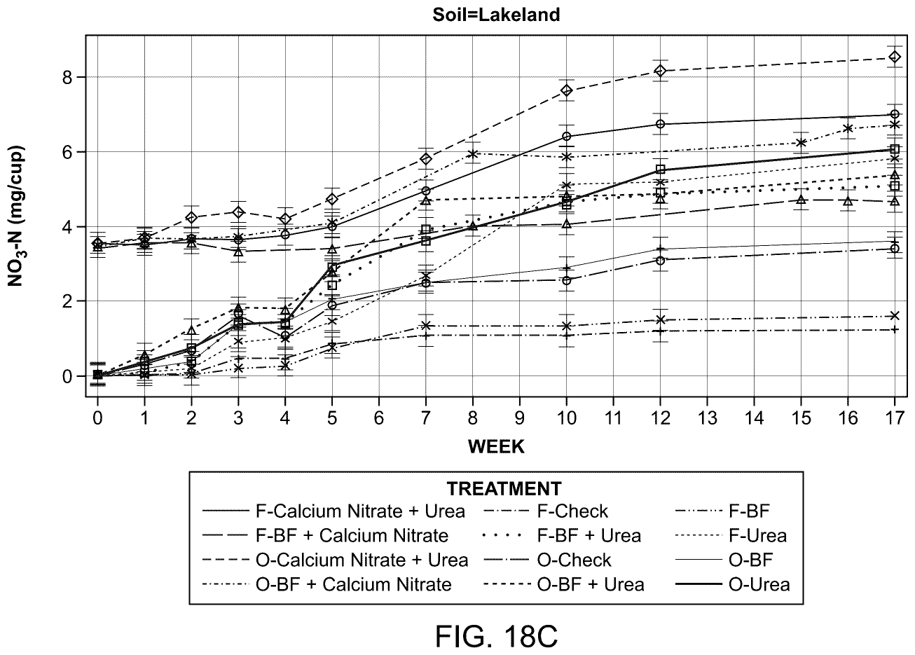

Analysis of variance showed that N products and organic versus chemical fertilizers (Treatments), time (Week) and their interaction had significant effect on soil nitrate-N content of all three soils (Table 21). On all three soils, calcium nitrate application resulted in significantly higher soil nitrate-N during the first three weeks after N application (FIGS. 18A-18C). On organically amended Brownfield soil throughout the 17-week incubation period, soil NO₃—N content was significantly higher than on fertilized soil for all six N treatments including the Check (zero-N). Soil NO₃—N was significantly higher for all treatments on the organically amended Greenville and Lakeland soils compared to fertilizer amended soil from seven weeks after N application (FIGS. 18B and 18C). Overall, nitrification of organic amendment was quicker on the Brownfield soil.

As evident from Table 22, only on organically amended Brownfield and Greenville soils, BF application led to significantly higher soil NO₃—N content than Check. On Lakeland soil, nitrate recovery for all treatments remained below 68%. Interestingly on organically amended Lakeland soil, when 50% of urea was substituted by BF, soil NO₃—N content was not significantly different. This indicates, nitrate-N contribution form BF. On all soils with organic manure or fertilizer amendments, 50% substitution of urea did not result in 50% decline in NO₃—N content (Table 22), this indicates that lower urea-N application was more efficient and/or application of BF contributed towards soil NO₃—N.

TABLE 21
Analysis of Variance for NO3-N Dynamics

| | | | Brownfield Soil | | Greenville Soil | | Lakeland Soil | |
|---|---|---|---|---|---|---|---|---|
| | Num | Den | F | Pr > | F | Pr > | F | Pr > |
| Effect | DF | DF | Value | F | Value | F | Value | F |
| WEEK | 12 | 239 | 669.50 | <.0001 | 432.89 | <.0001 | 224.86 | <.0001 |
| Treatment | 11 | 239 | 3522.27 | <.0001 | 460.37 | <.0001 | 293.79 | <.0001 |
| WEEK* | 96 | 239 | 44.86 | <.0001 | 28.60 | <.0001 | 6.48 | <.0001 |
| Treatment | | | | | | | | |

TABLE 22
Comparison of Soil NO₃—N After 17 Weeks of Incubation for Treatments
(N Products and Amendments - Organic [O] and Fertilized [F])
on Brownfield, Greenville, and Lakeland Soils. (Differences are not
significant for means followed by the same letter at 5% level)).

| SOIL | | Mean (mg N) | | Nitrate Recovery (%) |
|---|---|---|---|---|
| Brownfield | O-Calcium Nitrate + Urea | 13.15 | A | 100.0 |
| | O-Urea | 13.07 | A | 84.7 |
| | F-Urea | 10.52 | B | 84.4 |
| | F-Calcium Nitrate + Urea | 10.32 | B | 97.3 |
| | O-BF + Urea | 10.31 | B | 57.1 |
| | O-BF + Calcium Nitrate | 8.26 | C | 43.2 |
| | F-BF + Urea | 6.61 | D | 45.3 |
| | F-BF + Calcium Nitrate | 5.68 | E | 42.5 |
| | O-BF | 5.12 | F | 5.2 |
| | O-Check (No N) | 4.60 | G | 0.0 |
| | F-BF | 2.37 | H | 2.9 |
| | F-Check (No N) | 2.08 | H | 0.0 |
| Greenville | O-Urea | 11.72 | A | 94.7 |
| | O-Calcium Nitrate + Urea | 11.06 | A | 103.6 |
| | F-Urea | 10.28 | B | 92.2 |
| | F-Calcium Nitrate + Urea | 8.79 | C | 91.3 |
| | O-BF + Urea | 7.51 | D | 52.6 |
| | O-BF + Calcium Nitrate | 7.02 | D | 56.3 |
| | F-BF + Urea | 6.10 | E | 50.4 |
| | F-BF + Calcium Nitrate | 4.66 | F | 42.5 |
| | O-BF | 3.27 | G | 10.2 |
| | O-Check (No N) | 2.25 | H | 0.0 |
| | F-BF | 1.37 | I | 3.1 |
| | F-Check (No N) | 1.06 | I | 0.0 |
| Lakeland | O-Calcium Nitrate + Urea | 8.54 | A | 60.4 |
| | F-Calcium Nitrate + Urea | 6.99 | B | 67.9 |
| | O-BF + Calcium Nitrate | 6.73 | BC | 39.1 |
| | O-Urea | 6.07 | CD | 26.5 |
| | F-Urea | 5.84 | D | 46.0 |
| | O-BF + Urea | 5.40 | DE | 19.8 |
| | F-BF + Urea | 5.09 | EF | 38.5 |
| | F-BF + Calcium Nitrate | 4.67 | F | 40.5 |
| | O-BF | 3.58 | G | 1.6 |
| | O-Check (No N) | 3.42 | G | 0.0 |
| | F-BF | 1.60 | H | 3.6 |
| | F-Check (No N) | 1.24 | H | 0.0 |

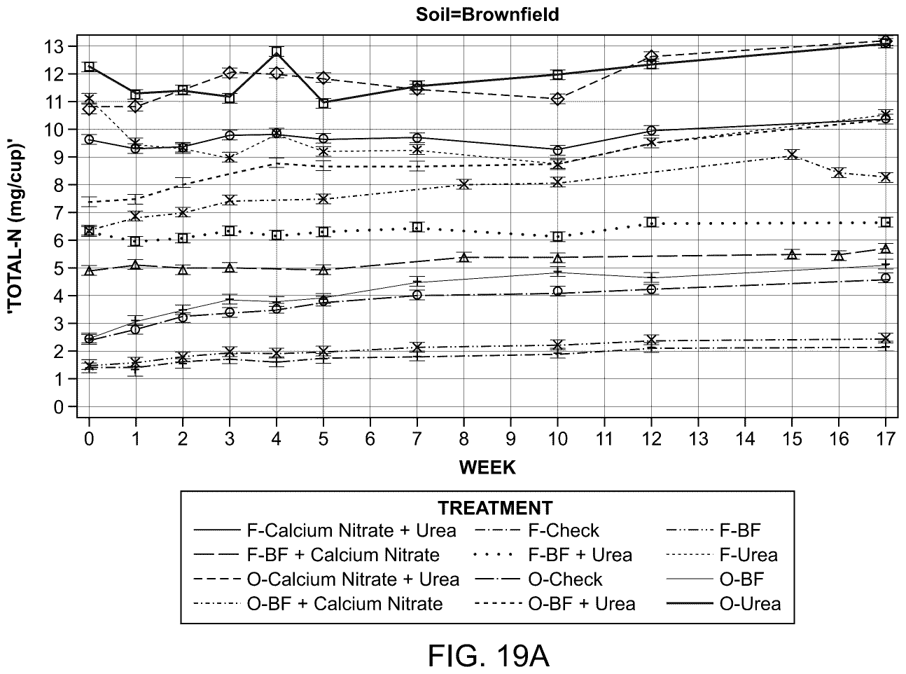

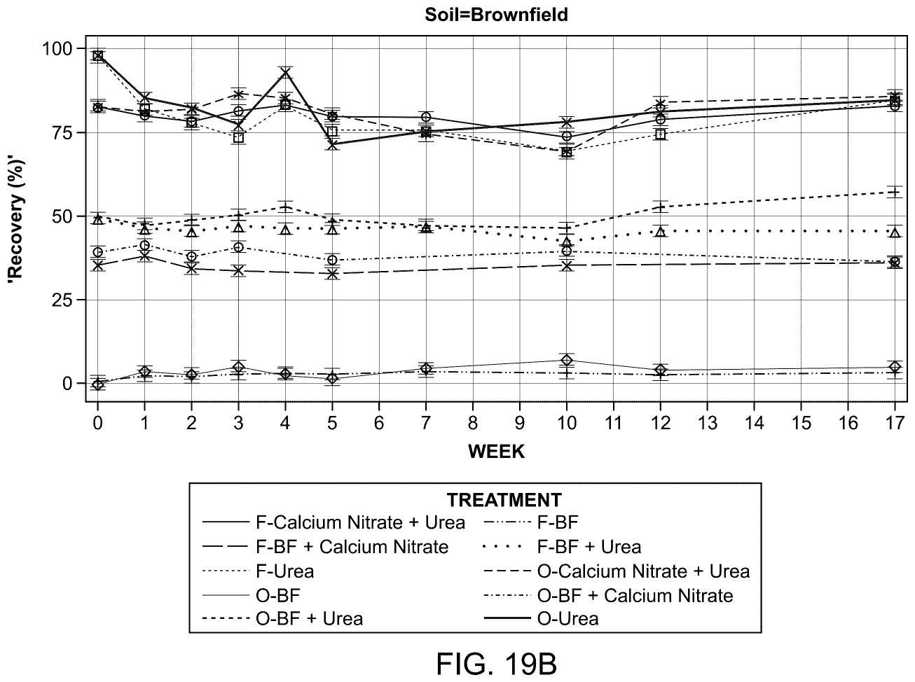

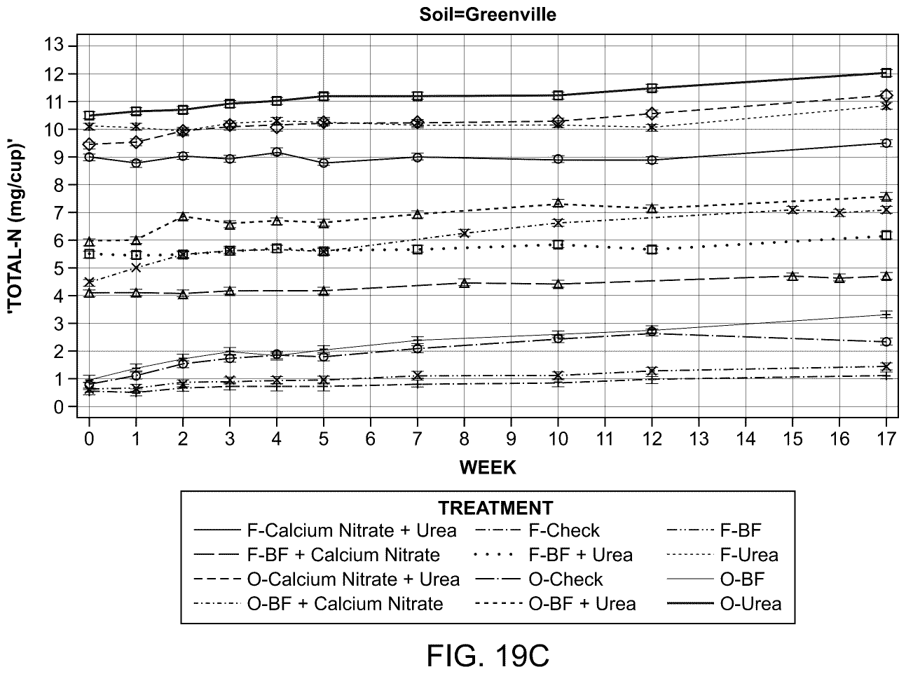

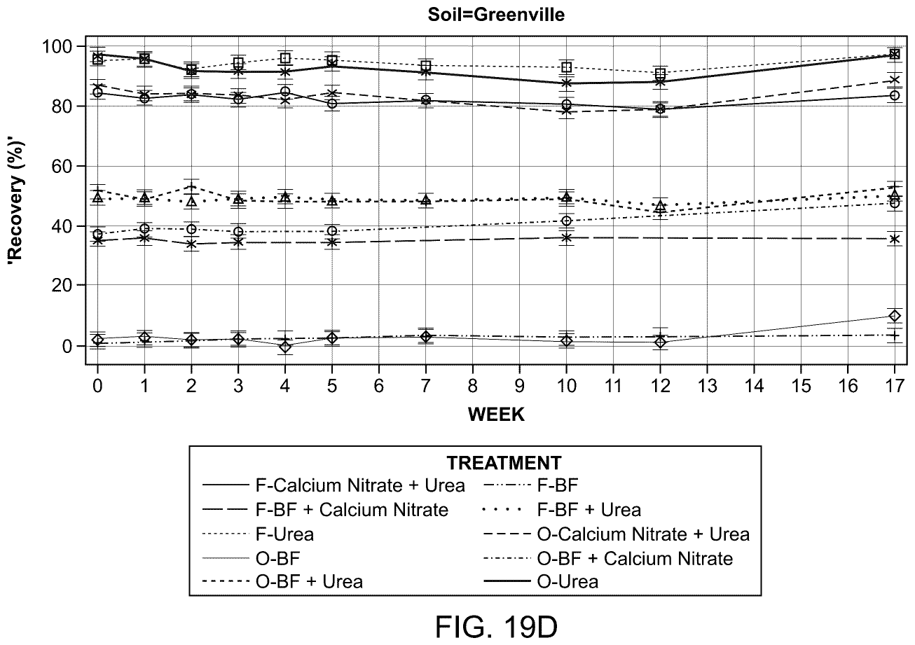

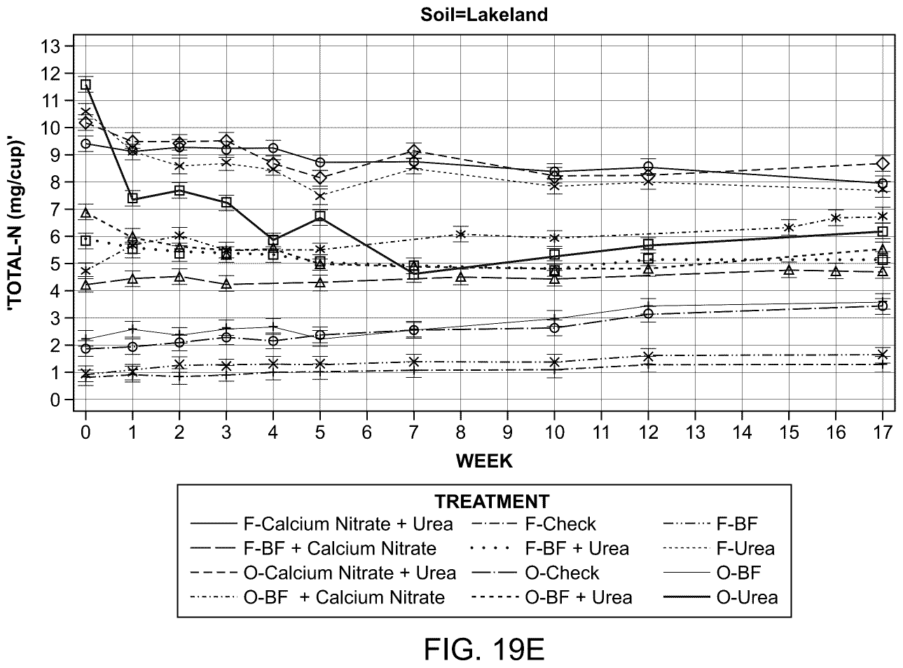

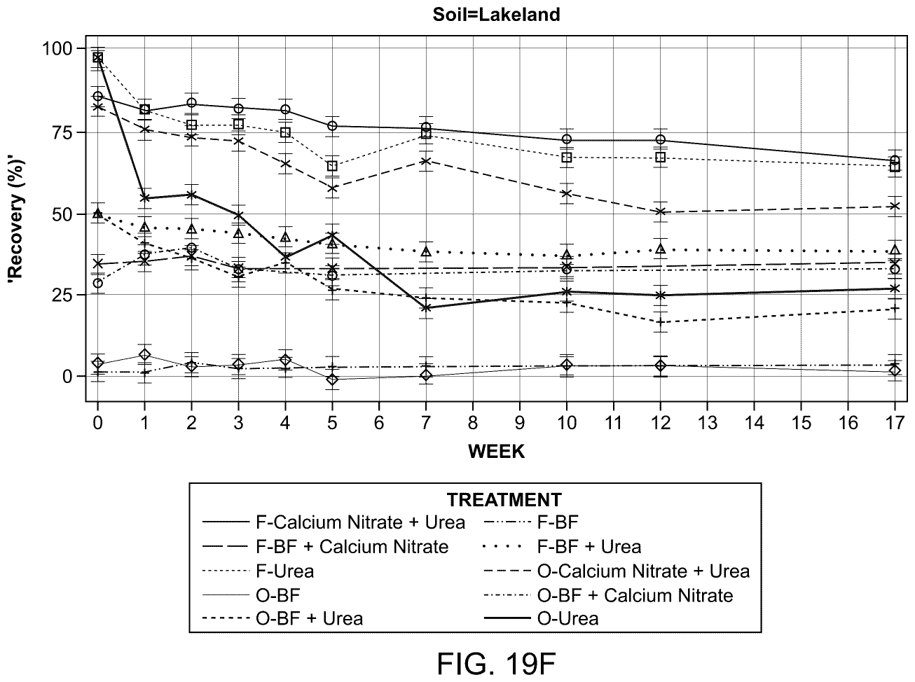

In all soil N transformation, analysis of variance showed that N products and amendments (Treatment), time (Week) and their interaction were significantly different (as expected), and this was also true for total available N (Table 23). Overall, total available N (urea-N+ammonium-N+nitrate-N) remained similar throughout the incubation period on Brownfield and Greenville soils, with likely losses due to ammonia volatilization loss and gains due to soil organic matter mineralization (FIGS. 19A-19D). However, on Lakeland sand, N treatments with urea had significantly lower total available soil N content and recoveries on week 17 than at zero-time (FIG. 19E-19F). Lakeland sand, with poor buffering capacity, was prone to higher ammonia volatilization loss.

In all soils, the greatest contribution towards total available soil N was from nitrate-N (Tables 22 and 24). Application of BF in organically amended Brownfield and Greenville soils, led to significant increases in available soil N content. Application of BF contributed 5% and 10% towards total N recoveries on organically amended Brownfield and Greenville soils, respectively (Table 24). In all soils with organic manure or fertilizer amendments, the application of BF alone or with 50% substitution of urea/calcium nitrate, resulted in higher soil available N; however, these increases were not always significantly higher. Also, in all soils when 50% of urea was replaced by BF (urea vs BF+urea comparison), the decrease in soil N content was less than 50% (Table 24). These results provided evidence that BF, even in the absence of plants, led to increases in available soil N content.

TABLE 23
Analysis of Variance for Total Available N.

| | | | Brownfield Soil | | Greenville Soil | | Lakeland Soil | |
|---|---|---|---|---|---|---|---|---|
| | Num | Den | F | Pr > | F | Pr > | F | Pr > |
| Effect | DF | DF | Value | F | Value | F | Value | F |
| WEEK | 12 | 239 | 669.50 | <.0001 | 432.89 | <.0001 | 224.86 | <.0001 |
| Treatment | 11 | 239 | 3522.27 | <.0001 | 460.37 | <.0001 | 293.79 | <.0001 |
| WEEK* | 96 | 239 | 44.86 | <.0001 | 28.60 | <.0001 | 6.48 | <.0001 |
| Treatment | | | | | | | | |

TABLE 24
Comparison of Total Available N and Total N Recovery for All Treatments
on Organically Amended (O) and Fertilized (F) Brownfield, Greenville,
and Lakeland Soils. (Differences are not significant for means
followed by the same letter at 5% level).

| SOIL | | Mean (mg N) | | Total N Recovery (%) |
|---|---|---|---|---|
| Brownfield | O-Calcium Nitrate + Urea | 13.22 | A | 100.3 |
| | O-Urea | 13.11 | A | 84.7 |
| | F-Urea | 10.56 | B | 84.5 |
| | F-Calcium Nitrate + Urea | 10.37 | B | 97.5 |
| | O-BF + Urea | 10.35 | B | 57.1 |
| | O-BF + Calcium Nitrate | 8.27 | C | 42.9 |
| | F-BF + Urea | 6.64 | D | 45.3 |
| | F-BF + Calcium Nitrate | 5.70 | E | 42.4 |
| | O-BF | 5.14 | F | 5.0 |
| | O-Check (No N) | 4.64 | G | 0.0 |
| | F-BF | 2.41 | H | 3.0 |
| | F-Check (No N) | 2.11 | H | 0.0 |
| Greenville | O-Urea | 12.01 | A | 97.0 |
| | O-Calcium Nitrate + Urea | 11.20 | B | 104.6 |
| | F-Urea | 10.82 | C | 97.2 |
| | F-Calcium Nitrate + Urea | 9.46 | D | 98.7 |
| | O-BF + Urea | 7.56 | E | 52.5 |
| | O-BF + Calcium Nitrate | 7.05 | F | 56.0 |
| | F-BF + Urea | 6.15 | G | 50.5 |
| | F-BF + Calcium Nitrate | 4.68 | H | 42.3 |
| | O-BF | 3.30 | I | 9.9 |
| | O-Check (No N) | 2.31 | J | 0.0 |
| | F-BF | 1.43 | K | 3.3 |
| | F-Check (No N) | 1.10 | L | 0.0 |
| Lakeland | O-Calcium Nitrate + Urea | 8.69 | A | 61.7 |
| | F-Calcium Nitrate + Urea | 7.92 | B | 78.4 |
| | F-Urea | 7.72 | B | 64.4 |
| | O-BF + Calcium Nitrate | 6.78 | C | 39.2 |
| | O-Urea | 6.19 | CD | 27.3 |
| | O-BF + Urea | 5.54 | DE | 20.8 |
| | F-BF + Urea | 5.17 | EF | 38.9 |
| | F-BF + Calcium Nitrate | 4.75 | F | 41.0 |
| | O-BF | 3.63 | G | 1.7 |
| | O-Check (No N) | 3.46 | G | 0.0 |
| | F-BF | 1.64 | H | 3.6 |
| | F-Check (No N) | 1.28 | H | 0.0 |

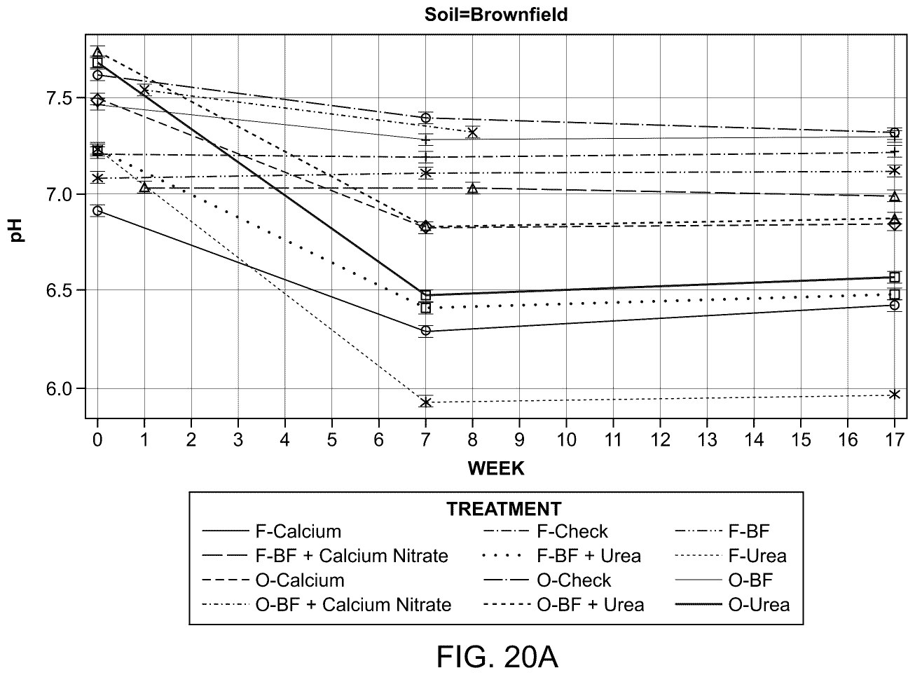

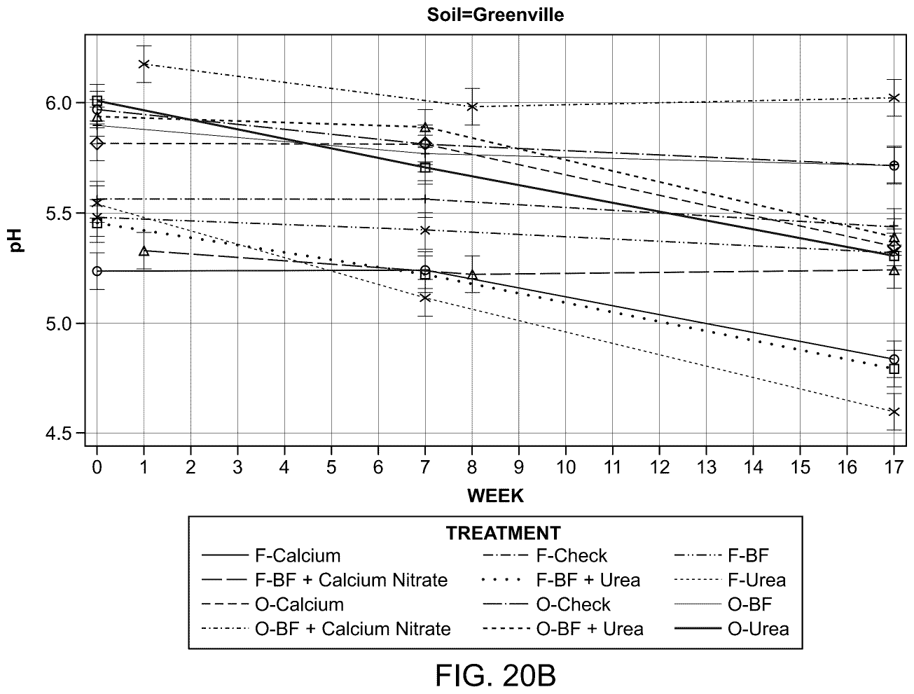

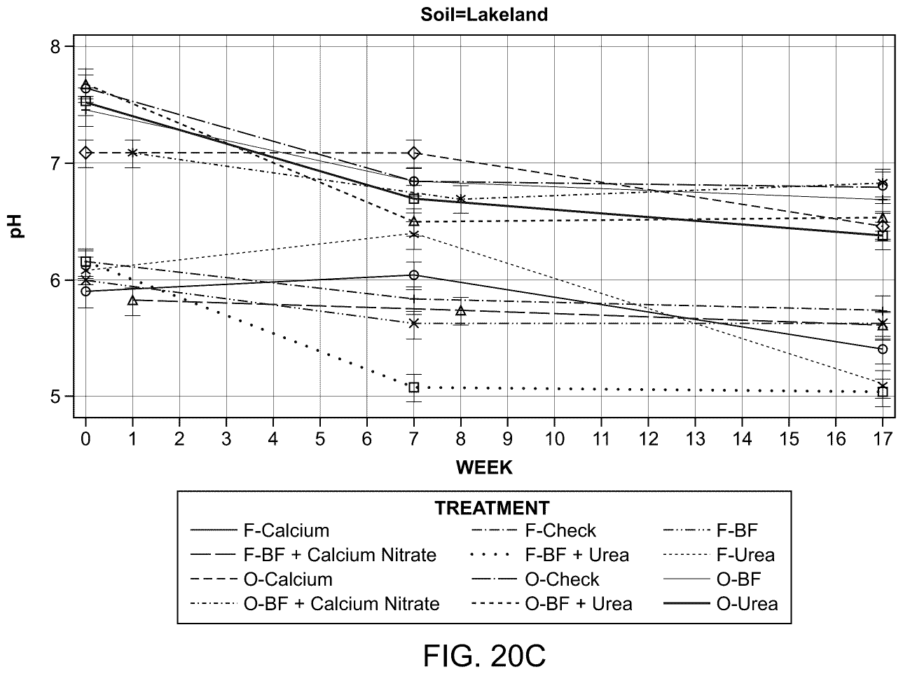

As with soil N transformation, analysis of variance for soil pH showed that N products and amendments-organic versus fertilized (treatment), time (week) and their interaction were significantly different (Table 25). On all soils and with organic manure or fertilizer application, all N products generally led to decline in soil pH (Figures FIGS. 20A-20C). Major decline in soil pH was associated with application of urea. The least changes in soil pH was observed with BF and BF+Calcium Nitrate treatments in all soils (Table 26). Hence, 50% replacement of urea with BF resulted in less soil acidification, with 0.25-0.53 units less pH decline on Brownfield soil and 0.16-0.29 units less pH decline on Greenville soil. Immediately after BF application (zero-time), soil pH was lower than Check (with no N application) on all soils and independent of organic manure or fertilizer amendments (Table 26).

TABLE 25
Analysis of Variance for Soil pH

| | | | Brownfield Soil | | Greenville Soil | | Lakeland Soil | |
|---|---|---|---|---|---|---|---|---|
| | Num | Den | F | Pr > | F | Pr > | F | Pr > |
| Effect | DF | DF | Value | F | Value | F | Value | F |
| WEEK | 4 | 70 | 648.89 | <.0001 | 39.49 | <.0001 | 58.67 | <.0001 |
| Treatment | 11 | 70 | 367.43 | <.0001 | 47.68 | <.0001 | 90.60 | <.0001 |
| WEEK* | 19 | 70 | 81.54 | <.0001 | 2.99 | 0.0004 | 6.02 | <.0001 |
| Treatment | | | | | | | | |

TABLE 26
Comparison of Soil pH for All Treatments on Organically Amended (O)
and Fertilized (F) Brownfield, Greenville, and Lakeland Soils.

| | Amendments and N | Mean pH | | | |
|---|---|---|---|---|---|
| SOIL | Products | Week 0 | Week 7 | Week 17 | Change |
| Brownfield | F-Urea | 7.23 | 5.92 | 5.96 | −1.28 |
| | O-Urea | 7.69 | 6.48 | 6.57 | −1.12 |
| | O-BF + Urea | 7.74 | 6.82 | 6.87 | −0.87 |
| | F-BF + Urea | 7.23 | 6.41 | 6.48 | −0.75 |
| | O-Calcium Nitrate + | 7.49 | 6.83 | 6.84 | −0.65 |
| | Urea | | | | |
| | F-Calcium Nitrate + | 6.91 | 6.29 | 6.42 | −0.49 |
| | Urea | | | | |
| | O-Check (No N) | 7.62 | 7.39 | 7.32 | −0.30 |
| | O-BF + Calcium Nitrate | 7.54 | 7.32 | 7.30 | −0.24 |
| | O-BF | 7.47 | 7.28 | 7.30 | −0.17 |
| | F-BF + Calcium Nitrate | 7.03 | 7.03 | 6.99 | −0.04 |
| | F-Check (No N) | 7.21 | 7.19 | 7.22 | 0.01 |
| | F-BF | 7.08 | 7.11 | 7.12 | 0.04 |
| Greenville | F-Urea | 5.54 | 5.12 | 4.60 | −0.95 |
| | O-Urea | 6.00 | 5.71 | 5.31 | −0.70 |
| | F-BF + Urea | 5.45 | 5.22 | 4.80 | −0.65 |
| | O-BF + Urea | 5.93 | 5.89 | 5.39 | −0.54 |
| | O-Calcium Nitrate + | 5.82 | 5.82 | 5.35 | −0.47 |
| | Urea | | | | |
| | F-Calcium Nitrate + | 5.24 | 5.24 | 4.84 | −0.40 |
| | Urea | | | | |
| | O-Check (No N) | 5.97 | 5.81 | 5.72 | −0.25 |
| | O-BF | 5.90 | 5.77 | 5.72 | −0.18 |
| | O-BF + Calcium Nitrate | 6.17 | 5.98 | 6.02 | −0.15 |
| | F-BF | 5.48 | 5.42 | 5.33 | −0.15 |
| | F-Check (No N) | 5.56 | 5.56 | 5.44 | −0.12 |
| | F-BF + Calcium Nitrate | 5.33 | 5.22 | 5.24 | −0.09 |
| Lakeland | O-Urea | 7.53 | 6.70 | 6.38 | −1.15 |
| | O-BF + Urea | 7.69 | 6.50 | 6.54 | −1.15 |
| | F-BF + Urea | 6.14 | 5.08 | 5.03 | −1.11 |
| | F-Urea | 6.07 | 6.39 | 5.11 | −0.97 |
| | O-Check (No N) | 7.64 | 6.84 | 6.80 | −0.84 |
| | O-BF | 7.44 | 6.85 | 6.68 | −0.76 |
| | O-Calcium Nitrate + | 7.08 | 7.07 | 6.45 | −0.63 |
| | Urea | | | | |
| | F-Calcium Nitrate + | 5.89 | 6.04 | 5.41 | −0.49 |
| | Urea | | | | |
| | F-Check (No N) | 6.12 | 5.83 | 5.74 | −0.38 |
| | F-BF | 5.99 | 5.62 | 5.63 | −0.36 |
| | O-BF + Calcium Nitrate | 7.09 | 6.69 | 6.83 | −0.26 |
| | F-BF + Calcium Nitrate | 5.82 | 5.74 | 5.61 | −0.21 |

### Example 11. Nitrogen Fixation Study

### Biofertilizer Alone

The Biofertilizer of the present disclosure (BF) was analyzed for its nitrogen fixation potential. Table 27 shows application rate of media (sterilized coconut coir (CC)), BF CFUs, and N fertilizer. Unlike earlier trials, no primer was added. Water holding capacity was measured following standard procedures (Smercina et al., 445 Plant and Soil 595-611 (2019)) upon receipt of the growth media. N fertilizer was provided as YaraLiva calcium nitrate and used to prepare a 0.8 mg mL-1 solution. BF was received as a 500 mL aliquot at 1×109 CFU mL-1. Upon receipt, BF was stored at 4° C. until further analysis. For this trial, CC media was autoclave sterilized as follows prior to additions and incubation. CC was placed in an autoclave safe container, autoclaved for 60 minutes on a gravity cycle using standard temperature, pressure, and drying times, then stored sealed at room temperature for 24 hours (Berns et al., 59 European journal of soil science 540-550 (2008)). After 24 hours, autoclave sterilization was repeated and then a subset of the sterilized CC was placed in liquid LB media to confirm sterilization (Berns et al., European Journal of Soil Science 59:540-550 (2008)). No growth was observed in LB after 48 hours from the sterilized CC indicating sufficient sterilization. It is important to note that autoclaving may not perfectly sterilize growth media and that many microorganisms are not currently culturable and therefore would not be observed in our LB culture (Nowak et al., 142 Zentralblatt für Mikrobiologie 521-525 (1987); Steen et al., 13 ISME Journal 3126-3130 (2009)). Along with testing BF addition rate, this study also tested impact of growth period on BF viability. Therefore, all treatments were replicated for a 7-day and a 14-day growth period.

TABLE 27
Growth Media, BF source, and Treatment conditions

| | | | | | Growth Period |
|---|---|---|---|---|---|
| Media | BF | Treatment | BF (CFU) | N fertilizer (%) | (days) |
| Sterilized CC | None | 0:0 | 0 | 0 | 7, 14 |
| Sterilized CC | None | 0:20 | 0 | 20 | 7, 14 |
| Sterilized CC | Standard | le7 | 1 × 10⁷ | 0 | 7, 14 |
| Sterilized CC | Standard | le8 | 1 × 10⁸ | 0 | 7, 14 |
| Sterilized CC | Standard | le9 | 1 × 10⁹ | 0 | 7, 14 |
| Sterilized CC | Standard | le10 | 1 × 10¹⁰ | 0 | 7, 14 |

BNF potential of BF was measured using the ¹⁵N₂ incorporation method (Smercina et al 2019). This method measures abundance of the stable isotope ¹⁵N, incorporated from ¹⁵N₂ gas, as a direct measure of BNF rates. A detailed step-by-step protocol used for these analyses is provided in the appendix below. Briefly, growth media was weighed into six replicate 20 mL incubation vials for each treatment (Table 27). CC media received N fertilizer and BF as pre-determined by the sponsor and in accordance with the appropriate experimental treatment (Table 27, Table 28). BF was prepared to specific CFU addition rates. CC media was then adjusted to 65% water holding capacity with sterile nanopure water (WHC; Table 28). Samples in the 14-day growth period treatment were then loosely covered with foil and incubated at room temperature before proceeding with the remainder of the incubation protocol. ¹⁵N₂ incubations for the 7-day growth period samples started immediately following WHC adjustment. At ¹⁵N₂ incubation start, vials were sealed and evacuated to remove ambient atmosphere. Vial atmospheres were then engineered with 1 ml of ¹⁵N₂, 10% O₂ by headspace volume and then balanced with ultra-high purity (UHP) Helium. All vials were incubated for 7 days in the dark at room temperature. Vials were then uncapped and dried at 105° C. for 48 hours before grinding to a fine powder. After grinding, 5-10 mg of each sample were tinned for IRMS analysis following standard procedures. Reference samples from previous trials were used as the baseline ¹⁵N abundance in the sample to calculate BNF rates as described below.

TABLE 28
BF N fertilizer, and water additions for each treatment.
Additions were identical for the two growth periods.

| | | | | | Volume N | |
|---|---|---|---|---|---|---|
| | | Mass of | Volume to | Volume BF | fertilizer | Volume |
| Media | Treatment | media (g) | 65% WHC | (μL) | (μL) | water (mL) |
| CC | 0:0 | 0.88 | 6 | 0 | 0 | 6 |
| | 0:20 | 0.88 | 6 | 0 | 100 | 5.9 |
| | 1e7 | 0.88 | 6 | 100 | 0 | 5.9 |
| | 1e8 | 0.88 | 6 | 100 | 0 | 5.9 |
| | 1e9 | 0.88 | 6 | 100 | 0 | 5.9 |
| | 1e10 | 0.88 | 6 | 100 | 0 | 5.9 |

BNF rates are determined based on net changes in ¹⁵N abundance of CC media between treatment and reference vials (Smercina et al., 445 Plant and Soil 595-611 (2019)). BNF rates were calculated as follows:

$$\frac{{AE}_{i} x {TN}_{i}}{{AE}_{atm} x t}$$

Where AEᵢ is the atom percent access measured in reference vial, TNᵢ is the total nitrogen in the treatment vial, AEₐₜₘ is the atom percent excess of the treatment vial atmosphere, and t is the incubation time in days (Smercina et al., 445 Plant and Soil 595-611 (2019)). BNF rates were calculated for all treatment vials. A single outlier in the 1e10 treatment for the 7-day growth period was removed prior to statistical analysis as this rate was found to be 5× greater than all other values in that treatment. Results were analyzed in the R stats package using a factorial ANOVA with BF addition rate, growth period, and their interactions as main effects followed by Tukey's HSD test.

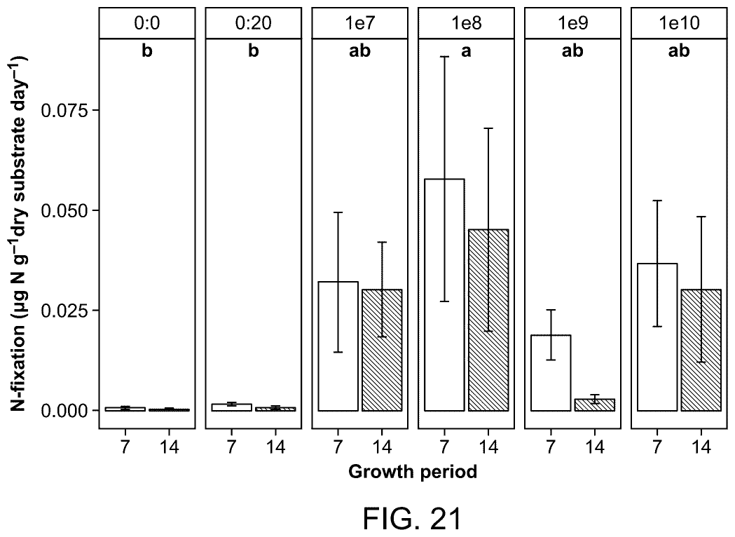

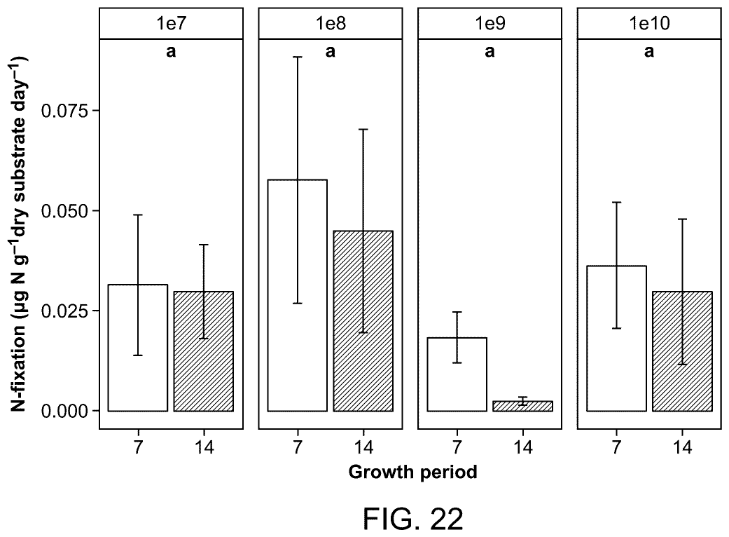

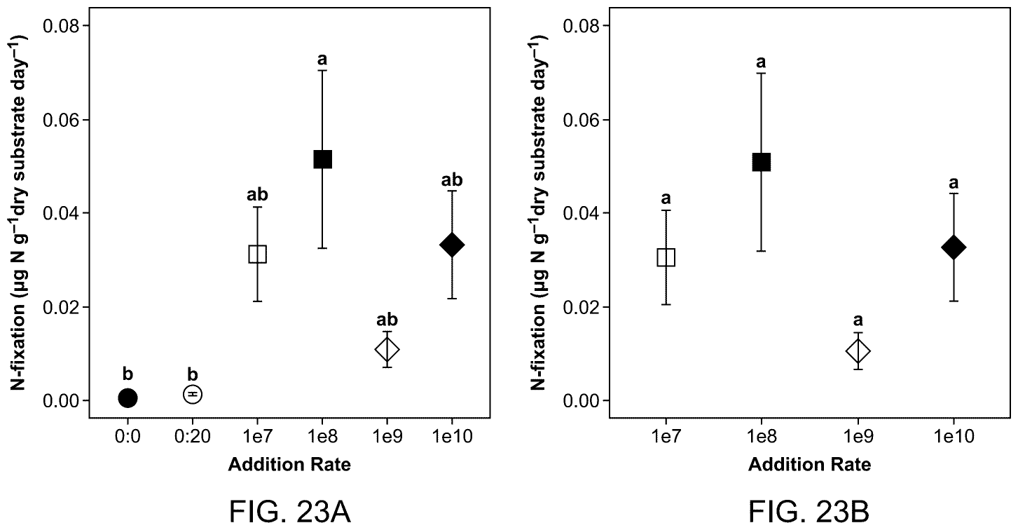

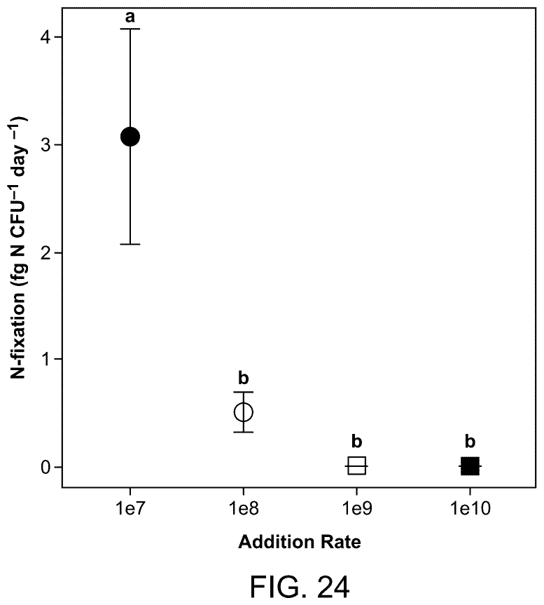

Potential BNF rates differed significantly by treatment (F=3.881, p=0.0042), but not by growth period (F=0.492, p=0.486) or their interaction (F=0.102, p=0.991). These differences were generally between samples with added BF versus no BF controls (FIG. 21; Table 29). No significant differences were observed between addition rate treatments. This was particularly evident in background corrected BNF rates (FIG. 22) where no significant differences were observed by treatment (F=1.697, p=0.183), growth period (F=0.508, p=0.480), or their interaction (F=0.060, p=0.980). Because there were no significant differences with growth period or growth period* addition rate interaction, values are also presented as average rates across growth period for greater visual clarity (FIG. 23). Lastly, BNF rates were calculated per CFU to estimate potential N contributions per cell for each addition rate treatment (FIG. 24). BNF per CFU differed significantly by addition rate (F=7.259, p<0.001), but not by growth period (F=0.024, p=0.879) or their interaction (F=0.007, p=0.999). Overall, the 1e7 treatment showed the greatest N-fixation per CFU with rates ˜5× greater than the next highest rate.

TABLE 29
Table of BNF rates presented as BNF per day, total BNF over incubation, and
BNF per CFU. Values are average BNF ± standard error (n = 6 per treatment).

| | | BNF (μg N g⁻¹ | Total BNF (μg N | Cellular BNF (fg |
|---|---|---|---|---|
| Treatment | Growth Period | substrate day⁻¹) | g⁻¹ substrate) | N CFU⁻¹ day⁻¹) |
| 0:0 | 7 | 0.0004 ± 0.0003 | 0.0031 ± 0.002 | |
| 0:0 | 14 | 0.0003 ± 0.0002 | 0.002 ± 0.002 | |
| 0:20 | 7 | 0.0018 ± 0.0004 | 0.012 ± 0.003 | |
| 0:20 | 14 | 0.0007 ± 0.0002 | 0.005 ± 0.002 | |
| le7 | 7 | 0.032 ± 0.017 | 0.224 ± 0.122 | 3.16 ± 1.74 |
| le7 | 14 | 0.030 ± 0.012 | 0.211 ± 0.082 | 2.97 ± 1.17 |
| le8 | 7 | 0.058 ± 0.031 | 0.405 ± 0.214 | 0.574 ± 0.306 |
| le8 | 14 | 0.045 ± 0.025 | 0.316 ± 0.177 | 0.447 ± 0.253 |
| le9 | 7 | 0.019 ± 0.006 | 0.132 ± 0.044 | 0.018 ± 0.006 |
| le9 | 14 | 0.003 ± 0.001 | 0.020 ± 0.008 | 0.003 ± 0.001 |
| le10 | 7 | 0.037 ± 0.016 | 0.257 ± 0.110 | 0.0036 ± 0.0016 |
| le10 | 14 | 0.030 ± 0.018 | 0.212 ± 0.126 | 0.003 ± 0.002 |

The results of this trial add to previous evidence that BF is capable of fixing a measurable amount of atmospheric N when under controlled laboratory conditions. BNF was consistently measured in all BF addition treatments and was detectable above background BNF activity. Overall, there were no significant differences across the BF addition rates. However, there was a trend towards greater BNF with additions at 1×10⁸ CFU compared to the other addition rates, with an average N-fixation of 0.051 (±0.02) μg N g⁻¹ dry substrate day⁻¹. In comparison to previous trials, results of this experiment were slightly lower on average, but generally within range of previously reported values. This observation of relatively lower BNF may relate to differences in application rate between trials. In a previous trial, BF was applied at 80% of field application rate rather than a specific CFU rate. Additional information regarding the specific CFU of BF at the time of application would be necessary to more accurately compare these rates across trials. Overall, addition rate results from this trial suggest a potential for diminishing returns with increasing rates of BF addition beyond 10⁸ CFU. When BNF rates were compared on a per CFU basis, there were clear differences by addition rate. Specifically, N fixation was greatest per CFU in the 1×10⁷ CFU treatment compared to the other three addition rates, with an overall average of 3.07 fg N CFU⁻¹ day⁻¹. There were no differences between the other three addition rates. This suggests that individual BF CFUs may be more active at lower addition rates. This again supports the idea of diminishing returns with increased application. Taken together with the per g dry substrate rates, these findings point towards an optimal addition rate between 1×10⁷ and 1×10⁸ CFU for the conditions tested in this study (e.g. sterile media, controlled lab studies).

The second aspect of this trial was to evaluate the viability of BF and its potential N-fixation rates over time. Here, N-fixation was compared by BF over two growth periods, 7-day and 14-day. In both cases, incubation time with ¹⁵N₂ was carried out over 7 days. However, for the 14-day growth period, an additional 7-day pre-incubation of BF was performed. This approach allowed the assessment of change in N-fixation potential between the first week after application and the second week. Across all addition rates, no difference was found between the 7-day and 14-day N-fixation rates. This provides strong evidence that BNF potential by BF is consistently maintained, and that cells are viable up to 14 days after application to growth media in the controlled lab setting. Lastly, BNF rates across all treatments within this trial were measured under controlled conditions where BF is the dominant organism. This limits potential competition and interactions with other microorganisms or plants.

### Biofertilizer and Other Diazotrophic Bacteria in Sterile and Non-Sterile Media

Biological N-fixation (BNF) rates were measured for the biofertilizer (BF) of the present disclosure and four other diazotrophic bacteria in sterile and non-sterile coconut coir (CC) media.

Diazotroph cultures including Azotobacter vinelandii DJ, Azospirillum brasiliense Sp7, Paenibacillus polymyxa DSM 36, and Sphingomonas azotifigens DSM 18530 were prepared from culture stocks. N fertilizer, BF, and diazotroph cultures were added at rates in accordance with Table 30. No primer was added. Water holding capacity was measured following standard procedures (Smercina et al., 445 Plant and Soil, 595-611 (2019)) upon receipt of the growth media. N-fertilizer was provided as YaraLiva calcium nitrate and used to prepare a 0.8 mg mL⁻¹ solution. For this trial, sterilized and nonsterilized CC media were used. To sterilize the CC, media was placed in an autoclave-safe container, autoclaved for 60 minutes on a gravity cycle using standard temperature, pressure, and drying times, then stored sealed at room temperature for 24 hours (Berns et al., 59 European Journal of Soil Science, 540-550 (2008)). After 24 hours, autoclave sterilization was repeated.

TABLE 30
growth media, organism, and treatment conditions

| Media | Organism | Treatment | CFU | N fertilizer (%) |
|---|---|---|---|---|
| Non-sterile CC | None | 0:0 | 0 | 0 |
| Non-sterile CC | None | 0:20 | 0 | 20 |
| Non-sterile CC | A. brasilense | NS AB | 1 × 10⁸ | 0 |
| Non-sterile CC | A. vinelandii | NS AV | 1 × 10⁸ | 0 |
| Non-sterile CC | P. polymyxa | NS PP | 1 × 10⁸ | 0 |
| Non-sterile CC | S. azotifigens | NS SA | 1 × 10⁸ | 0 |
| Non-sterile CC | BF | NS BF | 1 × 10⁸ | 0 |
| Sterilized CC | A. brasilense | S AB | 1 × 10⁸ | 0 |
| Sterilized CC | A. vinelandii | S AV | 1 × 10⁸ | 0 |
| Sterilized CC | P. polymyxa | S PP | 1 × 10⁸ | 0 |
| Sterilized CC | S. azotifigens | S SA | 1 × 10⁸ | 0 |
| Sterilized CC | BF | S BF | 1 × 10⁸ | 0 |

BNF potential of BF and diazotroph cultures was measured using the ¹⁵N₂ incorporation method (Smercina et al., 445 Plant and Soil 595-611 (2019)). This method measures abundance of the stable isotope ¹⁵N, incorporated from ¹⁵N₂ gas, as a direct measure of BNF rates. Briefly, growth media was weighed into six replicate 20 mL incubation vials for each treatment (Table 30). CC media received N fertilizer, BF, or diazotroph cultures in accordance with the appropriate experimental treatment (Table 30, Table 31). BF and diazotroph cultures were prepared to an addition rate of 1×10⁸ CFU. CC media was then adjusted to 65% water holding capacity with sterile nanopure water (WHC; Table 31). At ¹⁵N₂ incubation start, vials were sealed and evacuated to remove ambient atmosphere. Vial atmospheres were then engineered with 1 mL of ¹⁵N₂, 10% O₂ by headspace volume and then balanced with ultra-high purity (UHP) Helium. All vials were incubated for 7 days in the dark at room temperature. Vials were then uncapped and dried at 105° C. for 48 hours before grinding to a fine powder. After grinding, 5-10 mg of each sample were tinned for IRMS analysis following standard procedures. Reference samples were used as the baseline 15N abundance in the sample to calculate BNF rates as described below.

TABLE 31
BF, N fertilizer, and water additions for each treatment.
Additions were identical for the two growth periods.

| | | | | Volume | | |
|---|---|---|---|---|---|---|
| | | Mass of | Volume to 65% | bacteria | Volume N | Volume |
| Media | Treatment | media (g) | WHC (mL) | (μL) | fertilizer (μL) | water (mL) |
| Sterile and | 0:0 | 0.88 | 6 | 0 | 0 | 6 |
| non-sterile | 0:20 | 0.88 | 6 | 0 | 100 | 5.9 |
| Coconut | A. brasilense | 0.88 | 6 | 100 | 0 | 5.9 |
| Coir | A. vinelandii | 0.88 | 6 | 100 | 0 | 5.9 |
| | P. polymyxa | 0.88 | 6 | 100 | 0 | 5.9 |
| | S. azotifigens | 0.88 | 6 | 100 | 0 | 5.9 |
| | BF | 0.88 | 6 | 100 | 0 | 5.9 |

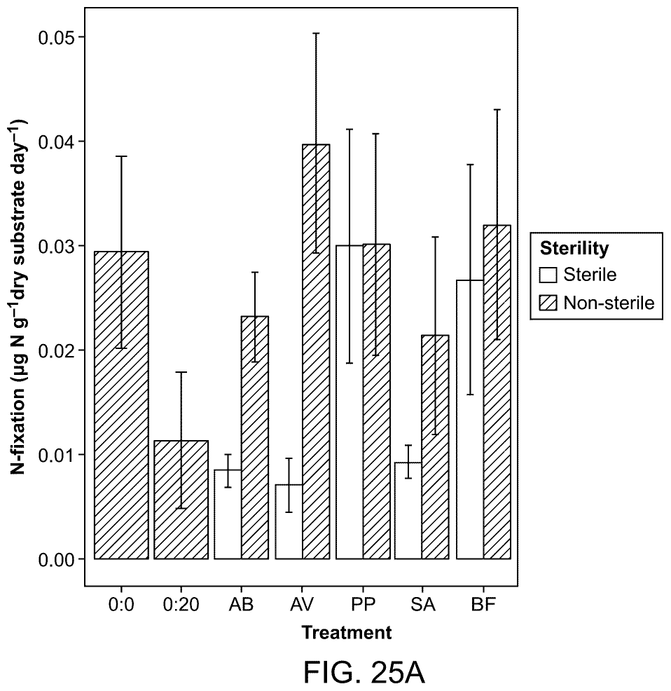

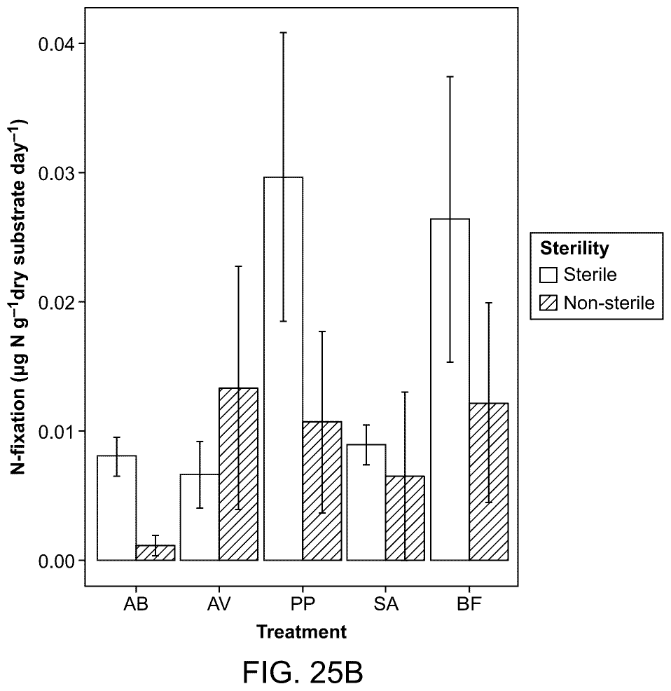

No significant differences were found in potential BNF rates between organisms (F=1.372, p=0.241; FIG. 25A). Potential BNF differed significantly by CC sterilization (F=6.024, p=0.017), but the interaction between organism and CC sterilization was not significant (F=1.107, 0.362) suggesting differences by CC sterilization alone were driven by the presence of background controls in this statistical analysis. Background corrected values are a more accurate representation of BNF rates resulting from BF or diazotroph activity (FIG. 25B). No significant differences were observed in the background corrected BNF rates by organism (F=1.965, p=0.114), CC sterilization (F=2.556, p=0.116), or their interaction (F=1.003, p=0.415). Though not statistically significant, BNF rates of BF were generally at the upper end of those measured in association with the other four diazotrophic species, regardless of the CC sterilization treatment. Additionally, BNF rates measured for BF and other diazotrophs in this trial are within range of those measured in FIGS. 22-24 of the equivalent addition rate (1×10⁸ CFU) where the BF rate from FIGS. 22-24 was not significantly different from rates measured in this trial (F=1.938, p=0.117).

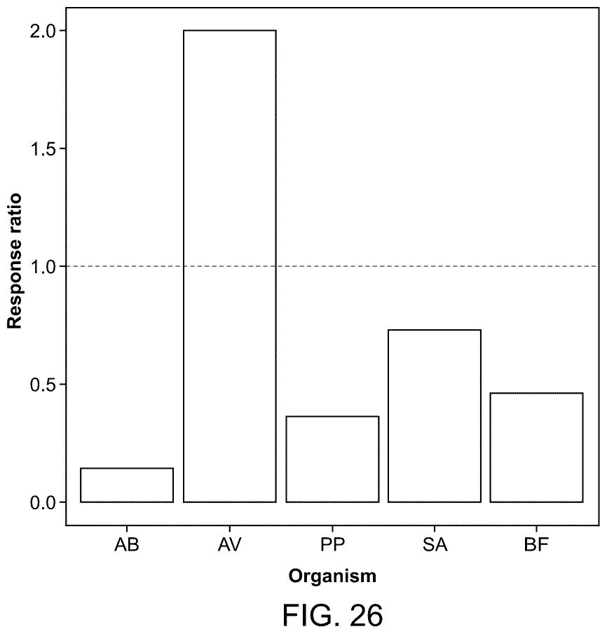

Lastly, to understand the impact that the presence of a native microbial community has on BNF rates, response ratios of BNF activity in non-sterile CC compared to sterile CC for each organism were calculated using background corrected BNF rates (FIG. 26). Response ratios were calculated to compare BNF rates in non-sterile vs. sterile growth conditions for each organism. These response ratios were calculated using the average background corrected BNF rates and determined by dividing non-sterile treatment rates by sterile treatment rates, as seen in Table 32. These ratios generally suggest that diazotrophic bacteria, including BF tend to fix less N when grown in association with a complex microbial community. Only, A. vinelandii (AV) treatments reported a response ratio greater than 1, indicating this organism performed better when in non-sterile vs. sterile conditions. These results confirmed the presence of an endemic diazotrophic community in non-sterile CC. The resulting BNF rates in 0:0 and 0:20 control samples must be accounted for through background subtraction to provide an accurate measure of BNF from the organism of interest (e.g., BF). Accounting for this background BNF, these results provided additional evidence of BNF activity of BF and demonstrate BF likely is capable of fixing N at a greater rate than native soil diazotrophs, such as those included in this study. However, BNF by BF appears to be suppressed by the presence of other microorganisms as indicated by the relatively lower BNF rates when grown in non-sterile media relative to sterile media. Three of the other diazotrophs included in this study also showed reduced capacity for BNF when in the presence of the microbial community from non-sterile CC. Given the evidence for an endemic diazotrophic community in the non-sterile CC, this suggests competition between this endemic community and added diazotrophs (e.g., BF, AB, PP, and SA). Because BF has been specifically designed to come with its own carbon energy source, these findings suggest competition with the endemic diazotrophic community may be for other resources, such as the micronutrients needed to synthesize nitrogenase including molybdenum and iron.

TABLE 32
Table of BNF rates presented as BNF per day, background corrected
BNF per day, total BNF over incubation, and response ratios. Values
are average BNF ± standard error (n = 6 per treatment).

| | BNF | Background Corrected | Total BNF | |
|---|---|---|---|---|
| | (μg N g⁻¹ substrate | BNF | (μg N g⁻¹ | Response |
| Treatment | day⁻¹) | (μg N g⁻¹ substrate day⁻¹) | substrate) | Ratio |
| 0:0 Non-sterile | 0.029 ± 0.009 | — | — | — |
| 0:20 Non-sterile | 0.011 ± 0.007 | — | — | — |
| AB Sterile | 0.008 ± 0.002 | 0.008 ± 0.002 | 0.056 ± 0.011 | 0.14 |
| AB Non-sterile | 0.023 ± 0.004 | 0.001 ± 0.001 | 0.008 ± 0.005 | 0.14 |
| AV Sterile | 0.007 ± 0.003 | 0.007 ± 0.003 | 0.047 ± 0.002 | 1.99 |
| AV Non-sterile | 0.040 ± 0.011 | 0.013 ± 0.001 | 0.093 ± 0.066 | 1.99 |
| PP Sterile | 0.030 ± 0.011 | 0.030 ± 0.011 | 0.207 ± 0.078 | 0.36 |
| PP Non-sterile | 0.030 ± 0.011 | 0.011 ± 0.0001 | 0.075 ± 0.049 | 0.36 |
| SA Sterile | 0.009 ± 0.002 | 0.009 ± 0.002 | 0.062 ± 0.011 | 0.73 |
| SA Non-sterile | 0.021 ± 0.009 | 0.007 ± 0.007 | 0.046 ± 0.046 | 0.73 |
| BF Sterile | 0.027 ± 0.011 | 0.027 ± 0.011 | 0.185 ± 0.077 | 0.46 |
| BF Non-sterile | 0.032 ± 0.011 | 0.012 ± 0.008 | 0.085 ± 0.054 | 0.46 |

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A growth medium for a nitrogen-fixing microorganism comprising a compound nutrient source, a salt, a buffer, a carbon source, and a metal source,
wherein the compound nutrient source comprises two or more constituent components,
wherein the salt comprises a soluble salt of the constituent component,
wherein the compound nutrient source has a solubility lower than the solubility of the soluble salt of the constituent component, and
wherein the concentration of the constituent component salt is below the toxicity level of the nitrogen-fixing microorganism.

2. The medium of claim 1, wherein the compound nutrient source comprises ammonium (NH₄⁺), magnesium (Mg²⁺), and phosphate (H₂PO₄⁻, HPO₄²⁻, PO₄³⁻).

3. The medium of claim 1 or 2, wherein the compound nutrient source is selected from ammonium magnesium phosphate (MgNH₄PO₄), a hydrate thereof.

4. The medium of claim 3, wherein the compound nutrient source is ammonium magnesium phosphate hexahydrate (MgNH₄PO₄·6H₂O).

5. The medium of any one of claims 1-4, wherein the constituent component salt is selected from a soluble salt of ammonium (NH₄⁺), magnesium (Mg²⁺), or phosphate (H₂PO₄⁻, HPO₄²⁻, PO₄³⁻), or a combination thereof.

6. The medium of any one of claims 1-5, wherein the medium comprises one of each of the soluble salts of ammonium (NH₄⁺), magnesium (Mg²⁺), and phosphate (H₂PO₄⁻, HPO₄²⁻, PO₄³⁻).

7. The medium of claim 6, wherein the medium comprises potassium phosphate monobasic (K₂HPO₄), potassium phosphate dibasic (KH₂PO₄), magnesium sulfate heptahydrate (MgSO₄·7H₂O), or ammonium sulfate ((NH₄)₂SO₄), or a combination thereof.

8. The medium of any one of claims 1-7, wherein the concentration of ammonium is less than about 20 mM.

9. The medium of claim 8, wherein the concentration of ammonium is from about 5 mM to about 12 mM.

10. The medium of any one of claims 1-9, wherein the compound nutrient source further comprises metal carbonate.

11. The medium of claim 10, wherein the metal carbonate comprises a divalent cationic metal.

12. The medium of claim 11, wherein the divalent cationic metal comprises Ca²⁺, Mg²⁺, Fe²⁺, Ni²⁺, or Co²⁺, or a combination thereof.

13. The medium of claim 12, wherein the divalent cation metal is Fe²⁺.

14. The medium of claim 13, wherein the medium comprises ferric citrate and ammonium bicarbonate ((NH₄)HCO₃).

15. The medium of any one of claims 1-14 further comprising a soluble salt of calcium (Ca²⁺).

16. The medium of claim 15, wherein the salt is CaSO₄.

17. The medium of any one of claims 1-16, wherein the carbon source is selected from an autotrophic carbon source, or a heterotrophic carbon source, or a combination thereof.

18. The medium of claim 17, wherein the carbon source is an autotrophic carbon source.

19. The medium of claim 18, wherein the autotrophic carbon source is methanol.

20. The medium of any one of claims 1-19, wherein the metal source comprises a trace metal or semi-metal source.

21. The medium of claim 20, wherein the trace metal or semi-metal source is ferric citrate, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), or nickel (II) sulfate hexahydrate (NiSO₄·6H₂O), or a combination thereof.

22. The medium of any one of claims 1-21, wherein the medium further comprises a nitrogen-fixing microorganism.

23. The medium of claim 22, wherein the nitrogen-fixing microorganism expresses nitrogenase and accumulates a microbial intracellular storage compound (MISC).

24. The medium of claim 23, wherein the MISC comprises a polyhydroxyalkanoate (PHA), a polyphosphate (PolyP), or a lipid, or a combination thereof.

25. The medium of claim 24, wherein the MISC is a PHA.

26. The medium of claim 25, wherein the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof.

27. The medium of claim 26, wherein the PHA is PHB.

28. The medium of any one of claims 22-27, wherein the nitrogen-fixing microorganism comprises bacteria.

29. The medium of claim 28, wherein the nitrogen-fixing microorganism is a PHB-producing bacteria.

30. The medium of any one of claims 22-27, wherein the nitrogen-fixing microorganism comprises archea.

31. The medium of any one of claims 22-27, wherein the nitrogen-fixing microorganism comprises fungi.

32. The medium of any one of claims 28-31, wherein the nitrogen-fixing microorganism comprises one or more strains of Acidiphilium species, Acidiphilium multivorum, Alcaligenes species, Alcaligenes paradoxus, Arthrobacter species, Azohydromonas species, Azohydromonas australica, Azohydromonas species, Azohydromonas lata, Azospirillum species, Azospirillum amazonsense, Azospirillum lipoferum, Azospirillum thiophilum, Azotobacter vinelandii, Beggiatoa species, Beggiatoa alba, Beijerinckia species, Beijerinckia mobilis, Bradyrhizobium species, Bradyrhizobium elnakii, Bradyrhizobium japonicum, Bradyrhizobium japonicum (strain USDA 122), Burkholderia species, Burkholderia vietnameiensis, Cupriavidus species, Cupriavidus necator, Derxia species, Derxia gummosa, Herbaspirillum species, Herbaspirillum autrotrophicum, Hydrogenophaga species, Hydrogenophaga pseudoflava, Mesorhizobium species, Mesorhizobium alhagi, Methylibium species, Methylibium petroleiphilum, Methylocapsa species, Methylocapsa aurea, Methyloferula species, Methyloferula stellate, Methyloversatilis species, Methyloversatilis universalis, Microcyclus species, Microcyclus aquaticus, Microcyclus ebruneus, Nitrosococcus species, Nitrosococcus oceani, Nitrosomonas communis, Nitrospirillum amazonense, Nocardia species, Nocardia autotrophica, Nocardia opaca, Oligotropha species, Oligotropha carboxidovorans, Pannonibacter species, Pannonibacter phragmitetus, Paracoccus species, Paracoccus denitrificans, Paracoccus pantrophus, Paracoccus yeei, Pelagibaca species, Pelagibaca bermudensis, Pseudomonas species, Pseudomonas facilis, Pseudooceanicola species, Pseudooceanicola atlanticus, Ralstonia species, Ralstonia eutropha, Renobacter species, Renobacter vacuolatum, Rhizobium species, Rhizobium gallicum, Rhizobium japonicum, Rhodobacter species, Rhodobacter capsulatus, Rhodobacter sphaeroides, Rhodomicrobium species, Rhodomicrobium vannielii, Rubrivivax species, Rubrivivax gelatinosus, Salipiger species, Salipiger mucosus, Sinorhizobium species, Sinorhizobium americanum, Sinorhizobium fredii, Sinorhizobium meliloti, Skermanella species, Skermanella stibiiresistens, Stappia species, Stappia aggregate, Thauera species, Thauera humireducens, Variovorax species, Variovorax paradoxus, Xanthobacter species, or Xanthobacter autotrophicus.

33. The medium of claim 32, wherein the nitrogen-fixing microorganism is Xanthobacter autotrophicus.

34. The medium of claim 32, wherein the nitrogen-fixing microorganism is Ralstonia eutropha.

35. The medium of any one of claims 1-34 comprising ammonium magnesium phosphate hexahydrate (MgNH₄PO₄·6H₂O), ferric citrate, ammonium bicarbonate ((NH₄)HCO₃), potassium phosphate monobasic (K₂HPO₄), potassium phosphate dibasic (KH₂PO₄), magnesium sulfate heptahydrate (MgSO₄·7H₂O), calcium sulfate dihydrate (CaSO₄·2H₂O), ammonium sulfate ((NH₄)₂SO₄), methanol, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O).

36. The medium of any one of claims 1-34 comprising ammonium dihydrogen phosphate ((NH₄)H₂PO₄), magnesium hydroxide (Mg(OH)₂), ferric citrate, ammonium bicarbonate ((NH₄)HCO₃), potassium phosphate monobasic (K₂HPO₄), potassium phosphate dibasic (KH₂PO₄), magnesium sulfate heptahydrate (MgSO₄·7H₂O), calcium sulfate dihydrate (CaSO₄·2H₂O), ammonium sulfate ((NH₄)₂SO₄), methanol, boric acid (H₃BO₃), manganese sulfate monohydrate (MnSO₄·H₂O), sodium molybdate dihydrate (Na₂MoO₂·2H₂O), zinc sulfate heptahydrate (ZnSO₄·7H₂O), copper (II) nitrate hemi-pentahydrate (Cu(NO₃)₂·2.5H₂O), and nickel (II) sulfate hexahydrate (NiSO₄·6H₂O).

37. A biofertilizer comprising the medium of any one of claims 22-36.

38. A method of enhancing the accumulation of a MISC in a nitrogen-fixing microorganism, comprising:
a) growing a culture of the nitrogen-fixing microorganism in a growth medium comprising a compound nutrient source, a salt, a buffer, a carbon source, and a metal source,
wherein the compound nutrient source comprises three or more constituent components,
wherein the salt comprises a soluble salt of the constituent component,
wherein the compound nutrient source has a solubility lower than the solubility of the soluble salt of the constituent component,
wherein the concentration of the constituent component salt is below the toxicity level of the nitrogen-fixing microorganism,
wherein the culture of the nitrogen-fixing microorganism is grown to a specified concentration measured by an optical density at 600 nm (OD₆₀₀), and
wherein the compound nutrient source is added in a single batch; and
b) adding a carbon source in an amount that exceeds the amount of sufficient for the growth of the microorganism to enhance the accumulation of the MISC in the microorganism.

39. The method of claim 38 further comprising inoculating the medium with an initial culture of the nitrogen-fixing microorganism.

40. The method of claim 39, wherein the medium is inoculated with the initial culture of the nitrogen fixing microorganism.

41. The method of claim 40, wherein the medium is inoculated with the initial culture of the nitrogen fixing microorganism having an OD₆₀₀ greater than about 2.

42. The method of any one of claims 38-41, wherein the culture of the microorganism has an OD₆₀₀ greater than about 0, greater than about 2, greater than about 5, greater than about 7, greater than about 10, greater than about 15, greater than about 20, greater than about 25, greater than about 30, greater than about 35, or greater than about 40.

43. The method of any one of claims 38-42, wherein the culture of the microorganism has an OD₆₀₀ of a range from about 0 to about 40.

44. The method of any one of claims 38-42, wherein the culture of the microorganism has an OD₆₀₀ from about 0 to about 1, from about 0 to about 2, from about 0 to about 3, from about 0 to about 4, from about 0 to about 5, from about 2 to about 3, from about 2 to about 4, from about 2 to about 5, from about 3 to about 4, from about 3 to about 5, from about 3 to about 7, from about 4 to about 7, from about 4 to about 8, from about 4 to about 10, from about 5 to about 10, from about 5 to about 12, from about 5 to about 15, from about 5 to about 20, from about 7 to about 10, from about 7 to about 12, from about 7 to about 15, from about 7 to about 20, from about 8 to about 10, from about 8 to about 12, from about 8 to about 15, from about 8 to about 20, from about 9 to about 10, from about 9 to about 12, from about 9 to about 15, from about 9 to about 20, from about 10 to about 12, from about 10 to about 15, from about 10 to about 20, from about 12 to about 15, from about 12 to about 20, from about 12 to about 23, from about 12 to about 25, from about 15 to about 20, from about 15 to about 23, from about 15 to about 25, from about 17 to about 20, from about 17 to about 23, from about 17 to about 25, from about 17 to about 27, from about 20 to about 23, from about 20 to about 25, from about 20 to about 27, from about 20 to about 30, from about 23 to about 25, from about 23 to about 27, from about 23 to about 30, from about 23 to about 33, from about 25 to about 27, from about 25 to about 30, from about 25 to about 33, from about 25 to about 35, from about 27 to about 30, from about 27 to about 33, from about 27 to about 35, from about 27 to about 37, from about 30 to about 33, from about 30 to about 35, from about 30 to about 37, from about 30 to about 40, from about 33 to about 35, from about 33 to about 37, from about 33 to about 40, from about 33 to about 43, from about 35 to about 37, from about 35 to about 40, from about 35 to about 43, from about 35 to about 45, from about 37 to about 40, from about 37 to about 43, from about 37 to about 45, from about 40 to about 43, from about 40 to about 45, or from about 43 to about 45.

45. The method of claim 44, wherein the culture of the microorganism has an OD₆₀₀ of about 0, about 2, about 3, about 4, about 5, about 7, about 8, about 9, about 10, about 12, about 15, about 20, about 23, about 25, about 27, about 30, about 33, about 35, about 37, about 40, about 43, or about 45.

46. The method of claim 42, wherein the culture of the microorganism has an OD₆₀₀ greater than about 2.

47. The method of claim 44, wherein the culture of the microorganism has an OD₆₀₀ from about 2 to about 4.

48. The method of claim 45, wherein the culture of the microorganism has an OD₆₀₀ of about 2.

49. The method of claim 42, wherein the culture of the microorganism has an OD₆₀₀ greater than about 20.

50. The method of claim 45, wherein the culture of the microorganism has an OD₆₀₀ of about 20.

51. The method of claim 42, wherein the culture of the microorganism has an OD₆₀₀ greater than about 25.

52. The method of claim 45, wherein the culture of the microorganism has an OD₆₀₀ of about 25.

53. The method of any one of claims 38-52, wherein the MISC is a PHA, a PolyP, or a lipid, or a combination thereof.

54. The method of claim 53, wherein the MISC is a PHA.

55. The method of claim 54, wherein the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof.

56. The method of claim 55, wherein the PHA is PHB.

57. The method of claim 38, wherein the accumulation of MISC is greater than about 10%, greater than about 12%, greater than about 15%, greater than about 20%, greater than about 25%, greater than about 30%, greater than about 35%, or greater than about 40%.

58. The method of claim 57, wherein the accumulation of MISC is greater than about 25% to about 40%.

59. The method of claim 58, wherein the accumulation of MISC is about 25%.

60. The method of claim 56, wherein the accumulation of MISC is from about 10% to about 12%, from about 10% to about 15%, from about 10% to about 20%, from about 10% to about 25%, from about 12% to about 15%, from about 12% to about 20%, from about 12% to about 25%, from about 12% to about 30%, from about 15% to about 20%, from about 15% to about 25%, from about 15% to about 30%, from about 15% to about 35%, from about 20% to about 25%, from about 20% to about 30%, from about 20% to about 35%, from about 20% to about 40%, from about 25% to about 30%, from about 25% to about 35%, from about 25% to about 40%, from about 30% to about 35%, from about 30% to about 40%, or from about 35% to about 40%.

61. The method of claim 56, wherein the accumulation of MISC is about 10%, about 12%, about 15%, about 20%, about 25%, about 30%, about 35%, or about 40%.

62. The method of claim 61, wherein the accumulation of MISC is about 25%.

63. The method of claim 61, wherein the accumulation of MISC is about 30%.

64. The method of claim 61, wherein the accumulation of MISC is about 40%.

65. The method of any one of claims 55-64, wherein the accumulation of MISC is measured as the OD₆₀₀ of the culture of the microorganism after bleach digest of the microorganism over the original OD₆₀₀ of the culture of the microorganism.

66. A biofertilizer for use to improve and/or maintain crop or plant yield, yield quality, or plant aesthetics, and/or improve soil health comprising a nitrogen-fixing microorganism, wherein the microorganism has an accumulation of a MISC greater than about 10%.

67. The biofertilizer of claim 66, wherein the biofertilizer is a liquid, a solid, or a semisolid, or a combination thereof.

68. The biofertilizer of claim 66 or 67, wherein the MISC is a PHA, a PolyP, or a lipid, or a combination thereof.

69. The biofertilizer of claim 68, wherein the MISC is a PHA.

70. The biofertilizer of claim 69, wherein the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof.

71. The biofertilizer of claim 70, wherein the PHA is PHB.

72. The biofertilizer of claim 66, wherein the accumulation of MISC is greater than about 10%, greater than about 12%, greater than about 15%, greater than about 20%, greater than about 25%, greater than about 30%, greater than about 35%, or greater than about 40%.

73. The biofertilizer of claim 72, wherein the accumulation of MISC is greater than about 25%.

74. The biofertilizer of claim 72, wherein the accumulation of MISC is greater than about 40%.

75. The biofertilizer of claim 71, wherein the accumulation of MISC is from about 10% to about 12%, from about 10% to about 15%, from about 10% to about 20%, from about 10% to about 25%, from about 12% to about 15%, from about 12% to about 20%, from about 12% to about 25%, from about 12% to about 30%, from about 15% to about 20%, from about 15% to about 25%, from about 15% to about 30%, from about 15% to about 35%, from about 20% to about 25%, from about 20% to about 30%, from about 20% to about 35%, from about 20% to about 40%, from about 25% to about 30%, from about 25% to about 35%, from about 25% to about 40%, from about 30% to about 40%, or from about 35% to about 40%.

76. The biofertilizer of claim 71, wherein the accumulation of MISC is about 10%, about 12%, about 15%, about 20%, about 25%, about 30%, about 35%, or about 40%.

77. The biofertilizer of claim 76, wherein the accumulation of MISC is about 25%.

78. The biofertilizer of claim 76, wherein the accumulation of MISC is about 30%.

79. The biofertilizer of claim 76, wherein the accumulation of MISC is about 40%.

80. The biofertilizer of any one of claims 70-79, wherein the accumulation of MISC is measured as the OD₆₀₀ of the culture of the microorganism after bleach digest of the microorganism over the OD₆₀₀ of the culture of the microorganism before bleach digest.

81. The biofertilizer of any one of claims 66-80, wherein the nitrogen-fixing microorganism comprises bacteria.

82. The biofertilizer of claim 81, wherein the nitrogen-fixing microorganism is a PHB-producing bacteria.

83. The biofertilizer of any one of claims 66-80, wherein the nitrogen-fixing microorganism comprises archea.

84. The biofertilizer of any one of claims 66-80, wherein the nitrogen-fixing microorganism comprises fungi.

85. The biofertilizer of any one of claims 81-84, wherein the nitrogen-fixing microorganism comprises one or more strains of Acidiphilium species, Acidiphilium multivorum, Alcaligenes species, Alcaligenes paradoxus, Arthrobacter species, Azohydromonas species, Azohydromonas australica, Azohydromonas species, Azohydromonas lata, Azospirillum species, Azospirillum amazonsense, Azospirillum lipoferum, Azospirillum thiophilum, Azotobacter vinelandii, Beggiatoa species, Beggiatoa alba, Beijerinckia species, Beijerinckia mobilis, Bradyrhizobium species, Bradyrhizobium elnakii, Bradyrhizobium japonicum, Bradyrhizobium japonicum (strain USDA 122), Burkholderia species, Burkholderia vietnameiensis, Cupriavidus species, Cupriavidus necator, Derxia species, Derxia gummosa, Herbaspirillum species, Herbaspirillum autrotrophicum, Hydrogenophaga species, Hydrogenophaga pseudoflava, Mesorhizobium species, Mesorhizobium alhagi, Methylibium species, Methylibium petroleiphilum, Methylocapsa species, Methylocapsa aurea, Methyloferula species, Methyloferula stellate, Methyloversatilis species, Methyloversatilis universalis, Microcyclus species, Microcyclus aquaticus, Microcyclus ebruneus, Nitrosococcus species, Nitrosococcus oceani, Nitrosomonas communis, Nitrospirillum amazonense, Nocardia species, Nocardia autotrophica, Nocardia opaca, Oligotropha species, Oligotropha carboxidovorans, Pannonibacter species, Pannonibacter phragmitetus, Paracoccus species, Paracoccus denitrificans, Paracoccus pantrophus, Paracoccus yeei, Pelagibaca species, Pelagibaca bermudensis, Pseudomonas species, Pseudomonas facilis, Pseudooceanicola species, Pseudooceanicola atlanticus, Ralstonia species, Ralstonia eutropha, Renobacter species, Renobacter vacuolatum, Rhizobium species, Rhizobium gallicum, Rhizobium japonicum, Rhodobacter species, Rhodobacter capsulatus, Rhodobacter sphaeroides, Rhodomicrobium species, Rhodomicrobium vannielii, Rubrivivax species, Rubrivivax gelatinosus, Salipiger species, Salipiger mucosus, Sinorhizobium species, Sinorhizobium americanum, Sinorhizobium fredii, Sinorhizobium meliloti, Skermanella species, Skermanella stibiiresistens, Stappia species, Stappia aggregate, Thauera species, Thauera humireducens, Variovorax species, Variovorax paradoxus, Xanthobacter species, and Xanthobacter autotrophicus, and any combinations thereof.

86. The biofertilizer of claim 85, wherein the nitrogen-fixing microorganism is Xanthobacter autotrophicus.

87. The biofertilizer of claim 85, wherein the nitrogen-fixing microorganism is Ralstonia eutropha.

88. The biofertilizer of any one of claims 66-87, wherein the crop or plant comprises one or more crops or plants from the following families: Asteraceae, Poaceae, Brassicaceae, Cucurbitaceae, Solanaceae, Rosaceae, Cannabaceae, Poaceae, Amaranthaceae, Amaryllidaceae, Polygonaceae, Liliaceae, Lamiaceae, Ericaceae or Fabaceae.

89. The biofertilizer of claim 88, wherein the crop or plant comprises one or more of the following crops or plants: a lettuce (Family: Asteraceae), a corn (Family: Poaceae), a cabbage (Family: Brassicaceae), a kale (Family: Brassicaceae), a cucumber (Family: Cucurbitaceae), a pepper (Family: Solanaceae), a tomato (Family: Solanaceae), a berry (Family: Rosaceae), a hemp (Family: Cannabaceae), a grass (Family: Poaceae), a turf, a beet (Family: Amaranthaceae), a squash (Family: Cucurbitaceae), an onion (Family: Amaryllidaceae), or a soybean (Family: Fabaceae).

90. The biofertilizer of claim 89, wherein the crop or plant comprises one or more of the following crops or plants: a baby leaf lettuce, a head lettuce, a sweet corn, a sweet pepper, a strawberry, or a raspberry.

91. The biofertilizer of any one of claims 66-90, wherein the biofertilizer is stable for at least 1 month, at least 2 months, at least 6 months, or at least 12 months at room temperature or at about 4° C.

92. The biofertilizer of claim 91, wherein the OD₆₀₀ of the biofertilizer changes within #10% for at least 1 month, at least 2 months, at least 6 months, or at least 12 months at room temperature or at about 4° C.

93. The biofertilizer of claim 91, wherein the amount of MISC changes within +10% for at least 1 month, at least 2 months, at least 6 months, or at least 12 months at room temperature or at about 4° C.

94. A method of improving and/or maintaining crop or plant yield, yield quality, or plant aesthetics and/or improving soil health comprising administered the biofertilizer of any one of claims 66-93 to the crop or plant.

95. The method of claim 94, wherein the crop or plant comprises one or more crops or plants from the following families: Asteraceae, Poaceae, Brassicaceae, Cucurbitaceae, Solanaceae, Rosaceae, Cannabaceae, Poaceae, Amaranthaceae, Amaryllidaceae, Polygonaceae, Liliaceae, Lamiaceae, Ericaceae or Fabaceae.

96. The method of claim 95, wherein the crop or plant comprises one or more of the following crops or plants: a lettuce (Family: Asteraceae), a corn (Family: Poaceae), a cabbage (Family: Brassicaceae), a kale (Family: Brassicaceae), a cucumber (Family: Cucurbitaceae), a pepper (Family: Solanaceae), a tomato (Family: Solanaceae), a berry (Family: Rosaceae), a hemp (Family: Cannabaceae), a grass (Family: Poaceae), a turf, a beet (Family: Amaranthaceae), a squash (Family: Cucurbitaceae), an onion (Family: Amaryllidaceae), or a soybean (Family: Fabaceae).

97. The method of claim 96, wherein the crop or plant comprises one or more of the following crops or plants: a baby leaf lettuce, a head lettuce, a sweet corn, a sweet pepper, a strawberry, or a raspberry.

98. A method of improving soil health comprising applying the biofertilizer of any one of claims 66-93 to a crop or plant in the soil.

99. The method of claim 98, wherein soil health is improved by reducing nitrogen leaching in the soil.

100. The method of claim 99, wherein the nitrogen leaching in the soil is reduced relative to soil that has not been administered the biofertilizer.

101. The method of claim 98, wherein soil health is improved by increasing biological nitrogen fixation in the soil.

102. The method of claim 101, wherein the biological nitrogen fixation is increased relative to soil that has not been administered the biofertilizer.

103. The medium of claim 32, wherein the nitrogen-fixing microorganism is Azotobacter vinelandii.

104. The biofertilizer of claim 85, wherein the nitrogen-fixing microorganism is Azotobacter vinelandii.
